(19) United States
(12) Patent Application Publication
(10) Pub. No.: US 2024/0230171 A1
(43) Pub. Date: Jul. 11, 2024

(54) METHODS, COMPOSITIONS AND SYSTEMS FOR SOLID-STATE BAROCALORIC APPLICATIONS

(71) Applicant: President and Fellows of Harvard College, Cambridge, MA (US)

(72) Inventors: Jarad A. MASON, Cambridge, MA (US); Jinyoung SEO, Cambridge, MA (US)

(21) Appl. No.: 18/285,899

(22) PCT Filed: Apr. 7, 2022
(86) PCT No.: PCT/US2022/023891

§ 371 Date: Oct. 6, 2023

Related U.S. Application Data
(60) Provisional application No. 63/171,938, filed on Apr. 7, 2021.

(51) Int. Cl. F25B 23/00; C07C 211/15; C07C 211/27; C07C 211/63; C07C 215/08; C07C 217/08; C07C 229/08

(52) U.S. Cl. CPC F25B 23/00; C07C 211/15; C07C 211/27; C07C 211/63; C07C 215/08; C07C 217/08; C07C 229/08

(57) ABSTRACT

The invention provides methods, compositions, and systems for barocaloric applications such as cooling, heating, and energy storage.

### BACKGROUND OF THE INVENTION

Currently, more than 20% of the world's electricity is used for cooling, including for the storage of foods and medicine, air conditioning of populated spaces and buildings, and more recently, removing heat from electronics and data centers. With the world's increasing population demanding more comfortable living conditions and smaller, faster, and more powerful electronics, the demand for cooling is expected to continue to increase. Conventional cooling technologies, which have been around since the early 19^{th} century, rely primarily on vapor compression cycles of hydrofluorocarbons that have a high global warming potential. In addition, next-generation electronics require aggressive cooling for sufficient thermal management, but current cooling methods cannot be scaled down to the dimensions of microchips. Thus, there is a pressing need to find new cooling technologies that are environmentally friendly and amenable to miniaturization.

Solid-state cooling based on caloric materials offers the potential to overcome many challenges associated with traditional cooling technologies. Caloric materials are a class of solids that undergo solid-solid phase transitions driven by magnetic, electrical, or mechanical stimuli. In a typical cooling cycle, the stimulus is applied adiabatically to induce a phase change—typically to a more ordered state—which leads to a large increase in temperature. Once the temperature has re-equilibrated by dissipating excess heat to a heat sink, the stimulus is removed, reverting the caloric material to its original phase—now at a lower temperature—that can be used to remove heat from a heat source. Because these phase transitions occur entirely in the solid state, refrigeration cycles can be achieved without using greenhouse gases. Until recently, materials exhibiting magnetocaloric, elastocaloric, and electrocaloric effects have been viewed as most promising. However, in magnetocaloric cooling, the requirement of a large magnetic field (H>2 T) and the cost of expensive rare-earth magnetic materials have prevented widespread industrial and commercial applications. In elastocaloric materials, a short fatigue life has limited their utilization. Electrocaloric materials have also lagged behind as they require the energetically expensive production of electric fields, whose value is limited by the breakdown field.

To date, many materials such as natural rubbers, shape-memory alloys and intermetallic compounds, antiferromagnetic compound (Mn₃GaN), ionic conductors (AgI), ferroelectric ceramic (BaTiO₃), ferrielectric organic salts, organic molecule-based switchable dielectrics, 3D hybrid perovskites (with a general chemical formula of ABX₃), and organic plastic crystals have been explored as barocaloric materials. However, these materials are often not ideal for practical refrigeration due to low thermodynamic efficiencies, small entropy changes associated with pressure-induced transitions (dozens of joules per kilogram per kelvin), small latent heats, the need to operate at non-ambient temperatures, large thermal hysteresis, high operating pressures, and a lack of mechanical durability and synthetic tunability. Most importantly, rational synthetic manipulation of these materials to establish fundamental structure-property relationships and to improve their performance is difficult.

Thus, there is a need for new methods, compositions, and systems for barocaloric applications.

### SUMMARY OF THE INVENTION

The invention provides methods, compositions, and systems for barocaloric applications, e.g., cooling, heating, and energy storage.

In one aspect, the invention provides a method of heating or cooling employing a barocaloric cycle including providing heat energy to a composition including an organic layer including optionally substituted C_{>3} alkyl chains (e.g., C_{>4} alkyl chains), wherein the organic layer is in a disordered state and wherein the organic layer is between first and second inorganic layers or includes a head group capable of hydrogen bonding, halogen bonding, and/or electrostatic interaction with a counterion (e.g., an anion such as a halide); applying compression to the composition to induce the organic layer to undergo an exothermic phase transition to an ordered state, releasing latent heat; removing the latent heat while the composition is compressed; and removing the compression to allow the composition to revert to the disordered state.

In some embodiments, the composition includes first and second inorganic layers separated by the organic layer. In some embodiments, the compression is hydrostatic or mechanical and/or the latent heat is removed by a heat sink. In some embodiments, the organic layer includes a C_{>3} alkyl ammonium species (e.g., C_{>4} alkyl, e.g., C_{4-36,} e.g., C₄₋₁₈), such as a species selected from:

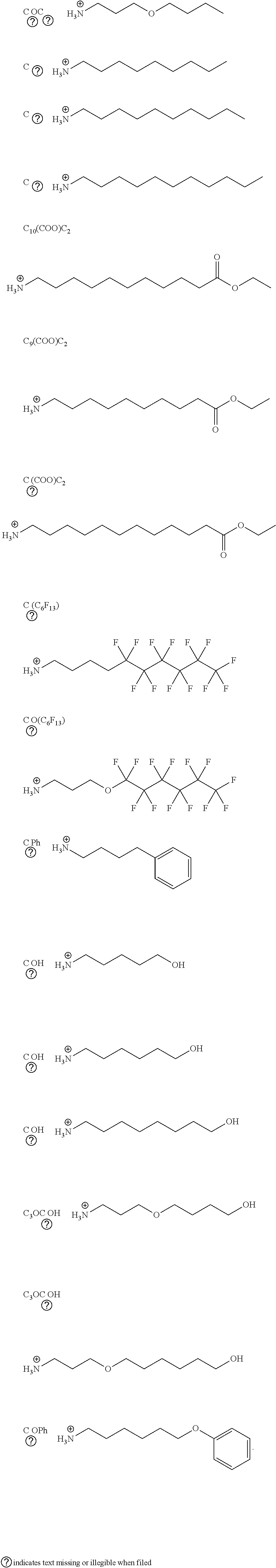

In some embodiments, the organic layer is an organic bilayer. In some embodiments, the organic layer includes a compound of formula (CₙH₂ₙ₊₁)(CₘH₂ₘ₊₁)NH₂X, where n is 1-3 or 4-36 and m is 4-36; and X is a monoanionic species (e.g., a halide (e.g., F, Cl, Br, or I) or a non-halide anion, such as NO₃—, ClO₃—, ClO₄—, H₂PO₄^{—}, HSO₄^{—}, CN^{—}, HCOO^{—}, N₃^{—}, N(CN)₂^{—}, BF₄^{—}, BH₄^{—}, PF₆^{—}, SCN^{—}, or OCN^{—}). In certain embodiments, n=m and n=4-36. In certain embodiments, n=1-3 and m=4-36 (e.g., where n=1 and m=6, 8, 10, 12, or 18).

In some embodiments, the composition is a 2D perovskite. In some embodiments, the 2D perovskite includes a transition metal halide. In some embodiments, the 2D perovskite includes Ti, V, Cr, Mn, Fe, Co, Ni, Cu, Zn, Nb, Mo, Rh, Pd, Cd, Re, Pt, or Hg. In some embodiments, the 2D perovskite includes a tetrahedral or octahedral transition metal complex. In some embodiments, the halide of the transition metal halide is F, Cl, Br, or I. In some embodiments, the transition metal halide includes a monovalent metal cation and a trivalent metal cation. In some embodiments, the 2D perovskite is of formula [(R¹)ₓ(R²)₁₋ₓ]₂MX_{y}X′_{4-y}, where R¹ and R² are independently optionally substituted alkylammonium species, X and X′ are different halides, x is a real number between 0-1, y is 0-4, M is a transition metal (e.g., Ti, V, Cr, Mn, Fe, Co, Ni, Cu, Zn, Nb, Mo, Rh, Pd, Cd, Re, Pt, or Hg); and where if y=0 or 4, R¹≠R² and x≠0 or 1.

In some embodiments, the organic layer includes two different molecular structures. In some embodiments, the first and second inorganic layers include a silicate. In some embodiments, the composition includes a metal alkyl phosphonate salt. In some embodiments, the composition includes a compound of the following table:

| Type | Chemical Formula |
|---|---|
| 2-D perovskite | (OA)₂MnCl₄ |
| | (OA)₂MnCl₄ |
| | (NA)₂MnCl₄ |
| | (NA)₂CuCl₄ |
| | (DA)₂CuCl₄ |
| Mixed 2D perovskites | (NA)₂CuCl₃Br |
| | (NA)₂CuCl₂Br₂ |
| | (NA)₂CuClBr₃ |
| | (DA)₂CuCl₃Br |
| | (DA)₂CuCl₂Br₂ |
| | (DA)₂CuClBr₃ |
| | [(NA)_{0.75}(DA)_{0.25}]₂CuCl₄ |
| | [(NA)_{0.5}(DA)_{0.5}]₂CuCl₄ |
| | [(NA)_{0.25}(DA)_{0.75}]₂CuCl₄ |
| | [(NA)_{0.25}(UA)_{0.75}]₂CuCl₄ |
| | [(NA)_{0.5}(UA)_{0.5}]₂CuCl₄ |
| | [(NA)_{0.5}(DA)_{0.5}]₂CuCl₂Br₂ |
| Di-n-alkyl ammonium salt | (n-C₆H₁₃)₂NH₂Br |
| | (n-C₈H₁₇)₂NH₂Cl |
| | (n-C₆H₁₃)₂NH₂Cl |
| | (n-C₆H₁₃)₂NH₂I |
| | (n-C₈H₁₇)₂NH₂Br |
| | (n-C₁₂H₂₅)₂NH₂Cl |
| | (n-C₁₂H₂₅)₂NH₂Br |
| | (n-C₁₀H₂₁)₂NH₂Cl |
| | (n-C₁₀H₂₁)₂NH₂Br |
| | (n-C₁₀H₂₁)₂NH₂I |
| | (n-C₁₂H₂₅)₂NH₂Br |
| | (n-C₁₈H₃₇)₂NH₂Cl |
| | (n-C₁₈H₃₇)₂NH₂Br |
| | (n-C₁₂H₂₅)(CH₃)NH₂Br |
| | (n-C₁₂H₂₅)(CH₃)NH₂Cl |
| Intercalation compound | FeOCl•C₁₄H₂₉NH₂ |
| (first-row transition metal) | Ni(CN)₂•C₁₂H₂₅NH₂ |
| | Ni(CN)₂• C₁₂H₂₅NH₂ |
| intercalated between | (C₁₈H₃₇)₃NH⁺ |
| montmorillonite (smectite) | Self-Assembled Monolayer |
| | (C₁₈H₃₇)₄N⁺ |
| | Self-Assembled Monolayer |
| Layered metallo- | Mg(O₃PC₂₂H₄₅) |
| alkylphosphonate | |

DA=decylammonium, NA=nonylammonium, and UA=undecylammonium.

In some embodiments, the compression results from a pressure change of less than 500 bar, e.g., less than 300 bar. In some embodiments, the compression results in a reversible entropy change of more than 200 J kg⁻¹ K⁻¹.

In some embodiments, the compression is provided using a pressure transmitting medium (PTM). In some embodiments, the method includes providing a gas to the PTM that induces a change in a thermal property of the composition. In some embodiments, the change in thermal property is a lowering of a phase transition temperature and/or a barocaloric effect inversion. In some embodiments, the method further includes removing the gas from the PTM. In some embodiments, the gas is an inert gas that permeates into a free volume of the organic layer. In some embodiments, permeated gas interacts with the composition. In some embodiments, permeation and interaction of the gas with the composition together induce a lowering of a phase transition and/or a barocaloric effect inversion. In some embodiments, the gas is nitrogen, argon, krypton, xenon, methane, ethane, propane, butane, sulfur hexafluoride, or carbon dioxide.

In an aspect, the invention provides a method of storing thermal energy employing by providing a composition including an organic layer including optionally substituted C_{>3} alkyl chains (e.g., C_{>4} alkyl chains, e.g., C₄₋₃₆ alkyl chains) at a first temperature and a first pressure, wherein the composition is in an ordered state and wherein the organic layer is between first and second inorganic layers or includes a head group capable of hydrogen bonding, halogen bonding, and/or electrostatic interaction with a counterion; and reducing compression on the composition to a second pressure to induce a phase transition in the composition to a disordered state, thereby storing energy.

In some embodiments, the method further includes increasing compression on the composition to apply a third pressure to revert the composition to an ordered state and release heat energy. In some embodiments, the composition includes first and second inorganic layers separated by the organic layer. In some embodiments, the compression is hydrostatic or mechanical. In some embodiments, the organic layer includes a C_{>3} alkyl (e.g., C_{>4} alkyl, e.g., C₄₋₃₆ alkyl) ammonium species, such as:

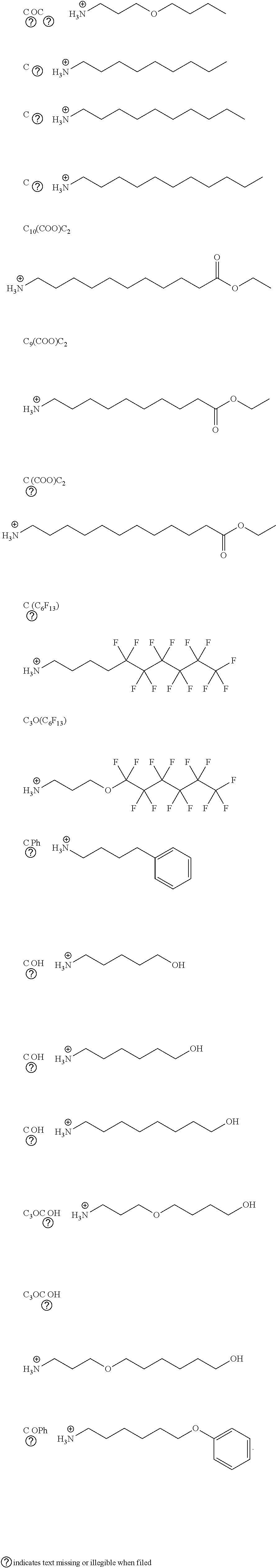

In some embodiments, the organic layer is an organic bilayer. In some embodiments, the organic layer includes a compound of formula (CₙH₂ₙ₊₁)(CₘH₂ₘ₊₁)NH₂X, where n is 1-3 or 4-36 and m is 4-36; and X is a monoanionic species (e.g., a halide (e.g., F, Cl, Br, or I) or a non-halide anion, such as NO₃^{—}, ClO₃^{—}, ClO₄^{—}, H₂PO₄^{—}, HSO₄^{—}, CN^{—}, HCOO^{—}, N₃^{—}, N(CN)₂^{—}, BF₄^{—}, BH₄^{—}, PF₆^{—}, SCN^{—}, or OCN^{—}). In certain embodiments, n=m and n=4-36. In certain embodiments, n=1-3 and m=4-36 (e.g., where n=1 and m=6, 8, 10, or 12).

In some embodiments, the composition is a 2D perovskite. In some embodiments, the 2D perovskite includes a transition metal halide. In some embodiments, the 2D perovskite includes Ti, V, Cr, Mn, Fe, Co, Ni, Cu, Zn, Nb, Mo, Rh, Pd, Cd, Re, Pt, or Hg. In some embodiments, the 2D perovskite includes a tetrahedral or octahedral transition metal complex. In some embodiments, the halide of the transition metal halide is F, Cl, Br, or I. In some embodiments, the transition metal halide includes a monovalent metal cation and a trivalent metal cation. In some embodiments, the 2D perovskite is of formula [(R¹)ₓ(R²)₁₋ₓ]₂MX_{y}X′_{4-y}, where R¹ and R² are independently optionally substituted alkylammonium species, X and X′ are different halides, x is a real number between 0-1, y is 0-4, M is a transition metal (e.g., Ti, V, Cr, Mn, Fe, Co, Ni, Cu, Zn, Nb, Mo, Rh, Pd, Cd, Re, Pt, or Hg); and where if y=0 or 4, R¹≠R² and x≠0 or 1.

In some embodiments, the organic layer includes two different molecular structures. In some embodiments, the inorganic layer includes a silicate. In some embodiments, the composition includes a metal alkyl phosphonate salt.

In some embodiments, the composition includes a compound of the following table:

| Type | Chemical Formula |
|---|---|
| 2-D perovskite | (OA)₂MnCl₄ |
| | (OA)₂MnCl₄ |
| | (NA)₂MnCl₄ |
| | (NA)₂CuCl₄ |
| | (DA)₂CuCl₄ |
| Mixed 2D perovskites | (NA)₂CuCl₃Br |
| | (NA)₂CuCl₂Br₂ |
| | (NA)₂CuClBr₃ |
| | (DA)₂CuCl₃Br |
| | (DA)₂CuCl₂Br₂ |
| | (DA)₂CuClBr₃ |
| | [(NA)_{0.75}(DA)_{0.25}]₂CuCl₄ |
| | [(NA)_{0.5}(DA)_{0.5}]₂CuCl₄ |
| | [(NA)_{0.25}(DA)_{0.75}]₂CuCl₄ |
| | [(NA)_{0.25}(UA)_{0.75}]₂CuCl₄ |
| | [(NA)_{0.5}(UA)_{0.5}]₂CuCl₄ |
| | [(NA)_{0.5}(DA)_{0.5}]₂CuCl₂Br₂ |
| Di-n-alkyl ammonium salt | (n-C₆H₁₃)₂NH₂Br |
| | (n-C₈H₁₇)₂NH₂Cl |
| | (n-C₆H₁₃)₂NH₂Cl |
| | (n-C₆H₁₃)₂NH₂I |
| | (n-C₈H₁₇)₂NH₂Br |
| | (n-C₁₂H₂₅)₂NH₂Cl |
| | (n-C₈H₁₇)₂NH₂I |
| | (n-C₁₀H₂₁)₂NH₂Cl |
| | (n-C₁₀H₂₁)₂NH₂Br |
| | (n-C₁₀H₂₁)₂NH₂I |
| | (n-C₁₂H₂₅)₂NH₂Br |
| | (n-C₁₈H₃₇)₂NH₂Cl |
| | (n-C₁₈H₃₇)₂NH₂Br |
| | (n-C₁₂H₂₅)(CH₃)NH₂Br |
| | (n-C₁₂H₂₅)(CH₃)NH₂Cl |
| Intercalation compound | FeOCl•C₁₄H₂₉NH₂^{g} |
| (first-row transition metal) | Ni(CN)₂•C₁₂H₂₅NH₂^{h} |
| | Ni(CN)₂•C₁₂H₂₅NH₂^{h} |
| intercalated between | (C₁₈H₃₇)₃NH⁺ |
| montmorillonite (smectite) | Self-Assembled Monolayer |
| | (C₁₈H₃₇)₄N⁺ |
| | Self-Assembled Monolayer |
| Layered metallo- | Mg(O₃PC₂₂H₄₅) |
| alkylphosphonate | |

In some embodiments, the compression is provided using a pressure transmitting medium (PTM) and the method includes providing a gas to the PTM that induces a change in a thermal property of the composition. In some embodiments, the gas is an inert gas that is able to permeate a free volume of the organic layer. In some embodiments, the permeated gas interacts with the composition. In some embodiments, permeation and interaction of the gas with the composition together induce a lowering of a phase transition and/or a barocaloric effect inversion. In some embodiments, the gas is nitrogen, argon, krypton, xenon, methane, ethane, propane, butane, sulfur hexafluoride, or carbon dioxide.

In some embodiments, the change in thermal property is a lowering of a phase transition temperature and/or a barocaloric effect inversion.

In an aspect, the invention provides a 2D perovskite composition including first and second layers of a transition metal halide and an organic layer including a C_{>3} alkyl (e.g., C_{>4} alkyl, e.g., C₄₋₃₆ alkyl chains) ammonium species selected from:

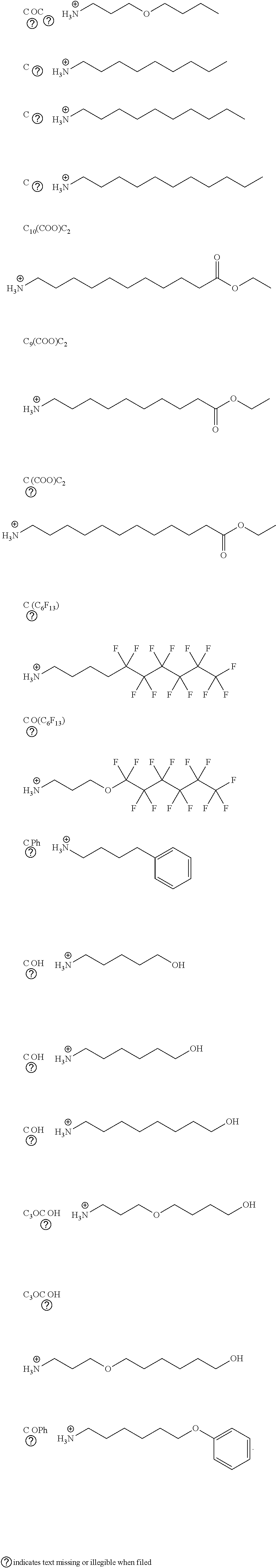

In an aspect, the invention provides a 2D perovskite composition having formula [(R¹)ₓ(R²)₁₋ₓ]₂MX_{y}X′_{4-y}, where R¹ and R² are independently optionally substituted alkylammonium species, X and X′ are different halides, x is between 0-1, y is 0-4, M is a transition metal (e.g., Ti, V, Cr, Mn, Fe, Co, Ni, Cu, Zn, Nb, Mo, Rh, Pd, Cd, Re, Pt, or Hg); and where if y=0 or 4, R¹≠R² and x≠0 or 1.

In some embodiments, R¹ and R² are independently alkylammonium species of formula C₂H₂ₙ₊₁NH₃⁺, where n>3 (e.g., n>4, e.g., C₄₋₃₆ alkyl). In some embodiments, the 2D perovskite composition has a formula selected from: (NA)₂CuCl₃Br; (NA)₂CuCl₂Br₂; (NA)₂CuClBr₃; (DA)₂CuCl₃Br; (DA)₂CuCl₂Br₂; (DA)₂CuClBr₃; [(NA)_{0.75}(DA)_{0.25}]₂CuCl₄; [(NA)_{0.5}(DA)_{0.5}]₂CuCl₄; [(NA)_{0.25}(DA)_{0.75}]₂CuCl₄; [(NA)_{0.25}(UA)_{0.75}]₂CuCl₄; [(NA)_{0.5}(UA)_{0.5}]₂CuCl₄; or [(NA)_{0.5}(DA)_{0.5}]₂CuCl₂Br₂. In some embodiments, R¹ and R² are independently alkylammonium species selected from:

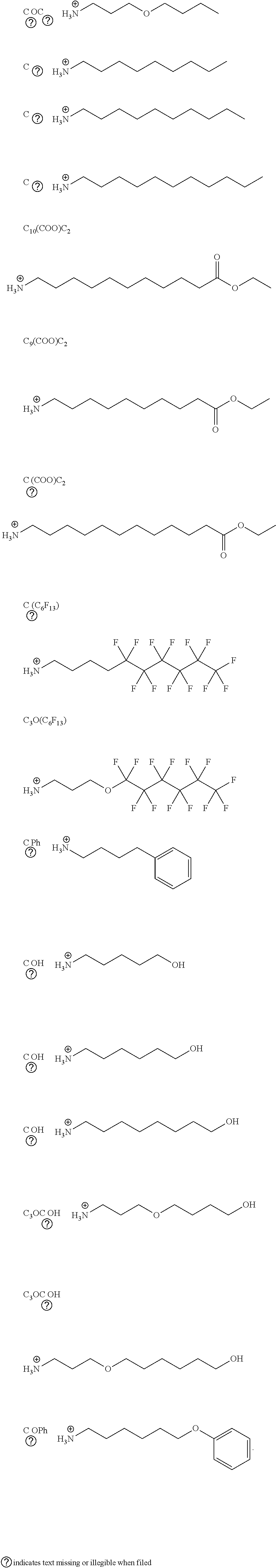

In some embodiments, X is Cl, and X′ is Br.

In an aspect, the invention provides a barocaloric system including a composition including an organic layer including optionally substituted C_{>3} alkyl chains (e.g., C_{>4} alkyl chains, e.g., C₄₋₃₆ alkyl chains), wherein the organic layer is between first and second inorganic layers or includes a head group capable of hydrogen bonding, halogen bonding, and/or electrostatic interaction with a counterion; and a source of compression.

In some embodiments, the system includes first and second inorganic layers separated by the organic bilayer. In some embodiments, the organic layer includes a compound of formula (CₙH₂ₙ₊₁)(CₘH₂ₘ₊₁)NH₂X, where n is 1-3 or 4-36 and m is 4-36; and where X is a monoanionic species (e.g., a halide (e.g., F, Cl, Br, or I) or a non-halide anion, such as NO₃—, ClO₃—, ClO₄—, H₂PO₄—, HSO₄—, CN^{—}, HCOO^{—}, N₃^{—}, N(CN)₂^{—}, BF₄^{—}, BH₄^{—}, PF₆^{—}, SCN^{—}, or OCN^{—}). In some embodiments, n=m and n=4-36. In some embodiments, n=1-3 and m=4-36 (e.g., where n=1 and m=6, 8, 10, or 12).

In some embodiments, the source of compression is hydrostatic or mechanical. In some embodiments, the system further includes a heat sink.

In an aspect, the invention provides a barocaloric system. The system includes a composition including an organic layer including optionally substituted C_{>3} alkyl chains. The system further includes a pressure transmitting medium including one or more gases, at least one of which induces a change in a thermal property of the organic layer, and a source of compression.

In some embodiments, the at least one gas is an inert gas that is able to permeate a free volume of the organic layer. In some embodiments, the permeated gas interacts with the composition. In some embodiments, an extent of permeation and interaction of the at least one gas with the composition together induce a lowering of a phase transition and/or a barocaloric effect inversion. In some embodiments, the change in thermal property is a lowering of a phase transition temperature and/or a barocaloric effect inversion. In some embodiments, the system includes a pump for controlling the amount of the at least one gas. In some embodiments, the system includes a heat sink. In some embodiments, the system further includes a second organic layer that does not undergo the barocaloric effect inversion. In some embodiments, the at least one gas is nitrogen, argon, krypton, xenon, methane, ethane, propane, butane, sulfur hexafluoride, or carbon dioxide.

### Definitions

The term “about,” as used herein, refers to ±10% of a recited value.

The term “alkyl,” as used herein, is meant straight chain or branched saturated groups of carbons. Alkyl groups are exemplified by n-, sec-, iso- and tert-butyl, neopentyl, nonyl, decyl, and the like, and may be optionally substituted with one or more, substituents. Alkyl groups of the invention may include 1 or more carbon atoms, e.g., greater than 2, e.g., 6-15, such as 8-12, in the main chain. Carbon atoms in the main chain may be interrupted with one or more heteroatoms, e.g., O, S, or N.

By “aryl” is meant an aromatic cyclic group in which the ring atoms are all carbon. Exemplary aryl groups include phenyl, naphthyl, and anthracenyl. Aryl groups may be optionally substituted with one or more substituents.

By “carbocyclyl” is meant a non-aromatic cyclic group in which the ring atoms are all carbon. Exemplary carbocyclyl groups include cyclopropyl, cyclobutyl, cyclopentyl, cyclohexyl, cycloheptyl, and cyclooctyl. Carbocyclyl groups may be optionally substituted with one or more substituents.

By “halo” is meant, fluoro, chloro, bromo, or iodo.

By “heteroaryl” is meant an aromatic cyclic group in which the ring atoms include at least one carbon and at least one O, N, or S atom, provided that at least three ring atoms are present. Exemplary heteroaryl groups include oxazolyl, isoxazolyl, tetrazolyl, pyridyl, thienyl, furyl, pyrrolyl, imidazolyl, pyrimidinyl, thiazolyl, indolyl, quinolinyl, isoquinolinyl, benzofuryl, benzothienyl, pyrazolyl, pyrazinyl, pyridazinyl, isothiazolyl, benzimidazolyl, benzothiazolyl, benzoxazolyl, oxadiazolyl, thiadiazolyl, and triazolyl. Heteroaryl groups may be optionally substituted with one or more substituents.

By “heterocyclyl” is meant a non-aromatic cyclic group in which the ring atoms include at least one carbon and at least one O, N, or S atom, provided that at least three ring atoms are present. Exemplary heterocyclyl groups include epoxide, thiiranyl, aziridinyl, azetidinyl, thietanyl, dioxetanyl, morpholinyl, thiomorpholinyl, piperazinyl, piperidinyl, pyrrolidinyl, tetrahydropyranyl, tetrahydrofuranyl, dihydrofuranyl, tetrahydrothienyl, dihydrothienyl, dihydroindolyl, tetrahydroquinolyl, tetrahydroisoquinolyl, pyranyl, pyrazolinyl, pyrazolidinyl, dihydropyranyl, tetrahydroquinolyl, imidazolinyl, imidazolidinyl, pyrrolinyl, oxazolidinyl, isoxazolidinyl, thiazolidinyl, isothiazolidinyl, dithiazolyl, and 1,3-dioxanyl. Heterocyclyl groups may be optionally substituted with one or more substituents.

Optional substituents include halo, optionally substituted C₃₋₁₀ carbocyclyl; optionally substituted C₁₋₉ heterocyclyl having one to four heteroatoms independently selected from O, N, and S; optionally substituted C₆₋₂₀ aryl; optionally substituted C₁₋₉ heteroaryl having one to four heteroatoms independently selected from O, N, and S; —CN: —NO₂; —ORₐ; —N(Rₐ)₂; —C(═O)Rₐ; —C(═O)ORₐ; —S(═O)₂Rₐ; —S(═O)₂ORₐ; —P(═O)Rₐ₂; —O—P(═O)(ORₐ)₂, or —P(═O)(ORₐ)₂, or an ion thereof; wherein each Rₐ is independently H, optionally substituted C₁₋₆ alkyl; optionally substituted C₃₋₁₀ carbocyclyl; optionally substituted C₁₋₉ heterocyclyl having one to four heteroatoms independently selected from O, N, and S; optionally substituted C₆₋₂₀ aryl; or optionally substituted C₁₋₉ heteroaryl having one to four heteroatoms independently selected from O, N, and S.

### BRIEF DESCRIPTION OF THE DRAWINGS

FIGS. 1A-1B show powder X-ray diffraction data for (C₁₀H₂₁NH₃)₂MnCl₄ under variable pressures of He obtained at 17-BM-B. Powder patterns at 31° C. (FIG. 1A) show exclusively an ordered phase. Powder patterns obtained at 42° C. (FIG. 1B) show an expanded, disordered phase. Between 37-41° C., the sample undergoes order-to-disorder transition, exhibiting an increase in the inter-layer spacing. The simulated powder pattern at the bottom is from room temperature crystal structure of the same compound in ordered phase and used as a reference. The peaks from (001) reflections shifted to lower angles, indicating the increase in inter-layer distance. The inter-layer calculated based on the peak positions is 28.74 Å (˜7.3% increase).

FIG. 2 shows variable-temperature powder X-ray diffraction data (C₁₀H₂₁NH₃)₂MnCl₄ at 1 bar. The thermally-induced order-disorder phase transition shifts to a much higher temperature at higher pressure, demonstrating the pressure-dependence of this phase transition.

FIG. 3 shows structural and thermal properties of two-dimensional layered perovskites of the invention. Four different types of functionalized long-chain organic molecules were incorporated to yield (C₁₀H₂₁NH₃)₂[MnCl₄] (denoted as (C₁₀)₂[MnCl₄]), (HO—C₈H₁₇NH₃)₂[CuCl₄] (denoted as (C₈OH)₂[CuCl₄]), (C₄H₉OC₃H₆NH₃)₂[CuCl₄] (denoted as (C₃OC₄)₂[CuCl₄]), and (C₆H₅C₄H₈NH₃)₂[CuCl₄] (denoted as (C₄Ar)₂[CuCl₄]). Left: Powder X-ray diffraction patterns of (C₁₀)₂[MnCl₄], (C₈OH)₂[CuCl₄], (C₃OC₄)₂[CuCl₄], and (C₄Ar)₂[CuCl₄]. These patterns were compared to the simulated pattern of (C₁₀)₂[MnCl₄]. In the powder patterns, (001) reflections were primarily observed due to the strong preferred orientation of the samples. The inter-layer spacing for each compound can be calculated based on the peak positions. The powder patterns reveal the high crystallinity and phase purity of all samples. Right: Differential scanning calorimetry (DSC) traces of (C₁₀)₂[MnCl₄], (C₈OH)₂[CuCl₄], (C₃OC₄)₂[CuCl₄], and (C₄Ar)₂[CuCl₄]. Inset: transition temperatures Tₜᵣ, transition enthalpy ΔH, and transition enthalpy ΔS. The transition entropy was calculated by: ΔH=TₜᵣΔS. DSC measurements reveal that the samples undergo thermally-induced, reversible solid-solid phase transitions involving large changes in entropy. The gravimetry entropy changes of the samples are higher than 100 J kg⁻¹ K⁻¹. Notably, the transition enthalpy of (C₁₀)₂[MnCl₄] is ˜221 J kg⁻¹ K⁻¹. In terms of the entropy change, this material is expected to be competitive to some of the best current caloric materials.

FIGS. 4A-4B show barocaloric cooling with two-dimensional (2-D) metal-halide perovskite. FIG. 4A shows an illustration of a barocaloric cooling cycle driven by pressure-induced order-disorder transitions in a 2-D perovskite. The large barocaloric effects in the 2-D perovskite arise from a chain-melting phase transition of the hydrocarbon chains associated with large changes in volume and conformational entropy. For a conventional barocaloric effect, a cooling cycle begins with an adiabatic (Brayton-like cycle) or isothermal (Stirling-like cycle) increase in pressure that induces a transition from an expanded, high-entropy phase of a material to a contracted, low-entropy phase. Heat released during this exothermic transition is dissipated to a heat sink, returning the material to its original temperature but now at a lower entropy. The pressure is then adiabatically or isothermally decreased to reverse the phase transition, which leads to cooling of a heat source. FIG. 4B is a comparison of entropies of transition, ΔSₜᵣ, and transition temperature, Tₜᵣ, for select 2-D perovskites with long hydrocarbon chains, (CₙH₂ₙ₊₁NH₃)₂MX₄ (n=7-16; M=Mn, Cd, Cu, Pb; X=Cl, Br, I). The thermally induced phase transitions in 2-D perovskites are accompanied with large changes in entropies and sensitive to the length of hydrocarbon chain and identity of metal-halide layer. Comprehensive summary of phase-change properties is provided in Tables 2 to 4 (ΔSₜᵣ and Tₜᵣ). In addition, the dependence of phase transition to hydrostatic pressure, dTₜᵣ/dP, is estimated for select 2-D perovskites in Table 5.

FIGS. 5A-5H show thermally induced chain-melting transitions in (DA)₂MnCl₄ and (NA)₂CuBr₄ at ambient pressure. FIGS. 5A and 5B show differential scanning calorimetry (DSC) traces for (DA)₂MnCl₄ (FIG. 5A) and (NA)₂CuBr₄ (FIG. 5B) with heating and cooling rates of 2 K min⁻¹. ΔSₜᵣ and enthalpies of transition (ΔHₜᵣ) are shown with thermal hysteresis (ΔT_{hys}) highlighted in grey area. Note that ΔT_{hys} is calculated as the difference between heating and cooling transition onset temperatures, with ΔT_{hys}=T_{tr, heating}-T_{tr, cooling}. FIGS. 5C and 5D show the temperature dependence of specific volumes for (DA)₂MnCl₄ (FIG. 5C) and (NA)₂CuBr₄ (FIG. 5D) obtained from variable-temperature powder X-ray diffraction and He pycnometry measurements, revealing large volume changes for the transitions, ΔV_{PXRD} and ΔV_{pyc}, respectively. FIGS. 5E and 5G show conformations of alkylammonium (CₙH₂ₙ₊₁NH₃₊) chains in the low-temperature (LT) phase crystal structures of (DA)₂MnCl₄ (n=10) (FIG. 5E) and (NA)₂CuBr₄ (n=9) (FIG. 5G) at 270 K. Atomic displacement parameters are shown at 50% probability for CₙH₂ₙ₊₁NH₃⁺ chains. Note that C₉H₁₉NH₃⁺ chains are disordered over two positions. FIGS. 5F and 5H show variable-temperature crystal structures of LT and high-temperature (HT) phases of (DA)₂MnCl₄ (FIG. 5F) and (NA)₂CuBr₄ (FIG. 5H) that feature order-disorder chain-melting transitions in the organic bilayers. Note that the HT phase crystal structures were obtained at 330 K and 335 K for (DA)₂MnCl₄ and (NA)₂CuBr₄, respectively. Purple, orange, green, brown, grey, and blue spheres represent Mn, Cu, Cl, Br, C, and N atoms, respectively. H atoms are omitted for clarity.

FIGS. 6A-6J show barocaloric effects in 2-D metal-halide perovskites. FIGS. 6A and 6D show DSC measurements at applied pressures for (FIG. 6A) (DA)₂MnCl₄ and (FIG. 6D) (NA)₂CuBr₄ with heating and cooling rates of 2 K min⁻¹. Applications of hydrostatic He pressure increases Tₜᵣ. FIGS. 6B and 6E show isothermal entropy change, ΔSᵢₜ, calculated by the quasi-direct method for (DA)₂MnCl₄ (FIG. 6B) and (NA)₂CuBr₄ (FIG. 6E) on heating and cooling for operating pressures from 40 bar to 150 bar, with shaded area indicating the reversible ΔSᵢₜ. The reversible values of ΔSᵢₜ can be estimated from the overlap between compression-induced and decompression-induced ΔSᵢₜ curves reflected across the temperature axis. FIGS. 6C and 6F show direct evaluation of pressure hysteresis, ΔP_{hys}, through quasi-isothermal pressure cycling DSC measurements for (DA)₂MnCl₄ (FIG. 6C) and (NA)₂CuBr₄, (FIG. 6F) at 311 K and 307 K, respectively, between 1 and 150 bar pressures. ΔP_{hys} is calculated as the difference between the onset pressures for compression-induced exotherms and decompression-induced endotherms, indicated by the horizontal dashed green lines. From the pressure dependence of heating and cooling onset temperatures, ΔP_{hys} are predicted to be 73 bar and 16 bar for (DA)₂MnCl₄ and (NA)₂CuBr₄, at 311 K and 307 K, respectively. FIGS. 6G and 6I show variable-temperature powder X-ray diffraction (PXRD) patterns for (DA)₂MnCl₄ (FIG. 6G) and (NA)₂CuBr₄ (FIG. 6I) at 360 bar and 300 bar He pressures, respectively, during cooling, with X-ray wavelength of 0.45237 Å. FIGS. 6H and 6J show the pressure dependence of transition temperature, barocaloric coefficient dTₜᵣ/dP, for (DA)₂MnCl₄ (FIG. 6H) and (NA)₂CuBr₄ (FIG. 6J), measured through HP-DSC and in situ PXRD experiments. Red and blue symbols indicate T_{tr, heating} and T_{tr, cooling,} respectively.

FIGS. 7A-7B show properties of representative barocaloric materials. Comparison of (FIG. 7A) ΔSₜᵣ and Tₜᵣ, and (FIG. 7B) ΔT_{hys}, barocaloric coefficient dTₜᵣ/dP, and Pᵣₑᵥ for leading barocaloric materials. ΔSₜᵣ and Tₜᵣ determined for endothermic transitions are shown. Pᵣₑᵥ, calculated through Pᵣₑᵥ=ΔT_{hys}/|dTₜᵣ/dP|, corresponds to the slope of the plot, and dTₜᵣ/dP values for exothermic and endothermic transitions are used for conventional and inverse barocaloric materials, respectively. For comparison purpose, ΔSₜᵣ is highlighted as an estimate for barocaloric effect ΔSᵢₜ, since ΔSₜᵣ at ambient pressure represent the maximum entropy change for a pressure-induced phase transition. Note that reported ΔSᵢₜ values are heavily influenced by measurement conditions, such as operating pressure. Comprehensive evaluation of barocaloric properties, including reversible and irreversible ΔSᵢₜ values, is provided in Tables 18 and 19. Additionally, barocaloric effects associated with chain-melting transitions in other types of compounds are also estimated in Table 20.

FIGS. 8A-8C show a pressure-tunable thermal energy storage using chain-melting phase transition. Phase transition temperature Tₜᵣ during thermal energy storage can be tuned by application and removal of hydrostatic pressure. FIG. 8A shows a typical PT-TES cycle and for materials with conventional barocaloric effects (dTₜᵣ/dP>0), the material is first charged with thermal energy at the storage temperature Tₛₜₒᵣ and initial pressure P, through disordering transition (ΔSₜᵣ>0), hydrostatic pressure of ΔP is then applied to the material outside of the transition. Under the compressed environment, the temperature at which the thermal energy is released (release temperature, Tᵣₑₗ) shift to a higher temperature, by (dTₜᵣ/dP)×ΔP. After the stored heat is released, the pressure returns to the original starting pressure P. Note that this cycle can be readily reversed, such that the thermal energy is stored at a higher temperature through the application of pressure ΔP and released at a lower temperature when the pressure is removed. In FIGS. 8B and 8C the temperature span is calculated through the equation ΔTₛₚₐₙ=(dTₜᵣ/dP)×ΔP−ΔT_{hys}, and materials with high barocaloric coefficients and low hysteresis can lead to large temperature span under small operating pressure. With the operating pressure ΔP, as long as the temperature lies within the temperature span, application or release of the pressure can be exploited to realize on-demand thermal energy storage. In this approach, the thermal energy can be released and stored by applying and removing pressure, respectively. In addition to (DA)₂MnCl₄ and (NA)₂CuBr₄ highlighted in this manuscript, other types of compounds containing long-chain hydrocarbons (Table 20), including other 2-D perovskites with different chain length and metal-halide layers (Tables 1 to 5), are predicted to be competitive materials for PT-TES.

FIG. 9 illustrates on-demand tuning of phase-change temperatures through pressure. Application of pressure can be used to adjust the phase-change temperature of the TES material on demand, such that the working temperature can be optimized for changing demands of a thermal energy storage. This approach can dramatically increase the versatility of a TES material, as it can store and release the thermal energy across a broad temperature range. Note that the concept of PT-TES can be applied to thermal managements, where an abrupt increase in thermal load at a high temperature (T_{high}) can be reduced by shifting the storage temperature of a TES material such that it matches with T_{high}. In this condition, the heat can be removed more efficiently and rapidly.

FIGS. 10A-10D show variable-temperature infrared spectra for (DA)₂MnCl₄ and (NA)₂CuBr₄. In FIG. 10A the LT and HT phase spectra were collected 5 K below and 5 K above the phase transition temperature, respectively. FIGS. 10B-10D show zoomed in views of three different regions with bands that correspond to NH₃ bending (FIG. 10B) and CH₂ bending, CH₃ bending and CH₂ wagging (FIG. 10C), and CH₂ rocking bands (FIG. 10D). Key shifts in peak positions are indicated by vertical grey bars, and CH₂ wagging bands associated with conformational defects are highlighted with red and purple dashed lines for (DA)₂MnCl4 and (NA)₂CuBr₄, respectively. Note that previous reports on (CₙH₂ₙ₊₁NH₃)₂MCl₄ (M=Cd, Mn) perovskites revealed that the peak near 1337 cm⁻¹ does not depend on chain conformation. The IR bands used for conformational analysis are summarized in Table 6.

FIG. 11 shows images of mixed halide 2-D perovskites. With increasing bromide concentration, the color of the crystalline compounds changes noticeably, from a bright yellow to an orange, deep red, and finally dark purple.

FIGS. 12A and 12B show thermodynamic properties of newly discovered barocaloric materials. FIG. 12A shows a comparison of entropy change, ΔSₜᵣ, and transition temperature, Tₜᵣ, for the mixed halide and mixed cation 2-D perovskites contrasted with non-mixed perovskites (NA)₂CuBr₄ and (DA)₂MnCl_{4.} FIG. 12B shows comparisons of thermal hysteresis, ΔT_{hys}, and barocaloric coefficient, dT/dP, for the five mixed 2-D perovskites as well as (DA)₂MnCl₄ and (NA)₂CuBr₄. Note that the minimum operating pressure values (Pᵣₑᵥ) were calculated based on peak transition temperatures due the broadness of the DSC peaks, and thus Pᵣₑᵥ shown here are approximately three times higher than the values calculated based on onset ΔThys values. All five materials fall within an easily accessible pressure range (71-108 bar), in comparison to previously reported barocaloric compounds (400-4000 bar). As indicated by grey dashed lines, Pᵣₑᵥ values for [(NA)_{0.5}(DA)_{0.5}]2CuCl₄ and [(NA)_{0.5}(DA)_{0.5}]₂CuCl₂Br₂ are 71 bar and 108 bar, respectively, both of which are lower than Pᵣₑᵥ of (DA)₂MnCl₄ (dashed green line).

FIGS. 13A-13C Variable-temperature powder X-ray diffraction (PXRD) patterns for (DA)₂CuCl₂Br₂ at 1 bar of He obtained while cooling from 331 K to 289 K, with an X-ray wavelength of 0.45213 Å. In FIGS. 13A and 13B the PXRD patterns are shown at variable temperatures (as indicated), with grey shades highlighting (001), (002), and (003) reflections. The red and blue patterns correspond to the high-temperature (HT) and low-temperature (LT) phases, respectively, with purple indicating patterns in which both phases are present during the transition from HT to LT phase. FIG. 13C shows a waterfall plot for the variable-temperature PXRD data, with a dashed line indicating the transition temperature (Tₜᵣ). Note that the sample was cooled with a cooling rate of 3 K min⁻¹.

FIGS. 14A-14C show waterfall plots for variable-temperature powder X-ray diffraction (PXRD) data for (DA)₂CuCl₂Br₂ at (FIG. 14A) 80 bar and (FIG. 14B) 300 bar of He obtained while cooling from 335 K to 274 K, with an X-ray wavelength of 0.45213 Å. Dashed lines indicate the transition temperatures (Tₜᵣ). Note that the sample was cooled with a cooling rate of 3 K min⁻¹. FIG. 14C shows the pressure dependence of the chain-melting transition temperature as determined by HP-DSC (squares) and PXRD (diamonds) is used to calculate the barocaloric coefficient (dT/dP) for (DA)₂CuCl₂Br₂.

FIG. 15 shows the temperature dependence of interlayer distances for (DA)₂CuCl₂Br₂ obtained from isobaric PXRD experiments during cooling.

FIGS. 16A-16D show powder X-ray diffraction (PXRD) patterns for mixed-halide (NA)₂CuCl₄₋ₓBrₓ perovskites at 1 bar of He, with an X-ray wavelength of 0.45213 Å, for (FIGS. 16A-16B) high-temperature (HT) and (FIGS. 16C-16D) low-temperature (LT) phase at 311 K and 269 K, respectively. The PXRD patterns are shown at variable halide compositions (as indicated), with grey shades highlighting (002) and (003) reflections that are used to calculate the interlayer distance. Peak positions of (002) and (003) reflections for (NA)₂CuCl₄ and (NA)₂CuBr₄ are indicated using dashed lines. The compounds contain nonylammonium (NA, C₉) chains confined within the Cu—X pocket (X=Cl, Br).

FIGS. 17A-17B show relationships between interlayer distance and other properties of the materials. FIG. 17A shows interlayer distances for mixed-halide (NA)₂CuCl₄₋ₓBrₓ perovskites for high-temperature (HT) and low-temperature (LT) phase at 311 K and 269 K, respectively. The change in interlayer distance (Δd), indicated by a dashed arrow, is calculated as the difference between the interlayer distances for HT phase (dHT) and LT phase (dLT), with Δd=dHT−dLT. FIG. 17B shows relationships between the relative change in interlayer distance (Δd/dLT) and the transition entropy (ΔStr) associated with chain-melting transitions.

FIG. 18 shows relationships between the relative change in interlayer distance (Δd/d_{LT}) and the transition entropy (ΔSₜᵣ) associated with chain-melting transitions for mixed-halide (NA)₂CuCl₄₋ₓBrₓ perovskites. Mixed halide perovskite with DA chain (n=10): volume change (ΔV) and gravimetric entropy change (ΔS).

FIGS. 19A-19D show powder X-ray diffraction (PXRD) patterns for mixed-chain [(NA)₁₋ₓ(DA)ₓ]₂CuCl₄ perovskites at 1 bar of He, with an X-ray wavelength of 0.45213 Å, for (FIGS. 19A-19B) high-temperature (HT) and (FIGS. 19C-19D) low-temperature (LT) phase at 321 K and 279 K, respectively. The PXRD patterns are shown at variable chain compositions (as indicated), with grey shades highlighting (002) and (003) reflections that are used to calculate the interlayer distance. Peak positions of (002) and (003) reflections for (NA)₂CuCl₄ and (DA)₂CuCl₄ are indicated using dashed lines. The compounds contain nonylammonium (NA, C₉) and decylammonium (DA, C₁₀) chains confined within the Cu—Cl pocket.

FIGS. 20A and 20B Mixed chain perovskites: volume change (ΔV) and molar entropy change (ΔS). FIG. 20A shows interlayer distances for mixed-chain [(NA)₁₋ₓ(DA)ₓ]₂CuCl₄ perovskites for high-temperature (HT) and low-temperature (LT) phase at 321 K and 279 K. respectively. Red and blue symbols indicate the interlayer distances in HT and LT phase, respectively. The change in interlayer distance (Δd), indicated by a dashed arrow, is calculated as the difference between the interlayer distances for HT phase (d_{HT}) and LT phase (d_{LT}), with Δd=d_{HT}−d_{LT}. FIG. 20B shows relationships between the relative change in interlayer distance (Δd/d_{LT}) and the transition entropy (ΔSₜᵣ) associated with chain-melting transitions.

FIG. 21 Mixed halide perovskite with DA chain (n=10): volume change (ΔV) and and gravimetric entropy change (ΔS). Relationships between the relative change in interlayer distance (Δd/d_{LT}) and the transition entropy (ΔSₜᵣ) associated with chain-melting transitions for mixed-chain [(NA)₁₋ₓ(DA)ₓ]₂CuCl₄ perovskites.

FIGS. 22A-22D Mixed-chain/mixed-halide (“double-mixed”) perovskites: ambient-pressure PXRD for both HT and LT phases. FIGS. 22A-22D show powder X-ray diffraction (PXRD) patterns for compositionally engineered two-dimensional copper halide perovskites at 1 bar of He, with an X-ray wavelength of 0.45213 Å, for (FIGS. 22A and 12B) high-temperature (HT) and (FIGS. 22C and 12D) low-temperature (LT) phase. The PXRD patterns at HT phase and LT phase were obtained at 21 K above and below the transition temperature (Tₜᵣ), respectively. The PXRD patterns are shown at variable compositions (as indicated), with grey shades highlighting (002) and (003) reflections that are used to calculate the interlayer distance. The compounds contain nonylammonium (NA, C₉), decylammonium (DA, C₁₀), and/or undecylammonium (UA, C₁₁) chains confined within the Cu—X pocket (X=Cl, Br).

FIGS. 23A and 23B Mixed-chain/mixed-halide (“double-mixed”) perovskites: volume change and entropy changes. FIG. 23A shows interlayer distances for compositionally engineered two-dimensional copper halide perovskites for high-temperature (HT) and low-temperature (LT) phase. The data for HT (red) and LT (blue) phase were obtained at 21 K above and below the transition temperature (Tₜᵣ), respectively. The change in interlayer distance (Δd), indicated by a dashed arrow, is calculated as the difference between the interlayer distances for HT phase (d_{HT}) and LT phase (d_{LT}), with Δd=d_{HT}−d_{LT}. FIG. 23B shows relationships between the relative change in interlayer distance (Δd/d_{LT}) and the transition entropy (ΔSₜᵣ) associated with chain-melting transitions.

FIGS. 24A-24C show HP-DSC data for (NA)₂CuCl₄: FIG. 24A shows high-pressure differential scanning calorimetry measurements under applied hydrostatic pressure of helium for (NA)₂CuCl₄ with heating and cooling rates of 2 K min⁻¹. FIG. 24B shows pressure dependence of the major phase transition as determined by HP-DSC. FIG. 24C shows pressure dependence of the minor phase transition as determined by HP-DSC. The data points in the P-T diagram correspond to peak temperature. The minor transition has a noticeably lower entropy, and higher dT/dP and sensitivity to pressure. An “average” dT/dP of 22.1 K kbar⁻¹ was determined via the Clausius-Clapeyron relation.

FIGS. 25A and 25B show HP-DSC data for (DA)₂CuCl₂Br₂. FIG. 25A shows high-pressure differential scanning calorimetry measurements under applied hydrostatic pressure of helium for (DA)₂CuCl₂Br₂ with heating and cooling rates of 2 K min⁻¹. FIG. 25B shows pressure dependence of the phase transition as determined by HP-DSC, with peak transition temperatures plotted. A barocaloric coefficient (dT/dP) of 25.8 K kbar⁻¹ was determined from the heating curve.

FIGS. 26A and 26B show HP-DSC data for (DA)₂CuCl₃Br. FIG. 26A shows high-pressure differential scanning calorimetry measurements under applied hydrostatic pressure of helium for (DA)₂CuCl₃Br with heating and cooling rates of 2 K min⁻¹. FIG. 26B shows pressure dependence of the phase transition as determined by HP-DSC, with peak transition temperatures plotted. A barocaloric coefficient (dT/dP) of 24.3 K kbar⁻¹ was determined from the heating curve.

FIGS. 27A and 27B show HP-DSC data for [(NA)_{0.5}(DA)_{0.5}]₂CuCl₄. FIG. 27A shows high-pressure differential scanning calorimetry measurements under applied hydrostatic pressure of helium for [(NA)_{0.5}(DA)_{0.5}]₂CuCl₄ with heating and cooling rates of 2 K min⁻¹. FIG. 27B shows pressure dependence of the phase transition as determined by HP-DSC, with peak transition temperatures plotted. A barocaloric coefficient (dT/dP) of 22.4 K kbar⁻¹ was determined from the heating curve.

FIGS. 28A and 28B show HP-DSC data for [(NA)_{0.5}(UA)_{0.5}]₂CuCl₄. FIG. 28A shows high-pressure differential scanning calorimetry measurements under applied hydrostatic pressure of helium for [(NA)_{0.5}(UA)_{0.5}]₂CuCl₄ with heating and cooling rates of 2 K min⁻¹. FIG. 28B shows pressure dependence of the phase transition as determined by HP-DSC, with peak transition temperatures plotted. A barocaloric coefficient (dT/dP) of 25.1 K kbar⁻¹ was determined from the heating curve.

FIGS. 29A and 29B show HP-DSC data for [(NA)_{0.5}(DA)_{0.5}]₂CuCl₂Br₂. FIG. 29A shows high-pressure differential scanning calorimetry measurements under applied hydrostatic pressure of helium for [(NA)_{0.5}(DA)_{0.5}]₂CuCl₂Br₂ with heating and cooling rates of 2 K min⁻¹. FIG. 29A shows pressure dependence of the phase transition as determined by HP-DSC, with peak transition temperatures plotted. A barocaloric coefficient (dT/dP) of 24.1 K kbar⁻¹ was determined from the heating curve.

FIGS. 30A and 30B: Evaluation of barocaloric effects for (DA)₂CuCl₂Br₂. FIG. 30A shows DSC measurements for (DA)₂CuCl₂Br₂ under applied hydrostatic pressure with heating and cooling rates of 2 K min⁻¹ using He as the pressure-transmitting medium. FIG. 30B shows pressure-temperature (P, T) phase diagram determined from the isobaric HP-DSC experiments. Phase boundaries were determined for both heating (red) and cooling (blue), with the transition width highlighted in the shaded area. Note that the minimum pressure required to drive a reversible isothermal entropy change (Pᵣₑᵥ, 31 bar) and a reversible adiabatic temperature change (P_{rev,ad}, 130 bar) are indicated by vertical lines.

FIGS. 31A-31D: Entropy curves for (DA)₂CuCl₂Br₂. FIGS. 31A and 31B show isobaric entropy change (ΔS_{ib}) associated with the phase transition of a powder sample of (DA)₂CuCl₂Br₂, as a function of temperature in the pressure range of 1 bar to 150 bar on (FIG. 31A) heating and (FIG. 31B) cooling. FIGS. 31C and 31D shows isothermal entropy changes (ΔSᵢₜ), calculated by the quasi-direct method, for (FIG. 31C) decompression to ambient pressure and (FIG. 31D) compression from ambient pressure, obtained from heating and cooling data, respectively.

FIGS. 32A and 32B: Evaluation of barocaloric effects for (DA)₂CuCl₂Br₂. FIG. 32A shows isothermal entropy changes (ΔSᵢₜ) calculated by the quasi-direct method for (DA)₂CuCl₂Br₂. The shaded area indicates the reversible isothermal entropy change (ΔS_{it,rev}) that is accessible at each operating pressure. FIG. 32 shows the maximum reversible isothermal entropy change (ΔS_{it,rev,max}) and reversible refrigeration capacity (RCᵣₑᵥ) are plotted as a function of operating pressure. Note that the barocaloric strength (ΔS_{it,rev}/ΔP) is 1391 J K⁻¹ kg⁻¹ kbar⁻¹ and the pressure dependence of RCᵣₑᵥ is 2723 J K⁻¹ kg⁻¹ kbar⁻¹.

FIGS. 33A and 33B: Phase Diagram for (NA_{0.5}DA_{0.5})₂CuCl₂Br₂. FIG. 33A shows DSC measurements for (NA_{0.5} DA_{0.5})₂CuCl₂Br₂ under applied hydrostatic pressure with heating and cooling rates of 2 K min⁻¹ using He as the pressure-transmitting medium. FIG. 33B shows a pressure-temperature (P, T) phase diagram determined from the isobaric HP-DSC experiments. Phase boundaries were determined for both heating (red) and cooling (blue), with the transition width highlighted in the shaded area. Note that the minimum pressure required to drive a reversible isothermal entropy change (Pᵣₑᵥ, 21 bar) is indicated by a vertical line. Note that P_{rev,ad} is 160 bar.

FIGS. 34A-34D: Entropy curves for (NA_{0.5} DA_{0.5})₂CuCl₂Br₂. FIGS. 34A and 34B show isobaric entropy change (ΔS_{ib}) associated with the phase transition of a powder sample of (NA_{0.5}DA_{0.5})₂CuCl₂Br₂, as a function of temperature in the pressure range of 1 bar to 150 bar on (FIG. 34A) heating and (FIG. 34B) cooling. FIGS. 34C and 34D show isothermal entropy changes (ΔSᵢₜ), calculated by the quasi-direct method, for (FIG. 34C) decompression to ambient pressure and (FIG. 34D) compression from ambient pressure, obtained from heating and cooling data, respectively.

FIGS. 35A and 35B: Evaluation of barocaloric effects for (NA_{0.5}DA_{0.5})₂CuCl₂Br₂. FIG. 35 shows isothermal entropy changes (ΔSᵢₜ) calculated by the quasi-direct method for (NA_{0.5}DA_{0.5})₂CuCl₂Br₂. The shaded area indicates the reversible isothermal entropy change (ΔS_{it,rev}) that is accessible at each operating pressure. FIG. 35B shows the maximum reversible isothermal entropy change (ΔS_{it,rev,max}) and reversible refrigeration capacity (RCᵣₑᵥ) are plotted as a function of operating pressure. Note that the barocaloric strength (ΔS_{it,rev}/ΔP) is 1085 J K⁻¹ kg⁻¹ kbar⁻¹ and the pressure dependence of RCᵣₑᵥ is 1626 J K⁻¹ kg⁻¹ kbar⁻¹.

FIG. 36 Energy-dispersive X-ray spectroscopy measurements and scanning electron microscope (SEM) for (DA)₂CuCl₃Br. The SEM image is shown on the left. Elemental maps of Br (top right) and Cl (bottom right) show an even dispersion of halides on the crystal surface. The magnification is 1,300× and the electron accelerating voltage is 4.0 kV. These results show that Cl and Br are uniformly mixed in the crystal.

FIGS. 37A and 37B show halide quantification from elemental analysis. Molar bromide content for (NA)₂CuCl₄₋ₓBrₓ (FIG. 37A) and (DA)₂CuCl₄₋ₓBrₓ (FIG. 37B) was found through elemental analysis. These percentages were determined by oxygen flask combustion and potentiometric titration. The lines delineate what the bromide content would be if all of the reagents in solution were incorporated into the crystal structure. Deviation from expected concentration is likely due to solubility differences between bromide and chloride perovskites. All mixed-halide/chain compounds listed herein are referred to by their expected (nominal) molar ratios (i.e., concentration in solution) for the sake of consistency.

FIG. 38 shows a depiction of how dialkylammonium (organic) salts can be used to drive a barocaloric cooling cycle. The order-disorder transitions in the organic bilayers are pressure-dependent. First, an adiabatic compression induces a transition from the expanded, disordered phase of the material to a contracted, ordered phase. Heat that is released during this exothermic process is rejected to a heat sink, returning the material to its original temperature but at a lower entropy. Then, the pressure is adiabatically decreased to reverse the phase transition and take in heat from a heat source.

FIG. 39 Structural characterization of (C₆H₁₃)₂NH₂Br. XRD experiments were performed on (C₆H₁₃)₂NH₂Br. The single-crystal structure obtained at 100 K (left, viewed along b-axis) features a layered structure, where each organic cation is confined by charge-balancing Br anions. We have also successfully carried out variable-temperature PXRD experiments under 300 bar of He (right). The data was collected during cooling from 360 K to 220 K, with an X-ray wavelength of 0.45185 Å. Notably, these data show that, even at 300 bar pressure, the compound undergoes a single (disorder-to-order) phase transition that is associated with a large change in volume.

FIG. 40 Structural characterization of (C₁₂H₂₅)(CH₃)NH₂Br “(C₁₂-C₁)Br”. XRD experiments were performed on (C₁₂H₂₅)(CH₃)NH₂Br. The single-crystal structure obtained at 100 K (left, viewed along b-axis) features a layered structure, where each organic cation is confined by charge-balancing Br anions.

FIG. 41 Structural characterization of (C₁₂H₂₅)(CH₃)NH₂Cl “(C₁₂-C₁)Cl”. We have carried out preliminary XRD experiments on (C₁₂H₂₅)(CH₃)NH₂Br. The single-crystal structure obtained at 100 K (left, viewed along b-axis) features a layered structure, where each organic cation is confined by charge-balancing Br anions.

FIGS. 42A and 42B show data from high-pressure differential scanning calorimetry for (C₁₂-C₁)Br. FIG. 42A shows DSC measurements for (C₁₂H₂₅)(CH₃)NH₂Br “(C₁₂-C₁)Br” under applied hydrostatic pressure with heating and cooling rates of 2 K min⁻¹ using He as the pressure-transmitting medium. FIG. 42B shows a pressure-temperature (P, T) phase diagram determined from the isobaric HP-DSC experiments. Phase boundaries were determined for both heating and cooling.

FIGS. 43A and 43B show data from high-pressure differential scanning calorimetry for (C₁₂-C₁)Cl. FIG. 43A shows DSC measurements for (C₁₂H₂₅)(CH₃)NH₂Cl “(C₁₂-C₁)Cl” under applied hydrostatic pressure with heating and cooling rates of 2 K min⁻¹ using He as the pressure-transmitting medium. FIG. 43B shows a pressure-temperature (P, T) phase diagram determined from the isobaric HP-DSC experiments. Phase boundaries were determined for both heating and cooling.

FIGS. 44A and 44B show data from high-pressure differential scanning calorimetry for dC₆Br. FIG. 44A shows DSC measurements for (C₆H₁₃)₂NH₂Br “dC₆Br” under applied hydrostatic pressure with heating and cooling rates of 2 K min⁻¹ using He as the pressure-transmitting medium. FIG. 44B shows a pressure-temperature (P, T) phase diagram determined from the isobaric HP-DSC experiments. Phase boundaries were determined for both heating and cooling.

FIG. 45 shows thermal properties of organic barocaloric materials for a selected group of dialkylammonium salt compounds (left). Entropy changes associated with the order-disorder phase transition (measured by ambient-pressure DSC) are plotted (right) as a function of transition temperatures. Importantly, these materials undergo reversible phase transitions accompanied by colossal entropy changes (>200 J K⁻¹ kg⁻¹) near ambient temperature.

FIGS. 46A and 46B show pressure dependence of phase transition temperature is highly sensitive to the pressure-transmitting medium. Pressure-temperature (P, T) phase diagram determined under He (red), N₂, and Ar environments for (FIG. 46A) (DA)₂MnCl₄ and (FIG. 46B) (NA)₂CuBr₄ via high-pressure differential scanning calorimetry (HP-DSC). The pressure sensitivity of the order-disorder phase transition (dT/dP) decreases as the polarizability of gas medium increase (from He to N₂ to Ar). The phase boundaries and dT/dP values predicted from the Clausius-Clapeyron relationship (dT/dP=ΔV/ΔS) are shown in diamonds.

FIGS. 47A and 47B show entropy of transitions measured from isobaric HP-DSC experiments under He, N₂, and Ar for (FIG. 47A) (DA)₂MnCl₄ and (FIG. 47B) (NA)₂CuBr₄. The identity of pressure-transmitting medium has minimal impact on the magnitudes of transition entropy.

FIGS. 48A and 48B show results for how Ar gas as pressure-transmitting medium induces inverse barocaloric effects in (NA)₂CuBr₄. FIG. 48A shows isobaric high-pressure differential scanning calorimetry (HP-DSC) measurements for (NA)₂CuBr₄ under Ar environments reveal that the increase in Ar pressure leads to decrease in transition temperature, without noticeable changes in transition enthalpy and entropy. FIG. 48B is a (P, T) phase diagram under Ar, with transition temperatures determined from heating and cooling.

FIGS. 49A and 49B show direct evaluation of Ar pressure-induced inverse barocaloric effects in (NA)₂CuBr₄ via quasi-isothermal HP-DSC. FIG. 49A shows a pressure-temperature phase diagram. In FIG. 49B heat flow signals were measured as a function of time during 3 cycles of applying and removing a hydrostatic pressure of 150 bar at 301.6 K with Ar as the pressure-transmitting medium. As the phase diagram shown in FIG. 49A indicates, the 150-bar pressure swing at 301.6 K, compression induces an endothermic transition from ordered (low-temperature, LT) phase to disordered (high-temperature, HT) phase. Decompression induces an exothermic transition from disordered/HT phase to ordered/LT phase. The area under the heat flow peaks in FIG. 49B correspond to compression-induced endotherms and decompression-induced exotherms is 23.3 and 22.5 J/g, respectively.

FIG. 50 shows how manipulating the pressure sensitivity (dT/dP) via pressure-transmitting medium provides a mechanism for pressure-tunable thermal energy storage. Transition temperatures for (NA)₂CuBr₄ can be increased to 309 K (at increasing He pressure) or lowered to 300 K (at increasing Ar pressure), which creates the temperature window of 9 K at 150 bar pressure. This phenomenon can be utilized for tuning the transition temperatures “on-demand” for thermal energy storage at easily accessible pressure. Thermal energy storage (TES) cycles at low temperatures can be realized under high-pressure Ar environments, whereas TES cycle at high temperatures can be accessed under high-pressure He.

### DETAILED DESCRIPTION OF THE INVENTION

Barocaloric effects—thermal changes driven by hydrostatic pressure—offer particularly simple and energy-efficient ways to achieve solid-state cooling. As first-order phase transitions involve a latent heat, large barocaloric effects are expected to occur near the phase transition temperature when the first-order transition is induced by applied hydrostatic pressure. To exhibit a large barocaloric effect, a material must meet the following three requirements: 1) first-order phase transition with large latent heat (Gₜᵣ), large entropy change (ΔSₜᵣ), and transition temperature Tₜᵣ close to a desired operational temperature; 2) high sensitivity of the phase transition to applied pressure (i.e., high barocaloric coefficient δTₜᵣ/δP); and, 3) low thermal hysteresis. As cooling devices operate cyclically, the barocaloric effects must be driven reversibly upon a sequence of application and removal of external pressure; therefore, thermal hysteresis dramatically affects the cooling performance, determining the minimum pressure needed to achieve reversible barocaloric effects (pᵣₑᵥ). Usually, pᵣₑᵥ is proportional to the thermal hysteresis width. Since it is practically beneficial to have low operating pressure (pᵣₑᵥ), identifying barocaloric materials with low thermal hysteresis is highly desirable. Note, additionally, that for a first-order phase transition, the barocaloric coefficient can be calculated using the Clausius-Clapeyron relation δTₜᵣ/δP=ΔVₜᵣ/ΔSₜᵣ where ΔVₜᵣ=volume change of the phase transition. Thus, a material must have a high ΔVₜᵣ/ΔSₜᵣ to have a high barocaloric coefficient.

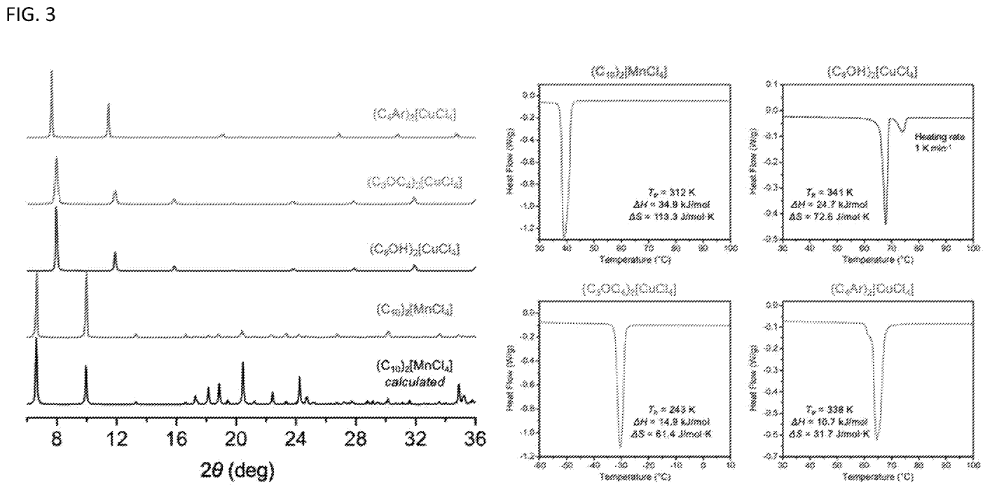

The invention provides highly generalizable approaches to realizing a new class of materials that display colossal—yet tunable—barocaloric effects at relatively low operating pressures. Specifically, this invention describes the use of reversible, solid-solid, order-disorder phase transitions, e.g., in layered, two-dimensional (2D) organic-inorganic hybrid materials for barocaloric cooling (FIG. 3A). In these newly identified hybrid barocaloric compounds, long-chain organic molecules (e.g., long alkyl chain CₙH₂ₙ₊₁, e.g., where n>3, e.g., >4) are templated by inorganic layers, and the hybrid materials exhibit pressure-sensitive phase transitions based on order-disorder transitions of the confined chains—a so-called “chain-melting” process. We believe that any phase-change material for which a solid-state phase transition results from a structural transitions of long-chain organic molecules confined within inorganic components is likely to exhibit large barocaloric effects.

The propensity for confined chain melting to drive large barocaloric effect is based on: (i) the wide range of organic phase-change materials that undergo phase transitions near ambient temperature with a large volume change (ΔVₜᵣ), latent heat (Qₜᵣ), and entropy change (ΔSₜᵣ); (ii) the organic phase-change materials, when templated with inorganic substances, remain solid-state during the phase change, exhibiting reversible, solid-solid phase transitions with all beneficial phase-change properties (i.e., high ΔVₜᵣ, Qₜᵣ, ΔSₜᵣ) retained. Unlike magnetocaloric, electrocaloric, and elastocaloric effects, the barocaloric effect is not system-selective and in principle can be observed in any materials, as the free energy of a system always depends on pressure. In summary, this invention reports that order-disorder transition of normal, branched, or functionalized organic chains—on or within inorganic structural templates—can be used as a new mechanism to achieve large barocaloric effects relevant to solid-state cooling.

Layered materials such as two-dimensional (2-D) metal-halide perovskites of the form (R-NH₃)₂MX₄ (R=CₙH₂ₙ₊₁; n>3 (e.g., >4); M=Mn, Fe, Cu, Cd, Pb; X=F, Cl, Br, or I) can undergo chain-melting transitions in the solid state. In these compounds, sheets of corner-sharing MX₆ octahedra create anionic pockets-defined by the axial halides of four adjacent metal centers—that template the arrangement of bilayers of alkylammonium cations through charge-assisted hydrogen bonds. When long-chained hydrocarbon molecules (n>3, e.g., >4) are incorporated, many layered perovskites undergo thermally induced, first-order phase transitions between ordered and disordered states driven by what is effectively a partial melting transition of the hydrocarbon bilayers. As such 2-D perovskites can serve as a highly tunable solid-state platform to leverage the large changes in entropy and enthalpy that accompany hydrocarbon chain-melting transitions for barocaloric cooling (FIGS. 3A-3B). Moreover, since the inorganic layers and organic bilayers of 2-D perovskites can be independently manipulated, phase transition hysteresis could be minimized through careful control of the organic-inorganic interfaces.

In one embodiment, this invention describes barocaloric effects in 2D hybrid perovskites, with a general chemical formula of (R-NH₃)₂MX₄, which contain layers of first-row transition metal halides [MX₄]²⁻ (M=Mn, Fe, Co, Cu, Zn; X=Cl, Br, or I) connected by bilayers of ammonium cations (R-NH₃⁺).

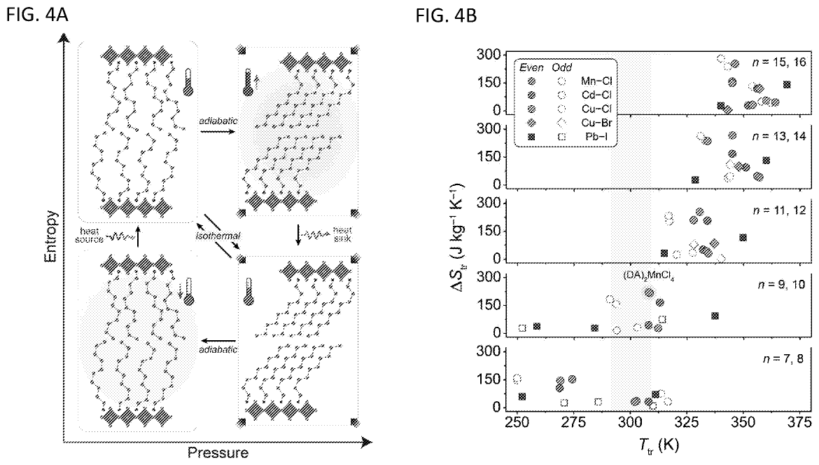

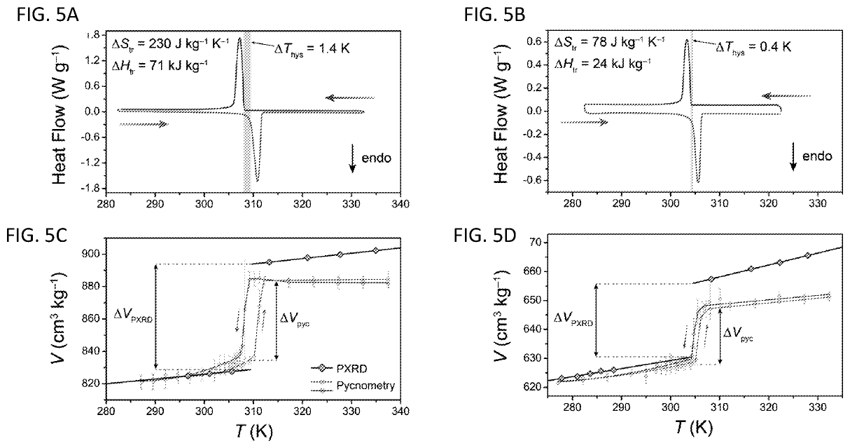

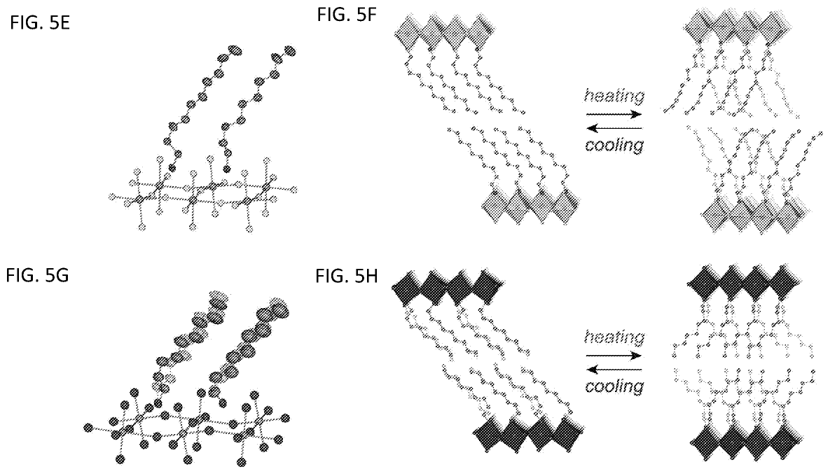

In these compounds, organic bilayers are confined by metal-halide inorganic layers. Inorganic layers can assemble (and confine organic layers) via, e.g., hydrogen bonds (e.g., between R-NH₃⁺ . . . X⁻ groups), electrostatic attraction, or a combination thereof. Van der Waals interactions between R groups (see, e.g., FIGS. 3A and 5E-5H) may also contribute to the assembly of layers and confinement of the organic layers. Depending on the identity of the divalent transition metal cation (M²⁺), the inorganic layers of the 2D layered perovskites have two different structure types. For M=Mn, Fe, Cu, the inorganic layer is composed of corner-sharing MX₆ octahedra. Each organic molecule is contained within a cavity defined by the terminal halides from four adjacent corner-sharing octahedra. For M=Co, Zn, the inorganic layer consists of planes of isolated MX₄ tetrahedra. In all cases, the bilayers of organic chains (R-NH₃⁺) are confined by the inorganic layers.

Many 2D perovskites with long-chain organic molecules (R=CₙH₂ₙ₊₁, n>4) are known to undergo thermally-induced, reversible, solid-solid phase transitions near room temperature (20-90° C.) due to the ordering and disordering the organic molecules. As these solid-solid transitions are often accompanied by a large latent heat (>60 kJ kg⁻¹) and entropy change (>200 J kg⁻¹ K⁻¹), layered hybrid perovskites may be employed as solid-solid phase-change materials for passive thermal management and thermal energy storage.

The main phase transition of these compounds involves disordering of organic chains, often referred to as a “chain-melting” transition. The mechanism for “chain melting”—defined as the rapid diffusion of a kink (one or more gauche bonds) up and down along the C—C bonds within an organic chain—has been extensively studied at ambient pressure by various structural, thermal, and spectroscopic techniques, including powder and single-crystal X-ray diffraction, differential scanning calorimetry, dielectric measurements, and infrared and Raman spectroscopies. The dynamics of the ammonium chains have been further investigated at ambient pressure by solid-state NMR techniques and inelastic neutron scattering. In the low-temperature, ordered phase, the confined chains are tilted because their cross-sectional area is less than the area of the halide square (˜5×5 Å²) afforded by the 2D inorganic lattice.

In the high-temperature, disordered phase, the chains are disordered and effectively occupy the whole cross-sectional area available to them. This results in a large expansion of the interlayer spacing. As the intralayer distances are essentially unchanged due to the robust inorganic templates, this directly leads to large increase in volume (ΔVₜᵣ/V₀=7-10%). These studies show that the large latent heat, entropy change, and volume change of main phase transition observed in the organic-inorganic hybrid materials originate from confined chain melting process.

A representative layered perovskite (C₁₀H₂₁NH₃)₂MnCl₄ undergoes solid-solid, reversible phase transition near room temperature (35° C.) with large entropy change (e.g., ˜221 J kg⁻¹ K⁻¹) and volume expansion (e.g., ˜7.3%). Based on thermodynamics calculations (Clausius-Clapeyron relation), we identified that the phase transition is expected to be highly sensitive to applied pressure (e.g., δTₜᵣ/δP˜28 K kbar⁻¹). As such, inducing the disorder-to-order transition by adiabatically applying pressure is expected to result in adiabatic temperature increase, e.g., (ΔT_{ad}) of ˜30 K. Taken together, this analysis suggested that (C₁₀H₂₁NH₃)₂MnCl₄ should exhibit colossal barocaloric effect.

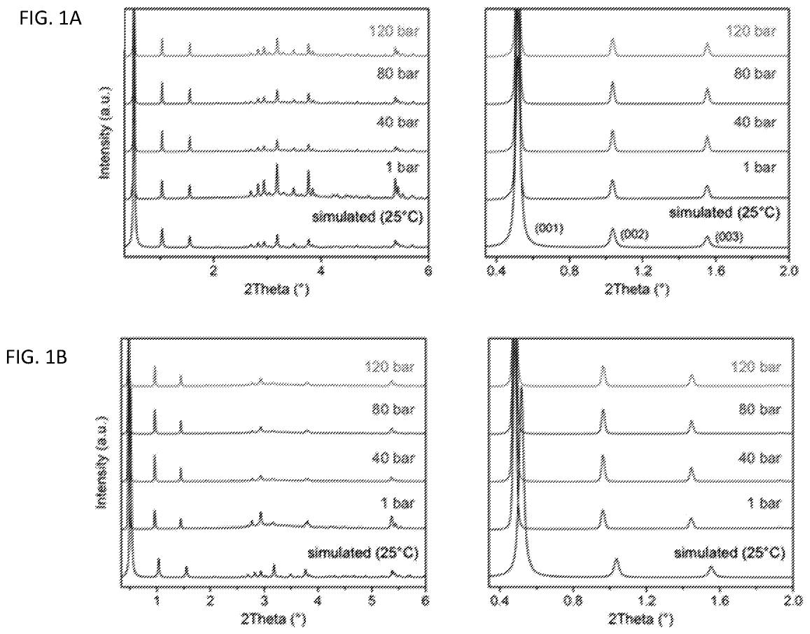

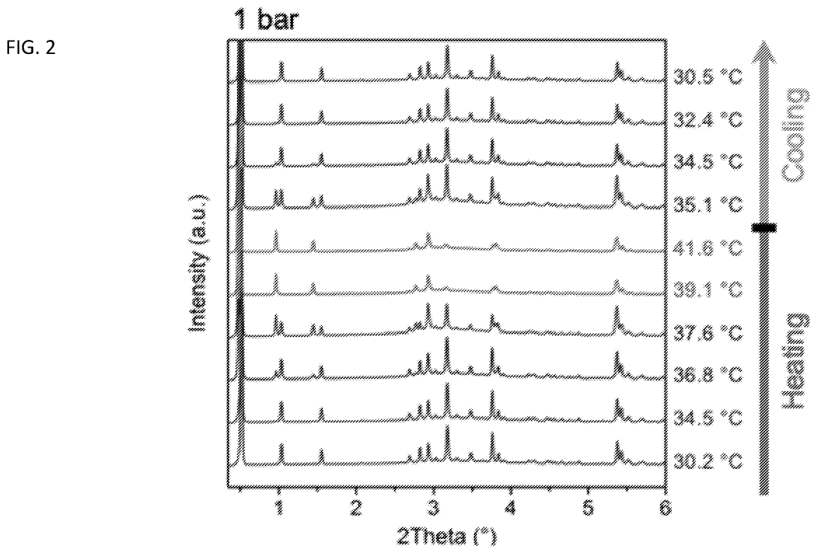

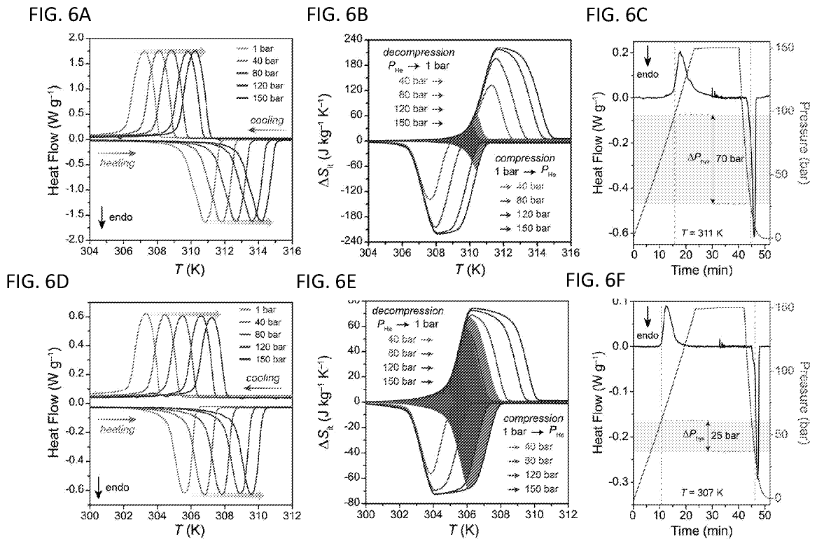

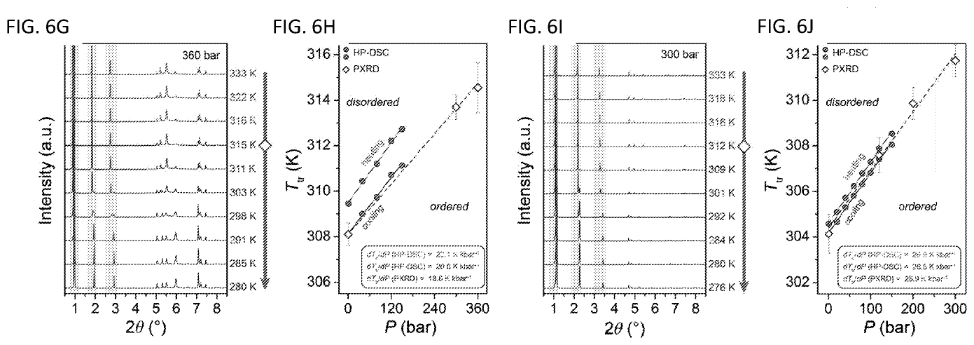

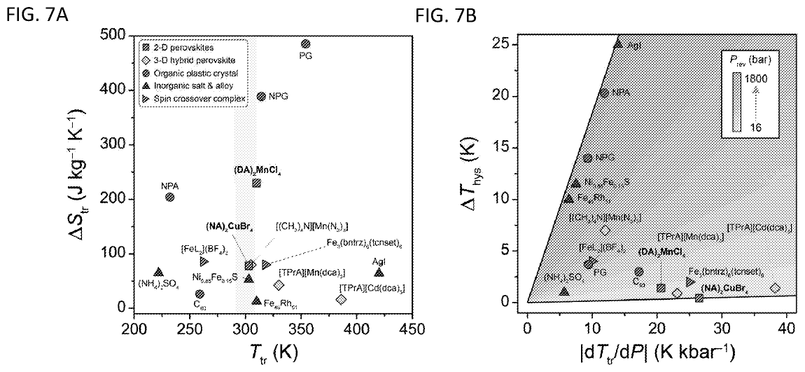

To experimentally confirm the existence of large barocaloric effects in layered perovskites, we measured powder X-ray diffraction (PXRD) patterns as a function of temperature and applied pressure for the 2D layered perovskites (C₁₀H₂₁NH₃)₂MnCl₄ to evaluate the pressure dependence of the transition temperature (Tₜᵣ) between ordered and disordered phases of this material. As the thermally induced order-disorder phase transition leads to a clear shift in powder diffractions peak positions (FIGS. 1A-1B), we were able to identify Tₜᵣ as a function of applied pressure. Excitingly, variable-temperature PXRD data obtained under applied pressures up to 360 bar clearly indicate that applying hydrostatic pressure shifts the phase transition to higher temperature (FIGS. 2 and 6A-6J). Compositions of the invention thus exhibit colossal barocaloric effects and are very competitive with the best current barocaloric materials (see, e.g., FIGS. 7A and 7B). More broadly, the results described herein indicate that the thermodynamic and structural properties afforded by confined chain melting transitions are generally beneficial for barocaloric effects.

The class of existing and potential 2D layered perovskites provide access to a tremendous structural and chemical diversity through the judicious selection of the inorganic and organic moieties that constitute each material. As such, we may control the confined chain melting in these materials leading to not only colossal but also highly tunable barocaloric effects. Indeed, the thermodynamics and kinetics of these pressure-induced phase transitions are controllable, e.g., by modifying: 1) the molecular interactions between the inorganic layers, 2) the flexibility of the organic chains, and 3) the free volume within the organic bilayers.

For instance, we have synthesized a series of new layered perovskites—containing Cu, Mn, and Fe metal centers ligated to Cl⁻ anions—with organic molecules incorporated as bilayers between the metal-chloride sheets that include oxygen-substituted alkyl chains (C₃OC₄), functionalized phenylalkylamines (C₄Ph and C₆Ph), alkyl chains incorporating an ester group (Cₙ—COO—C₂; n=9, 10, 11), and alkyl chains functionalized with alcohols (CₙOH; n=5, 6, 8) that create hydrogen bonding networks within the bilayers (see, e.g., FIG. 3 and Table 1 and Tables 20-25).

| | | | | ΔSₜᵣₐₙₛᵢₜᵢₒₙ | ΔSₜᵣₐₙₛᵢₜᵢₒₙ | | |
|---|---|---|---|---|---|---|---|
| | | Compound | Tₜᵣ (K) | (J kg⁻¹ K⁻¹) | (J L⁻¹ K⁻¹) | Qₜᵣ (kJ L⁻¹) | |
| C [symbol] OC₄ | 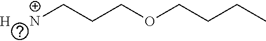 | [TPrA][Mn(dca]₃] | 330 | 42.5 | 52.7 | 17.3 | |
| C [symbol] | 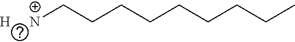 | (CH₃)₂C(CH₂OH)₂ | 313 | 390 | 390.0 | 122.1 | |
| C₁₀ | 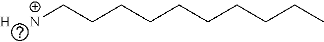 | (C [symbol] ) [symbol] Mn | 287 | 219.7 | 263.6 | 73.9 | Simple hydrocarbon chains |
| C₁₁ | 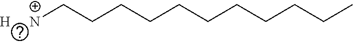 | (C₁₀) [symbol] Mn | 308 | 220.7 | 264.8 | 82.1 | |
| C₁₀(COO)C₂ | 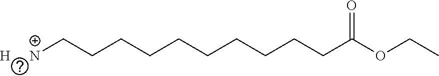 | (C₁₀)₂Fe | 308/311 | 215.5 | 274.4 | 84.2 | |
| C [symbol] (COO)C₂ | 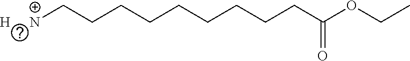 | (C₁₀)₂Cu | 309/312 | 244.4 | 293.3 | 90.6 | |
| C₁₁(COO)C [symbol] | 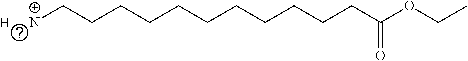 | (C₁₁)₂Mn | 316 | 270.6 | 324.7 | 103.1 | |
| C₄(C [symbol] F [symbol] ) | 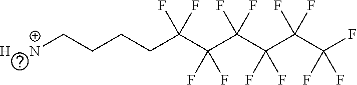 | (C₁₂)₂Mn | 332/336 | 285 | 342 | 118.1 | |
| C₃O(C [symbol] F [symbol] ) | 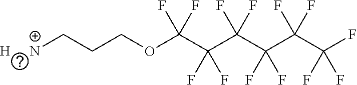 | (C₁₂)₂Cu | 328/334 | 252.9 | 303.5 | 95.3 | |
| C₄Ph | 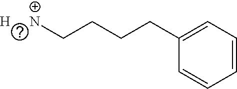 | (C [symbol] OC₄) [symbol] Cu | 243 | 130.7 | 183.0 | 44.5 | Functionalized chains |
| C [symbol] OH | 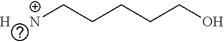 | (C [symbol] Ar)₂Cu | 338 | 62.7 | 70.8 | 23.9 | |
| C [symbol] OH | 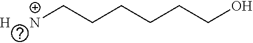 | (C [symbol] OAr) [symbol] Cu | 396 | 134 | 162 | 64 | |
| C [symbol] OH | 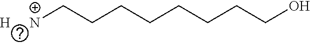 | (C [symbol] OH)₂Cu | 309 | 105.9 | 148.3 | 45.8 | |
| C [symbol] OC₄OH | 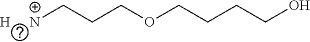 | (C [symbol] OH) [symbol] Cu | 341 | 145.8 | 204.1 | 69.6 | |
| C [symbol] OC [symbol] OH | 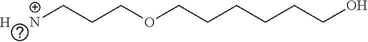 | (C [symbol] OH) [symbol] Mn | 342 | 223.8 | 305.0 | 104.2 | |
| C [symbol] OPh | 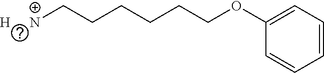 | [C₁₁(COO)C₂]₂Cu | 356 | 252.7 | 331.0 | 117.5 | |

[symbol]

indicates data missing or illegible when filed

Table 1 shows a library of long-chain ammonium cations incorporated into layered perovskites. Structures of long-chain ammonium cations studied in our laboratory (left) and thermal properties of layered perovskites incorporating the ammonium cation chains (right). Tₜᵣ, transition temperature; ΔSₜᵣₐₙₛᵢₜᵢₒₙ, entropy of phase transition; Qₜᵣ, latent heat of phase transition. (R)₂M denotes layered perovskite (R-NH₃)₂MCl₄.

These newly synthesized 2D hybrid perovskites exhibit reversible, thermally-induced phase transitions driven by chain melting. Most notably, the synthetic modifications of the organic chain enabled the tuning of transition temperature between −30° C. to 120° C. without compromising beneficial thermodynamic properties (e.g., large latent heat and entropy change). These results demonstrate that the barocaloric properties of layered perovskites can be readily tuned through synthetic modifications. Barocaloric effects of the functionalized perovskites synthesized in our laboratory are summarized in Table 5.

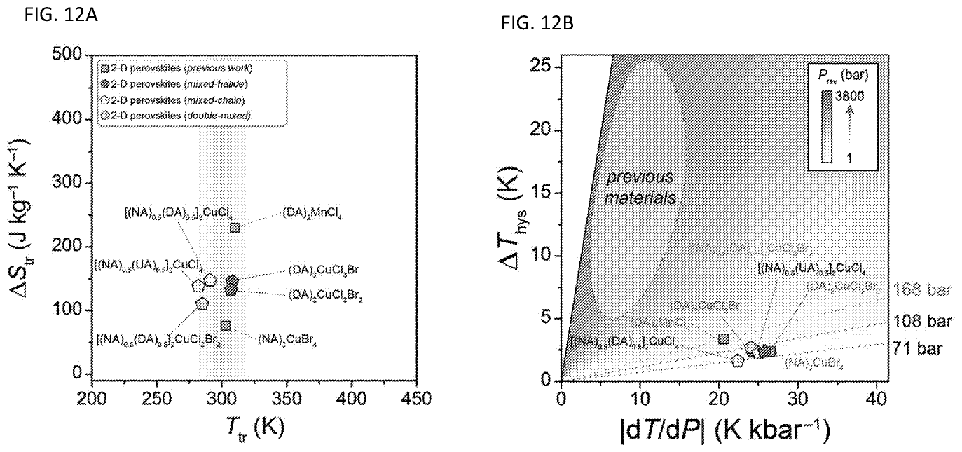

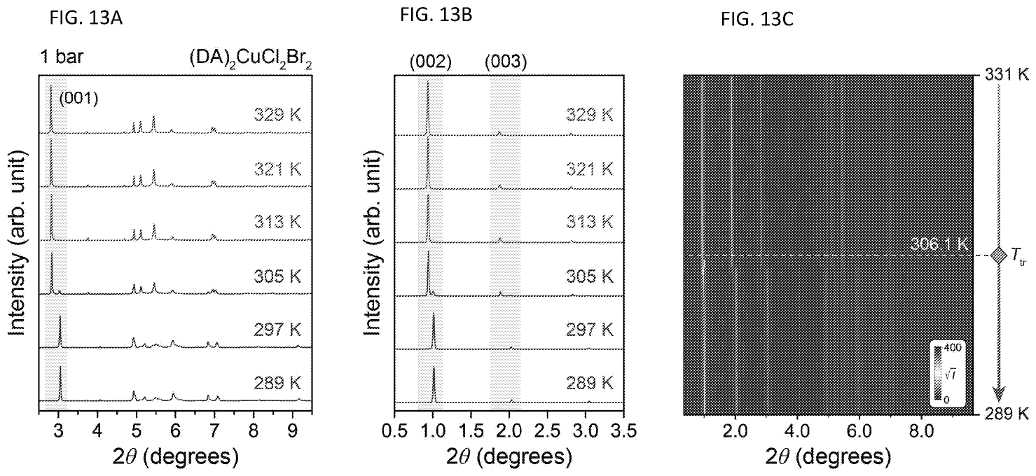

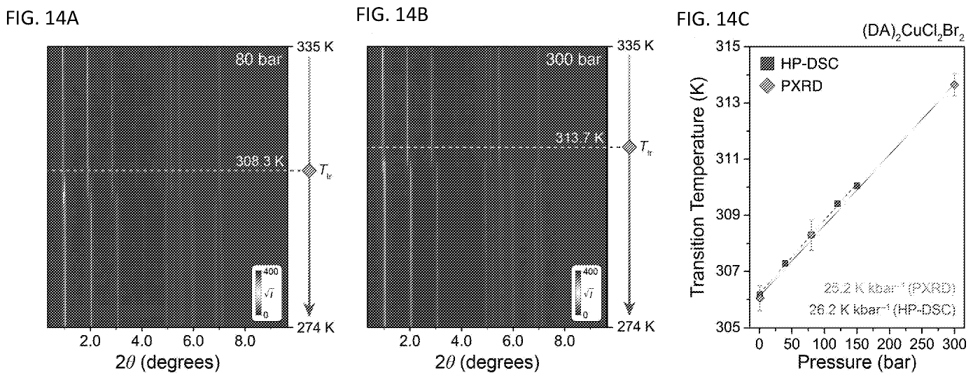

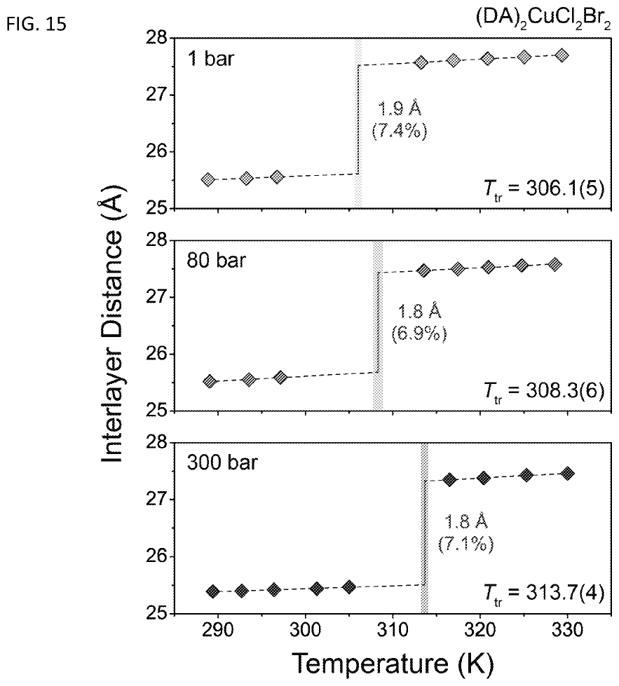

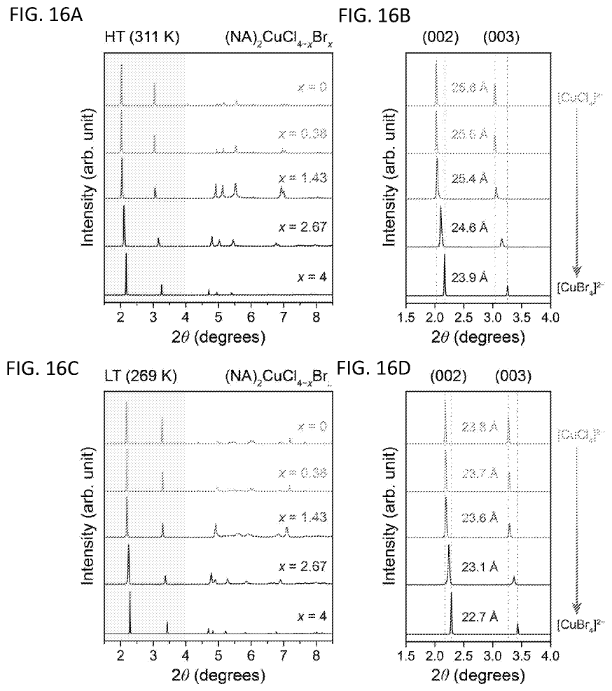

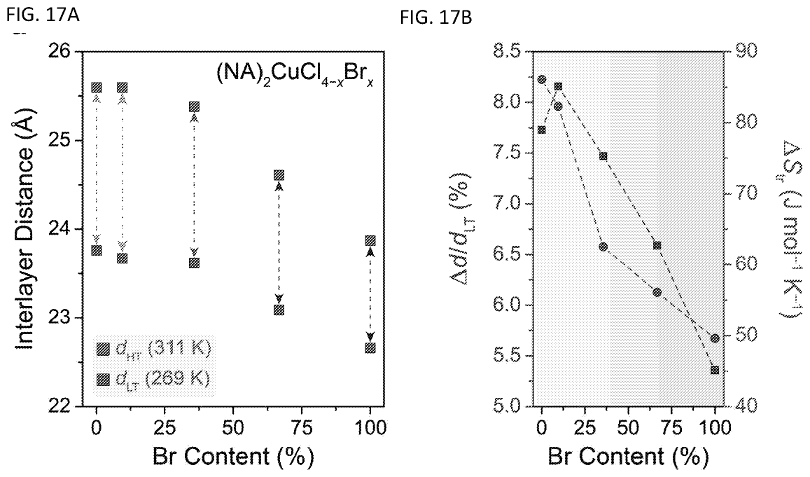

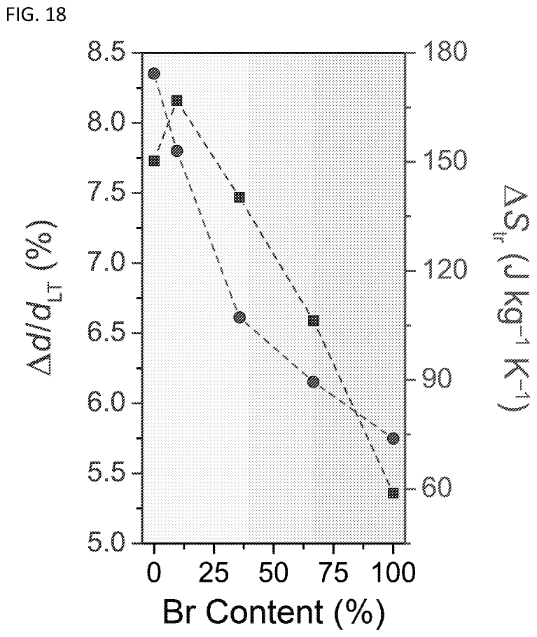

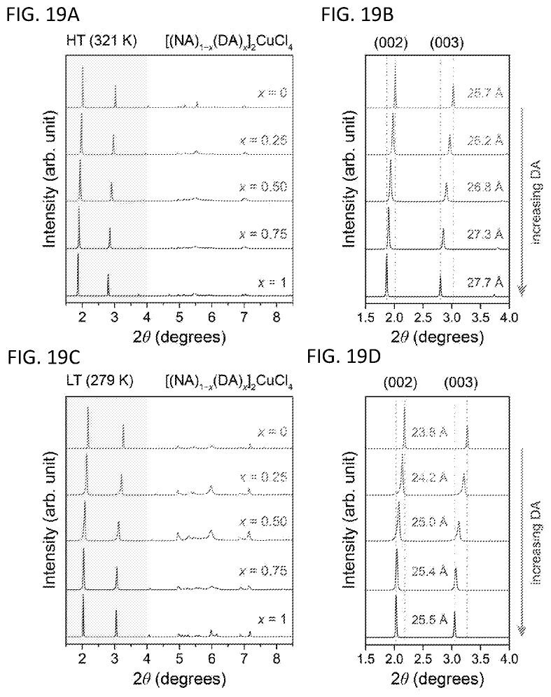

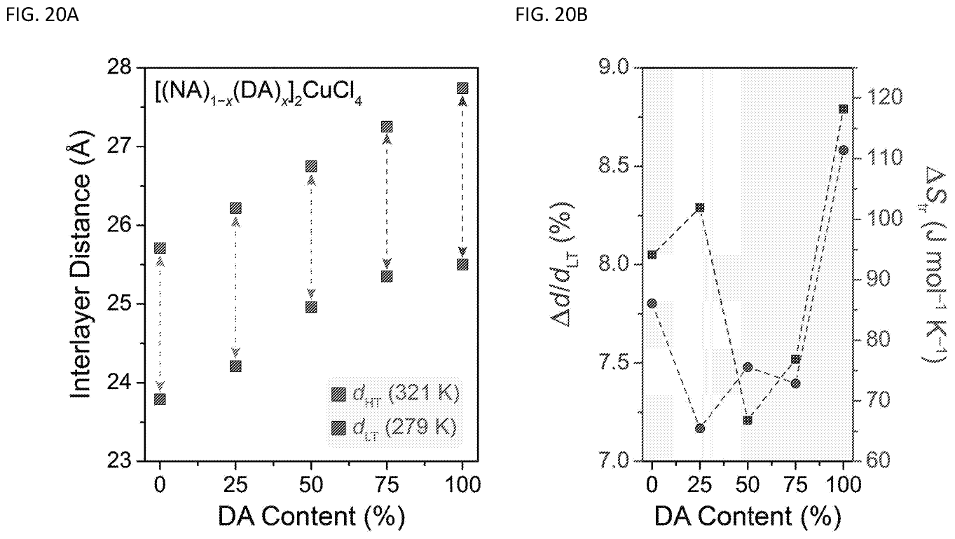

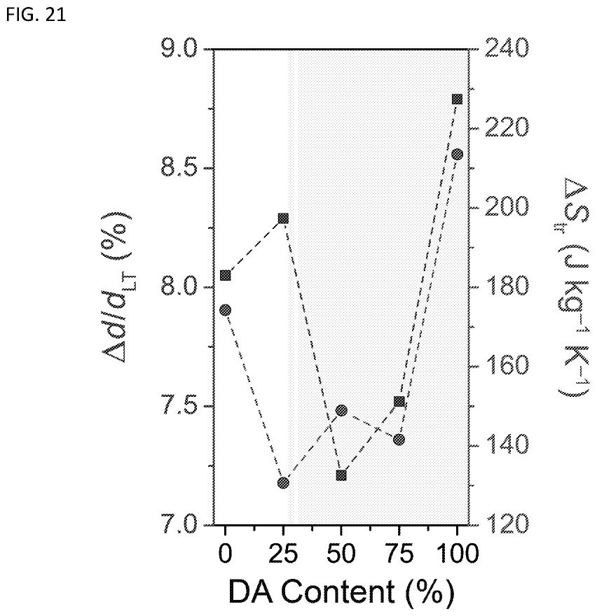

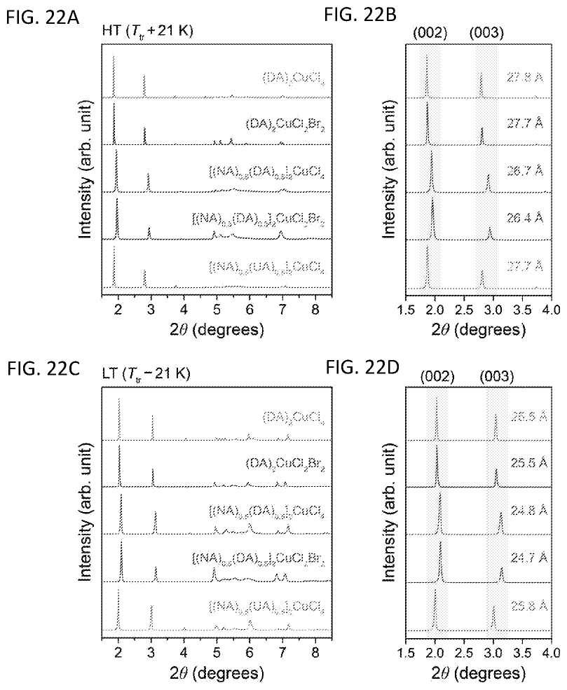

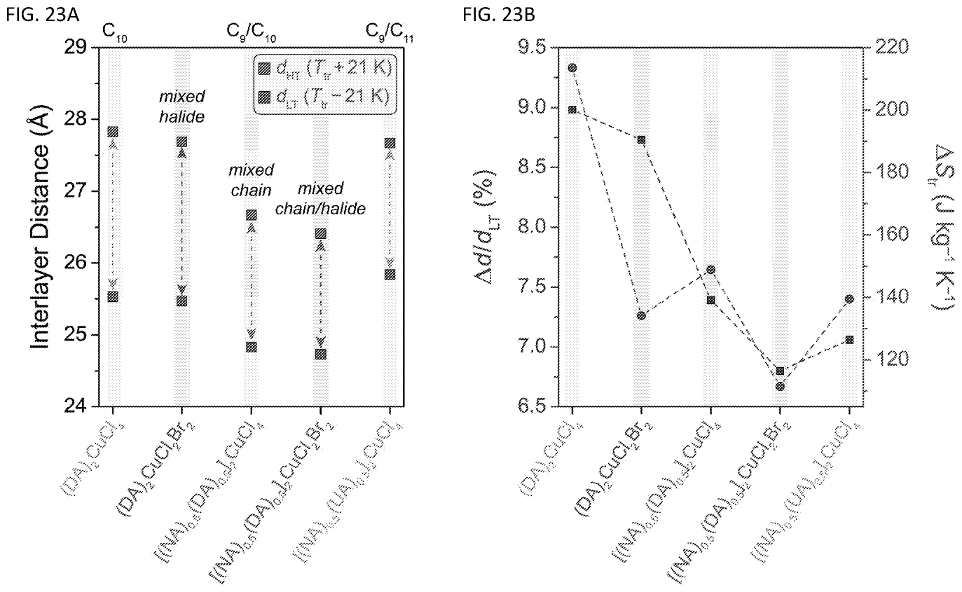

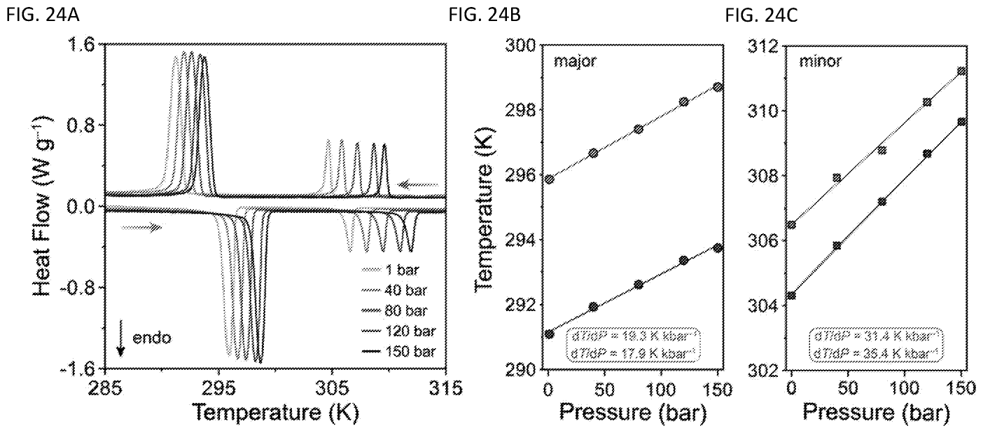

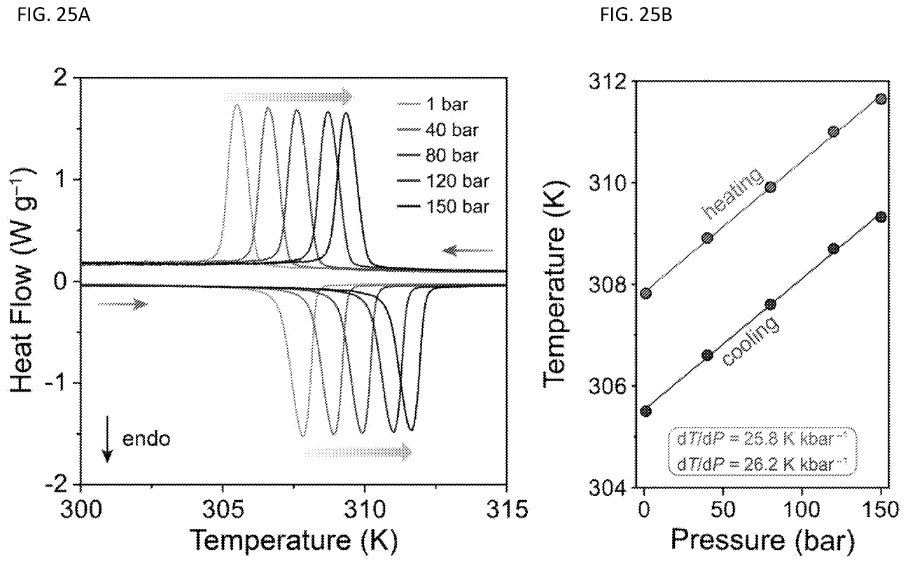

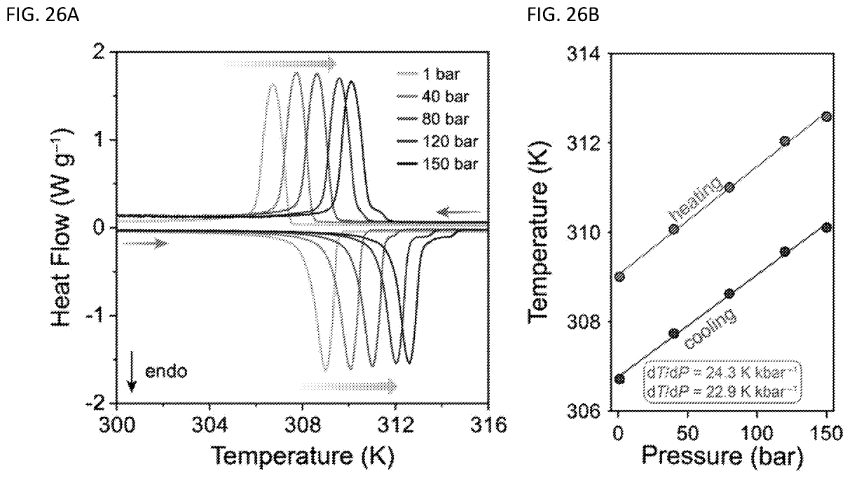

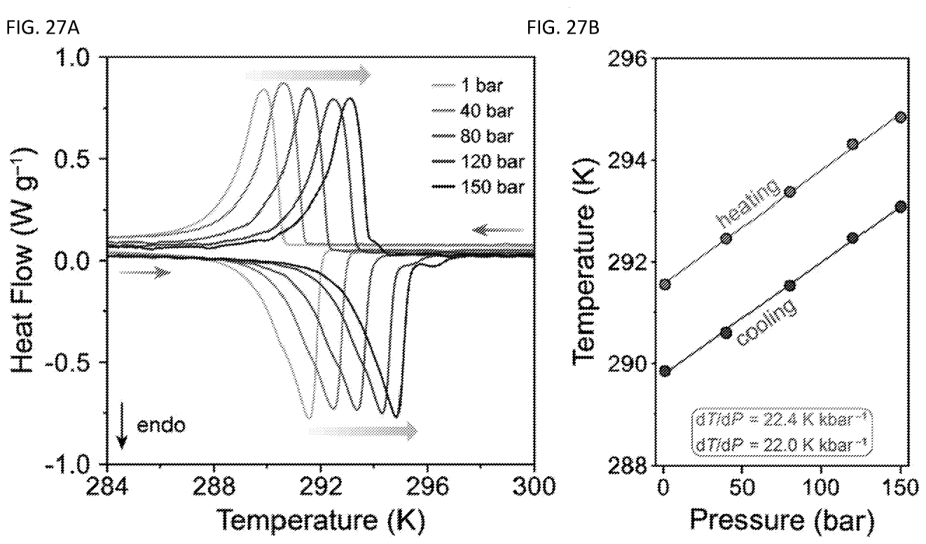

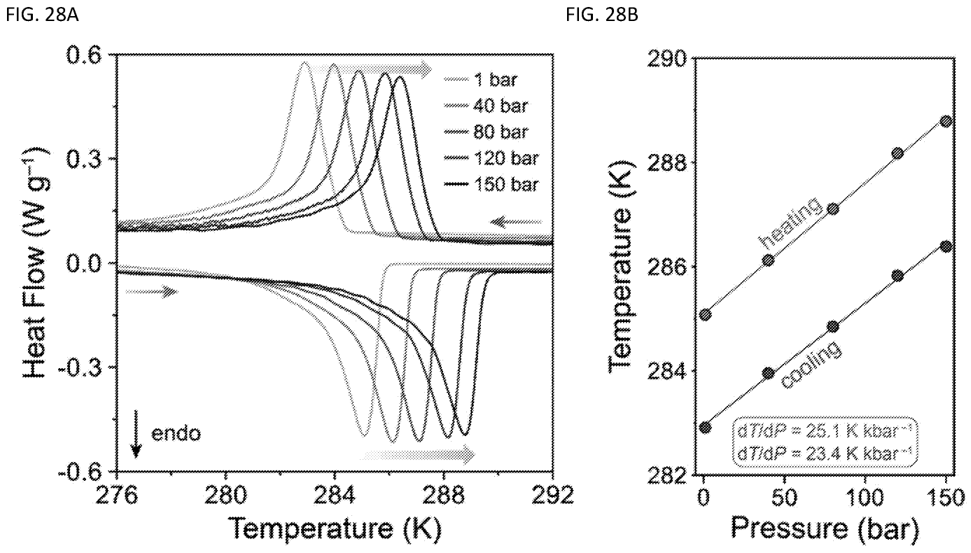

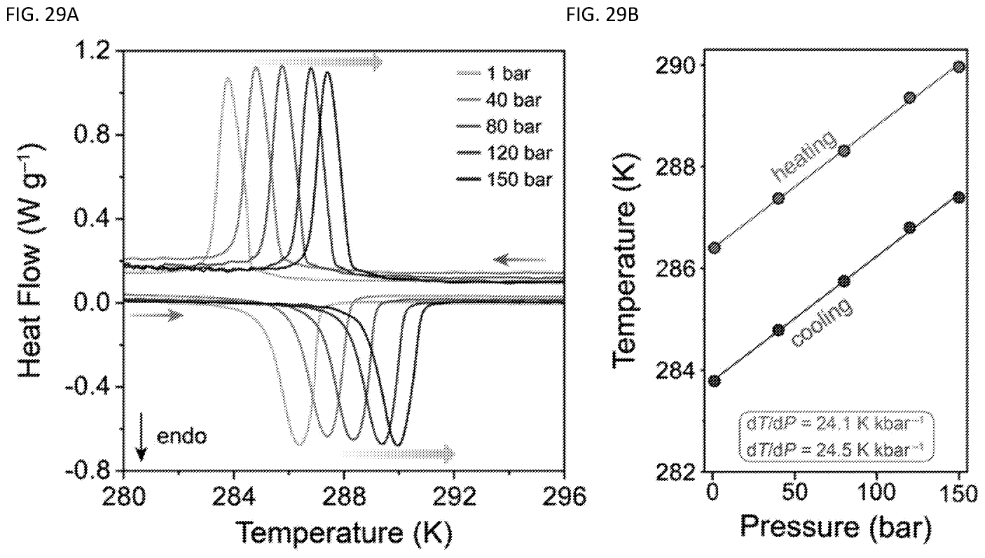

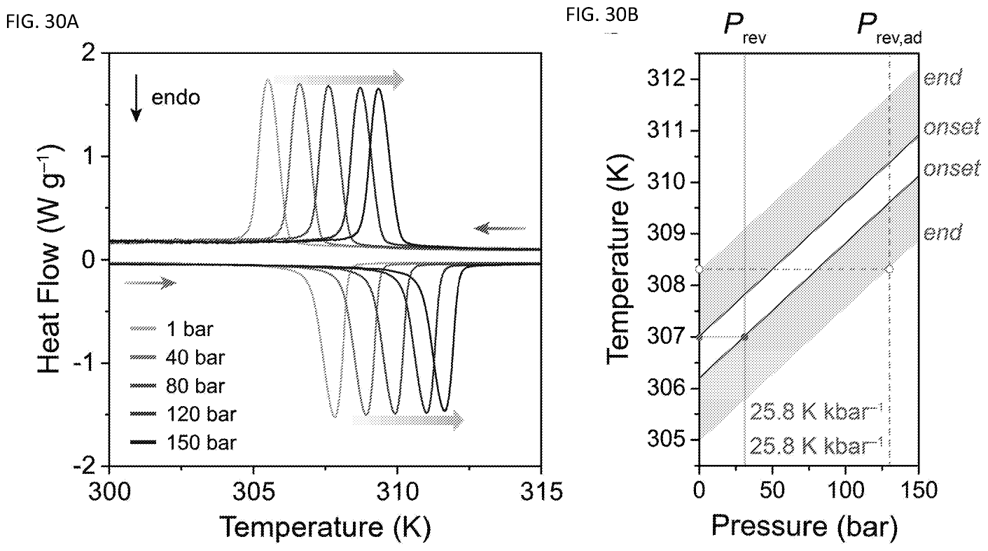

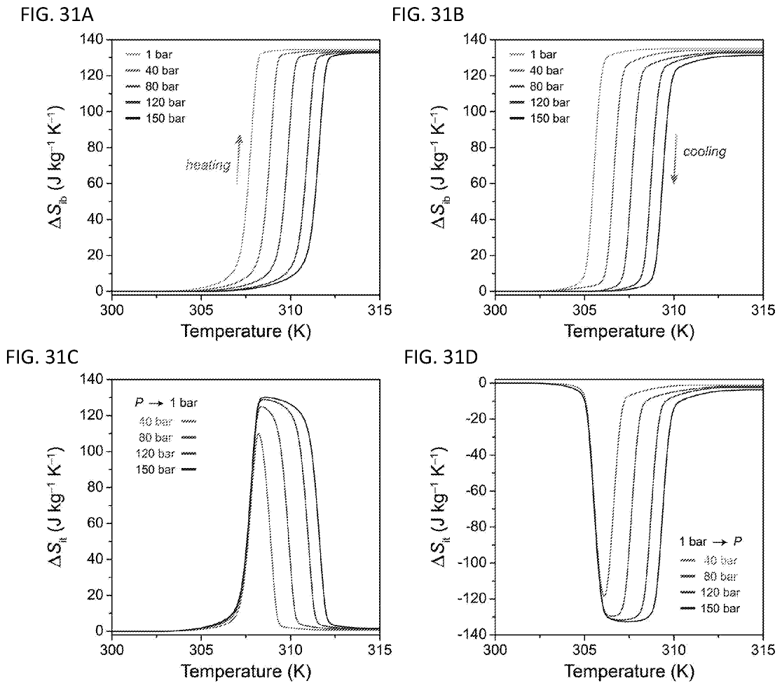

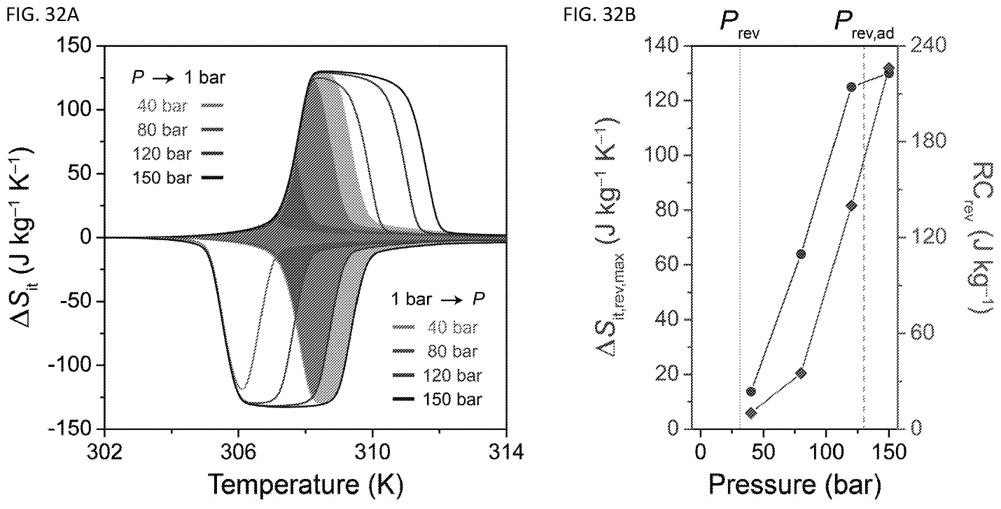

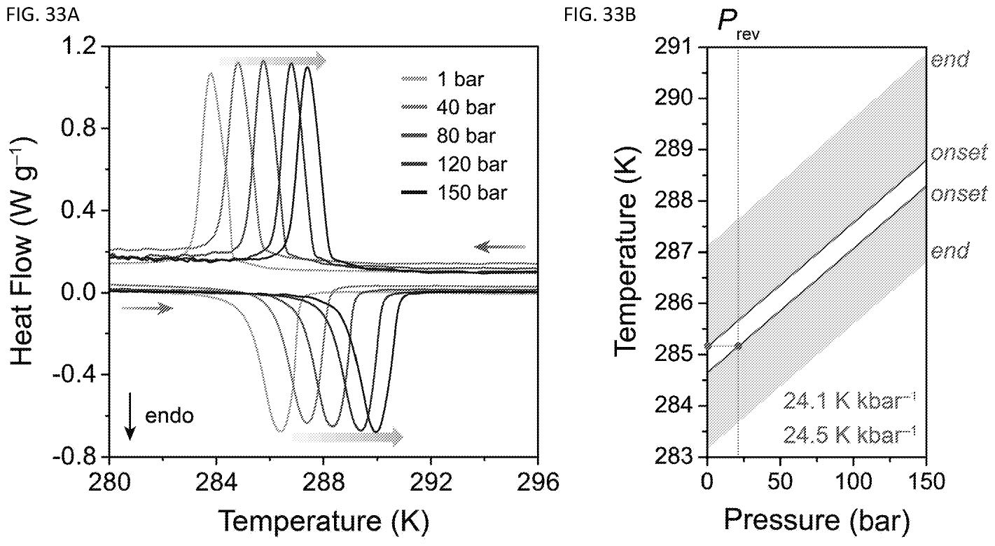

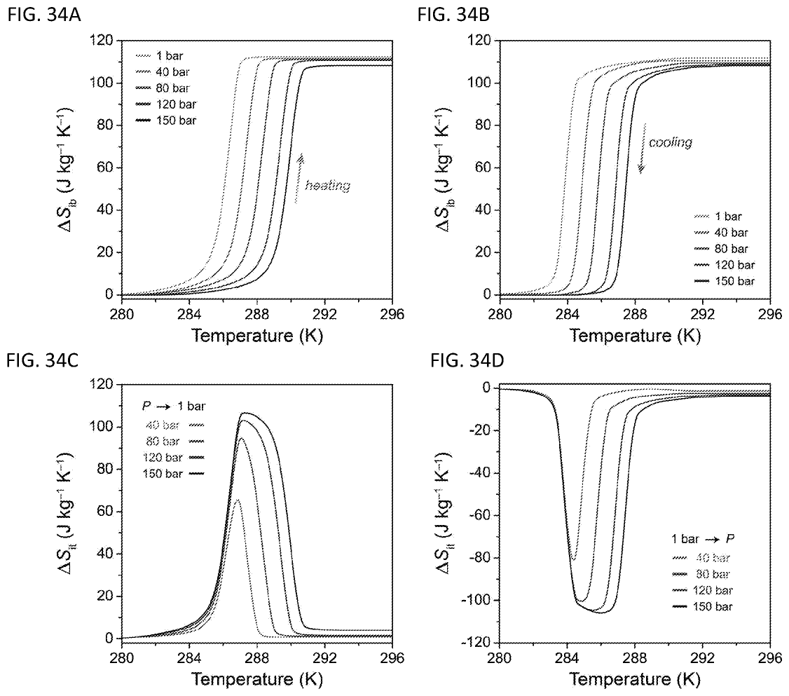

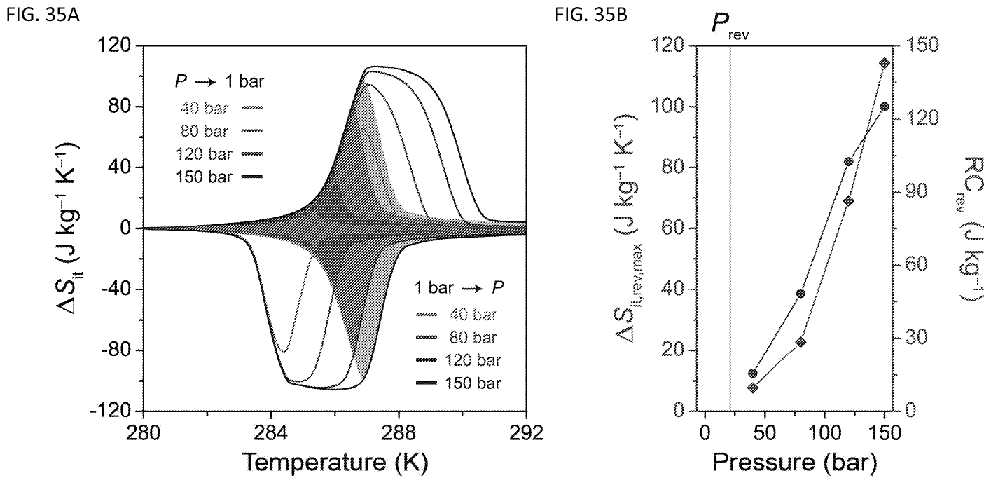

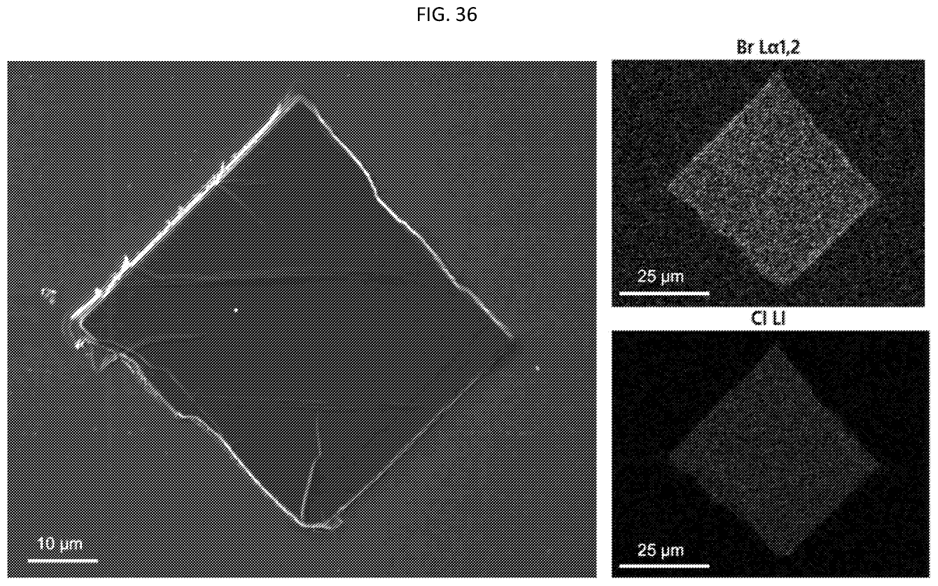

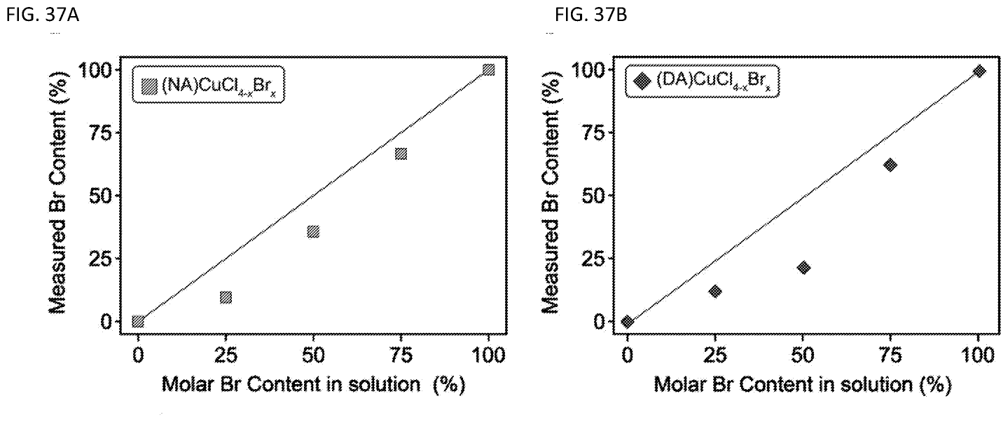

In addition, we have synthesized compositionally engineered mixed halide 2D metal-halide perovskites, e.g., replacing all—or a portion—of Cl anions with Br anions for mixed-halide systems, e.g., having formula [(R¹)ₓ(R²)₁₋ₓ]₂MX_{y}X′^{4-y}, where R¹ and R² are long chain alkylammonium species(e.g., CₙH₂ₙ₊₁NH₃⁺, where n>3, e.g., >4, e.g., NA or DA) and where X and X′ are different halides, e.g., selected from Cl, Br, or I, e.g., (R-NH₃)₂MCl_{4-y}Br_{y} (0<y≤4), e.g., where M is a transition metal (e.g., Ti, V, Cr, Mn, Fe, Co, Ni, Cu, Zn, Nb, Mo, Rh, Pd, Cd, Re, Pt, or Hg) and R is NA or DA. Mixed halide compounds may also be “double mixed”, e.g., containing two halides (e.g., Cl and Br) and two different alkylammonium species. The different alkylammonium species may be in non-integer ratios, relative to the metal center (e.g., [(NA)_{0.75}(DA)_{0.25}]₂CuCl₄, [(NA)_{0.5}(DA)_{0.5}]₂CuCl₄, or [(NA)_{0.25}(DA)_{0.75}]₂CuCl₄, [(NA)_{0.25}(UA)_{0.75}]₂CuCl₄, [(NA)_{0.5}(UA)_{0.5}]₂CuCl₄, or [(NA)_{0.5}(DA)_{0.5}]₂CuCl₂Br₂). In compounds of the formula [(R¹)ₓ(R²)₁₋ₓ]₂MX_{y}X′_{4-y}, ‘y’ may be 0-4 (e.g., 0, 1, 2, 3, or 4) and ‘x’ may be between 0-1, e.g., about 0.1, 0.15, 0.2, 0.25, 0.3, 0.35, 0.4, 0.45, 0.5, 0.55, 0.6, 0.65, 0.7, 0.75, 0.8, 0.85, 0.9, or 0.95. Thermal and structural data on the mixed 2D metal-halide perovskite materials in shown in Tables 21-25. Some mixed 2D metal-halide perovskite materials are compared to non-mixed 2D metal-halide perovskite materials in FIGS. 12A and 12B.

In addition, additional functionalized layered perovskites may be formed by (i) combining two different modifications in a single chain (e.g., C₃OC₄OH) (ii) fluorinating the chain, (iii) synthesizing layered halide double perovskites of the form (R-NH₃)₄MM′X₈ where M is a monovalent cation such as Na⁺ and M′ is a trivalent cation such as Fe³⁺, X=Cl^{—}, Br^{—}. In some embodiments, one or more Cl, Br, or I halides may be replaced by F. In addition, additional functionalized layered perovskites may be formed from non-halide anions, e.g., CN^{—}, HCOO^{—}, N₃^{—}, N(CN)₂^{—}, BF₄^{—}, BH₄^{—}, PF₆^{—}, SCN^{—}, OCN^{—}. Incorporation of the larger anions can increase the pocket size for cations (e.g., the alkylammonium species), thus relatively larger cations e.g., the largest cations of Table 1, or even dialkylammonium cations described herein.

In addition to synthetic tunability and chemical diversity, layered halide perovskites display a number of other properties that are advantageous for practical solid-state cooling. First, the “soft” nature and high solution processability of layered halide perovskites enables thin films to be easily fabricated. The high processability not only presents rich opportunities for the design of miniaturized cooling devices but may also allow the invention to take advantage of microscopic mechanisms for barocaloric effects across various length scales (from bulk powders to thin films to atomically thin layers) and materials forms (from single crystals to microcrystalline powders to thin films). Second, phase transitions in appropriately designed layered perovskites display small thermal hysteresis (<4 K). Thus, reversible barocaloric effects can be achieved at small operating pressure prev. Given that these layered perovskites display high barocaloric coefficients (>20 K kbar⁻¹), this suggests that colossal reversible barocaloric effects can be realized at easily accessible pressures (<100 bar) (FIG. 6).

For comparison, organic plastic crystals—another class of colossal barocaloric materials—require relatively high operating pressure of 500-1,000 bar for reversible barocaloric effects. These two features—high processability and low operating pressure—allow various device architectures with minimal design constraints.

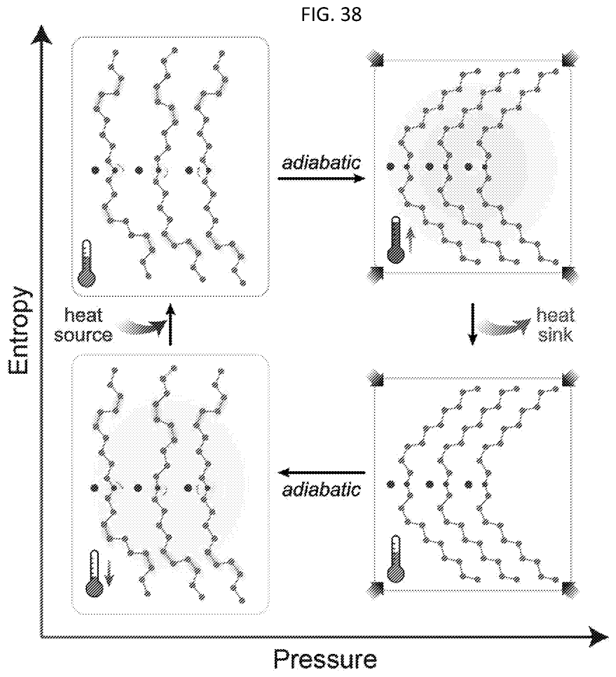

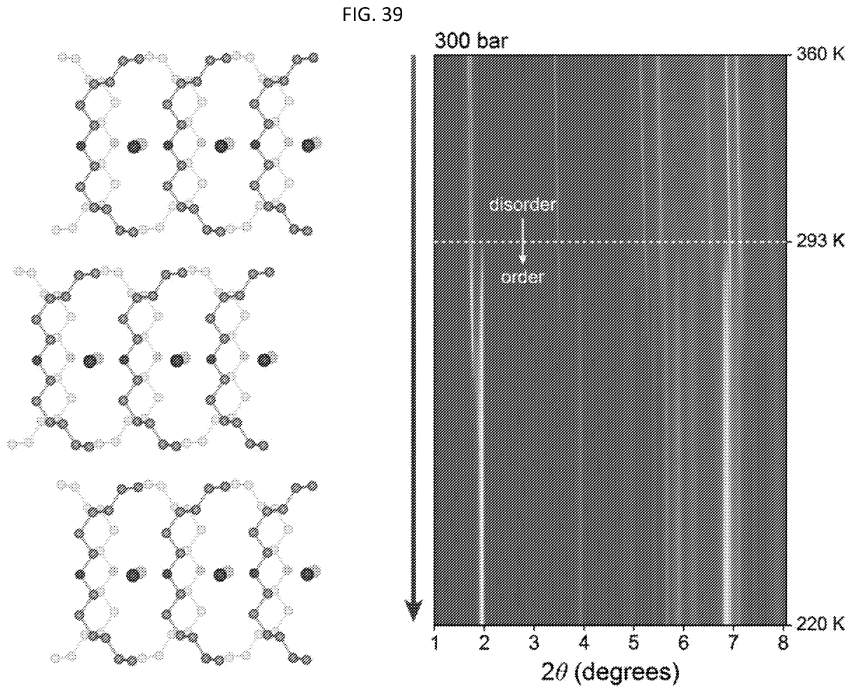

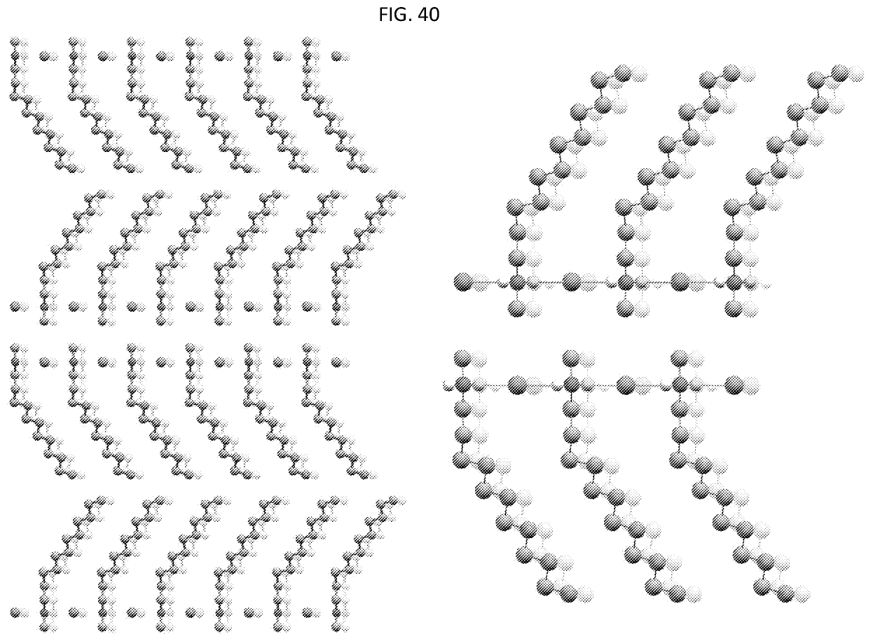

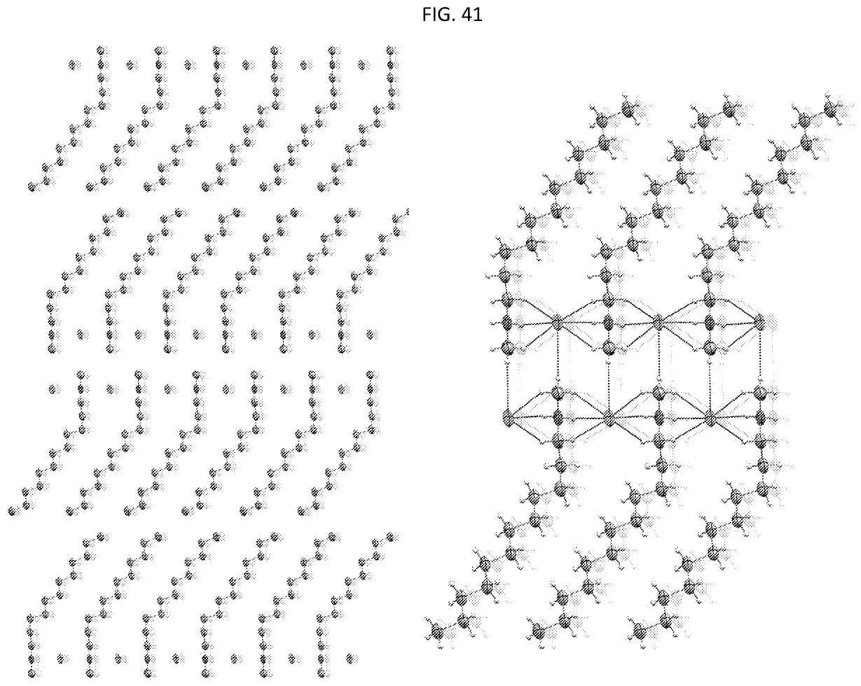

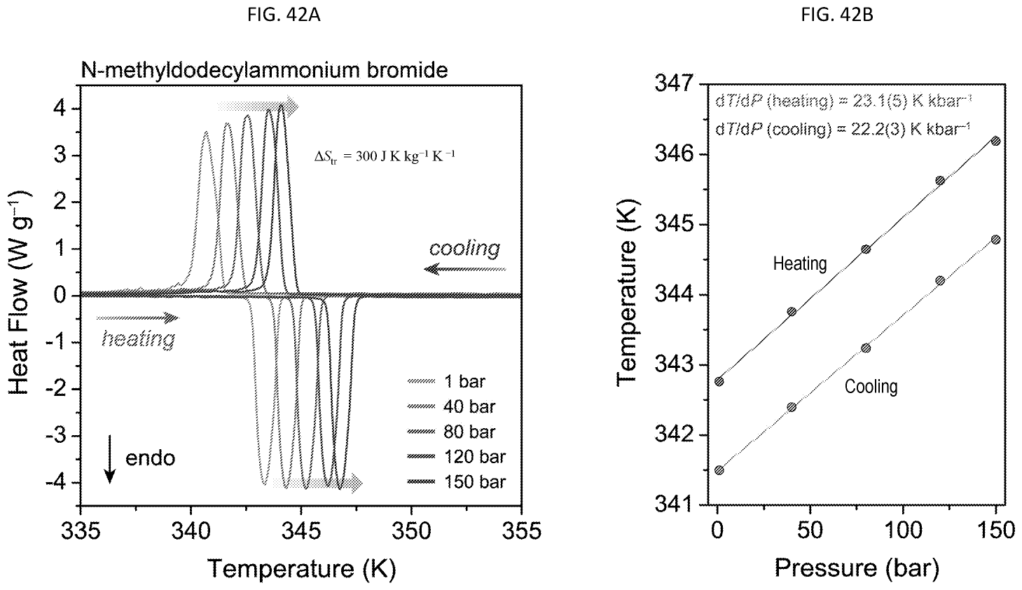

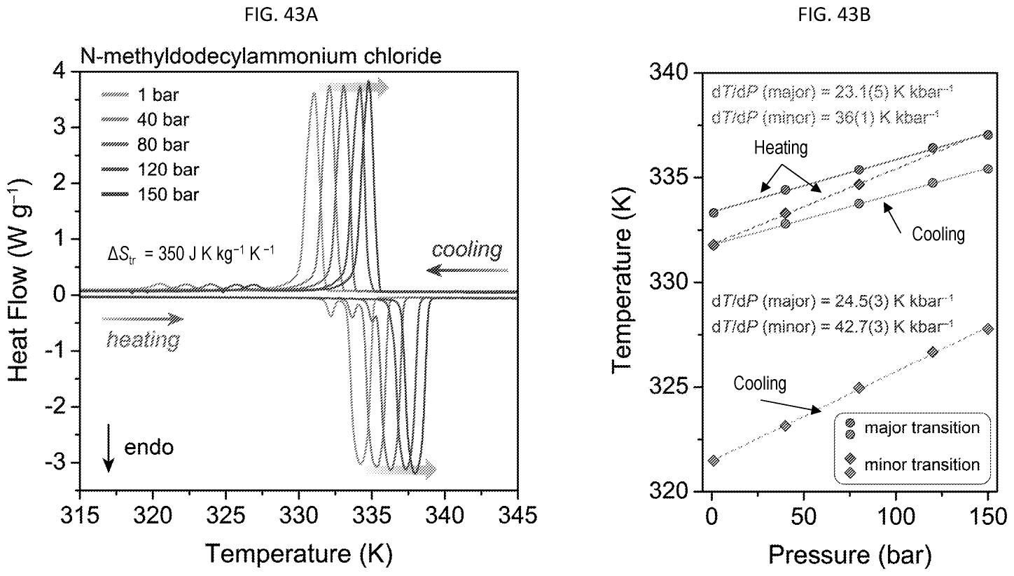

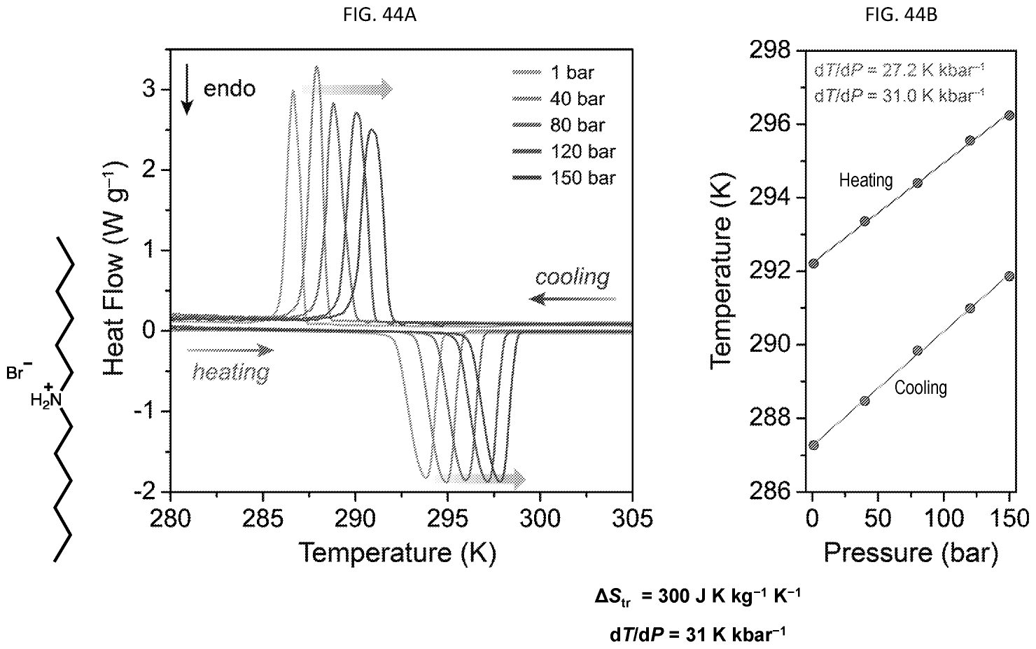

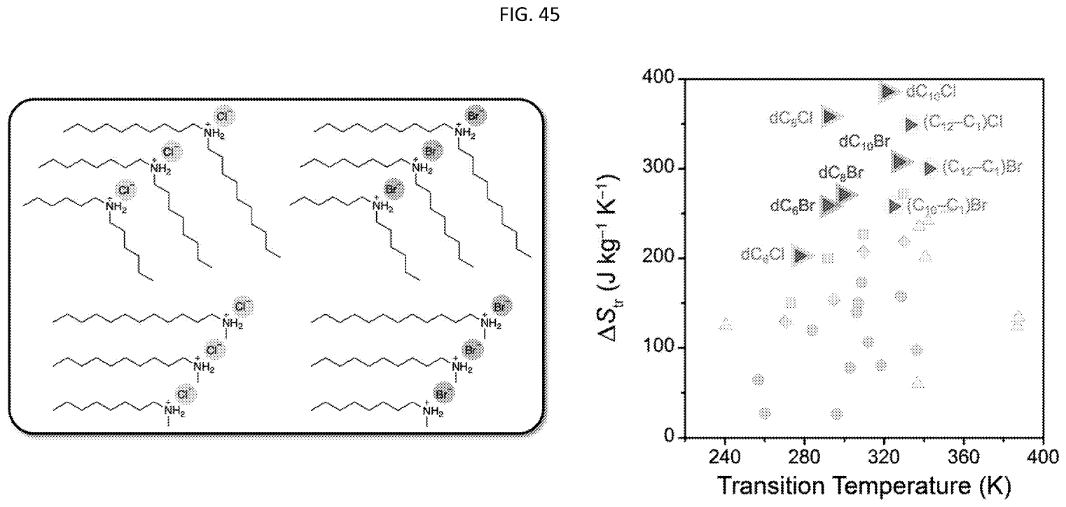

The confined chain melting described herein is a general mechanism to achieve colossal barocaloric effects because large latent heat, entropy change, and volume expansion—prerequisites to colossal barocaloric effects—can simultaneously emerge when long-chain molecules are forced to undergo large conformational changes in confined space. Note that the analysis and experiments described in this invention are readily applicable to other types of layered materials beyond hybrid perovskites. Any organic and inorganic materials that can be assembled into layers of organic material between layers of inorganic material are suitable to be used in the invention. Alternatively or in addition, organometallic materials including a layer-forming inorganic component and an organic component including long, optionally substituted alkyl chains may be used, for example, the following barocaloric materials: di-n-alkylammonium salts (e.g., compounds of formula (CₙH₂ₙ₊₁)₂NH₂X (n>3 (e.g., >4, e.g., C₄₋₃₆ alkyl chains) or compounds of formula (CₙH₂ₙ₊₁)(CₘH₂ₘ₊₁)NH₂X, where n is 1-3 or 4-36 and m is 4-36, e.g., where n=m, or where n=1-3 (e.g., 1) and m=4-36 (e.g., 6, 8, 10, or 12); where X is a monoanionic species, e.g., a halide (e.g., F, Cl, Br, or I) or a non-halide anion, such as NO₃—, ClO₃—, ClO₄—, H₂PO₄—, HSO₄—, CN^{—}, HCOO^{—}, N₃^{—}, N(CN)₂^{—}, BF₄^{—}, BH₄^{—}, PF₆^{—}, SCN^{—}, or OCN^{—}), see FIGS. 38-45 and Tables 26-29), alkylammonium-modified layered silicates, and layered metal-alkylphosphonate salts may also be used in systems, devices, and methods of the invention (see, e.g., Table 20). These compounds all undergo reversible, solid-solid, phase transition near room temperature with large entropy and volume change. Alkylammonium species may have odd or even numbered main chains. Alkylammonium species may have main chain lengths of greater than 36 carbons (e.g., up to 38, 40, 45, 50, 60, 75, or 100 carbons). Alkylammonium species of the invention may be quaternary ammonium species (e.g., Me₃N(CₙH₂ₙ₊₁)X or Me₂N(CH₂ₙ₊₁)₂X, where n is >3, e.g., >4, e.g., 4-36, and where X is a monoanionic species, e.g., a halide). Dialkylammonium species of the invention may be asymmetric, e.g., having formula (CₙH₂ₙ₊₁)(CₘH₂ₘ₊₁)NH₂X where n and m are both >3 (e.g., 4-36) but are not the same length.

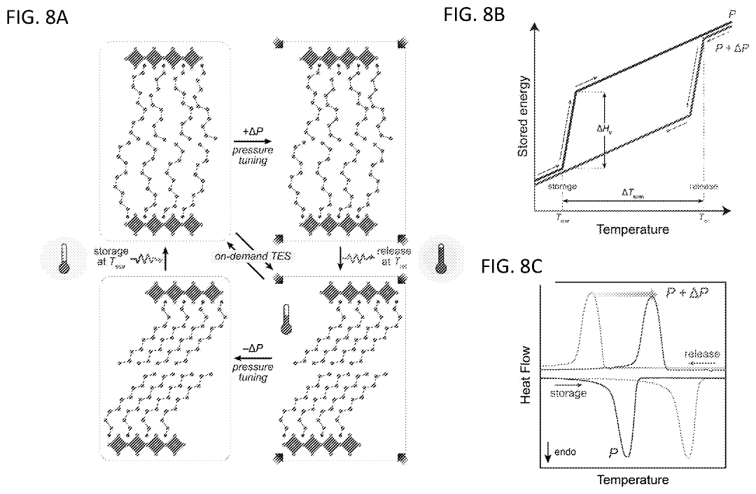

Chain-melting phase transitions and their pressure sensitivity (dTₜᵣ/dP), discussed here in the context of barocaloric cooling (using the 2-D perovskites as proof-of-concept examples), offer a new mechanism to realize highly efficient and tunable thermal energy storage, whereby application of hydrostatic pressure can be used to tune the storage temperature, Tₛₜₒᵣ, and release temperature, Tᵣₑₗ (FIGS. 8A and 8B). The difference between the barocaloric cooling and pressure-tunable thermal energy storage (PT-TES) lies in the specific operating cycle that is employed. During a typical PT-TES cycle—where thermal energy is stored and released by a change in temperature—the pressure change, ΔP, can occur outside of transition. Here, the pressure change does not drive phase transitions and is only used to tune the transition temperatures for thermal energy storage and release at different temperatures. Due to the simplicity of the operation, we anticipate that PT-TES can be readily implemented in various environments, with a low actuator-to-material volume ratio that will contribute to high volumetric working capacity. We also note that, once the materials are thermally charged, application or removal of the pressure, can directly induce phase transitions, leading the on-demand release of stored thermal energy. This process, despite its simplicity, does not require strict maintenance of isothermal conditions, as long as the pressure change is sufficiently high.

The material properties required for efficient PT-TES are similar to those required for efficient barocaloric cooling. Materials for PT-TES should undergo solid-state phase transitions with high thermal changes (ΔSₜᵣ), high sensitivity to pressure (dTₜᵣ/dP), and small hysteresis (ΔT_{hys}), because the temperature span of the PT-TES operation (ΔTₛₚₐₙ), defined here as the difference between Tᵣₑₗ and Tₛₜₒᵣ, is calculated by, ΔTₛₚₐₙ=(dTₜᵣ/dP)×ΔP−ΔT_{hys}. Note that PT-TES materials will benefit from having large ΔTₛₚₐₙ and small ΔP, and materials with high dTₜᵣ/dP and low ΔT_{hys}, as well as large ΔSₜᵣ and ΔHₜᵣ, will be suitable for this. Another difference is, unlike barocaloric cooling (which requires phase transition temperatures near or below ambient temperature), thermal energy storage materials are needed across a broad temperature range. In this context, compositions of the invention, e.g., the two-dimensional metal-halide perovskites—first highlighted here in the context of barocaloric cooling due to their large thermal changes (ΔSₜᵣ and ΔHₜᵣ), high pressure sensitivity (dTₜᵣ/dP), and small hysteresis (ΔT_{hys})—provide a versatile platform to achieve efficient the PT-TES in the broad temperature range, as their transition temperatures and sensitivity to pressure can be readily manipulated by changing the length of hydrocarbon chains to cover a wide temperature range (from 250 K to 400 K) (Tables 2 to 5). Layered compounds of the invention, e.g., with long-chain hydrocarbons, will be highly competitive PT-TES materials, due to their beneficial phase-change properties, synthetic tunability, and pressure dependence, similar to those of the 2-D perovskites (Table 20) exemplified herein.

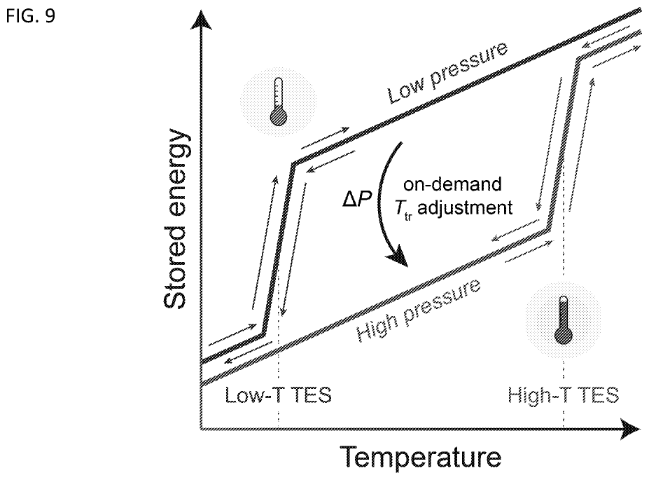

As shown in FIG. 9, we believe that this biggest impact of PT-TES approach is to adjust the phase-change temperature of the TES material on demand, such that the working temperature can be optimized for changing demands of the thermal energy storage and thermal management.

### Effect of Pressure Transmitting Medium

The invention also includes methods of enhancing barocaloric cycles based on the properties of the pressure-transmitting medium. The pressure transmitting medium (PTM) can affect the properties of the phase transitions of the barocaloric cycles in materials including long-chain hydrocarbons. The effects include inverse barocaloric effects for compounds (e.g., with long-chain hydrocarbons) that undergo reversible chain-melting transitions. Gaseous PTMs may induce changes in thermal properties of materials with long alkyl chains (e.g., those of the invention) by permeating into and interacting with the materials of the composition, e.g., by permeating into free volume in the organic layer. Gases that can permeate into the composition are preferably inert gases that can also interact with the composition at the microscopic level (e.g., non-covalently interact, e.g., via Van der Waal's-type interactions, e.g., via dispersion forces). Both the extent of permeation (e.g., amount of gas molecules in the free volume/interacting with the composition) and degree and nature of interaction (e.g., strength of interaction, e.g., determined by a molecule or atom's size, shape, polarizability, etc.) can determine the effect of the PTM on thermal transitions of the composition. Exemplary PTM gases include nitrogen, argon, krypton, xenon, methane, ethane, propane, butane, sulfur hexafluoride, or carbon dioxide. Gases that permeate and interact sufficiently with the composition (e.g., argon into (NA)₂CuBr₄)) can cause the barocaloric effect of the composition to be inverted.

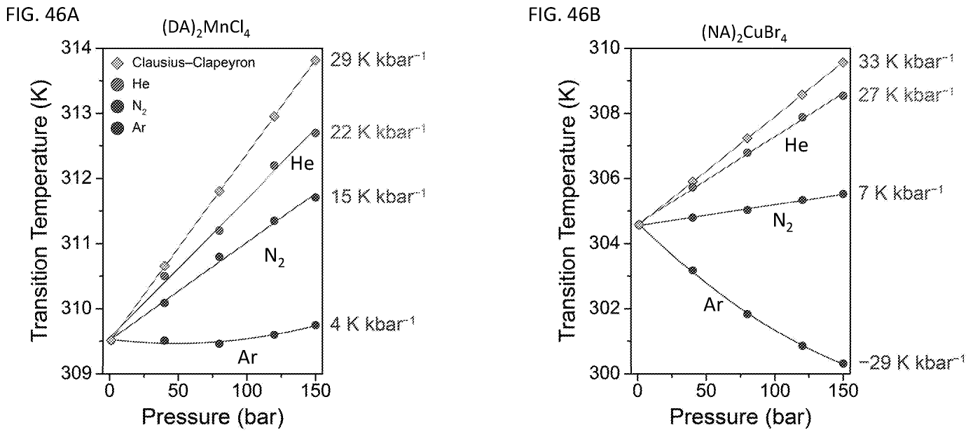

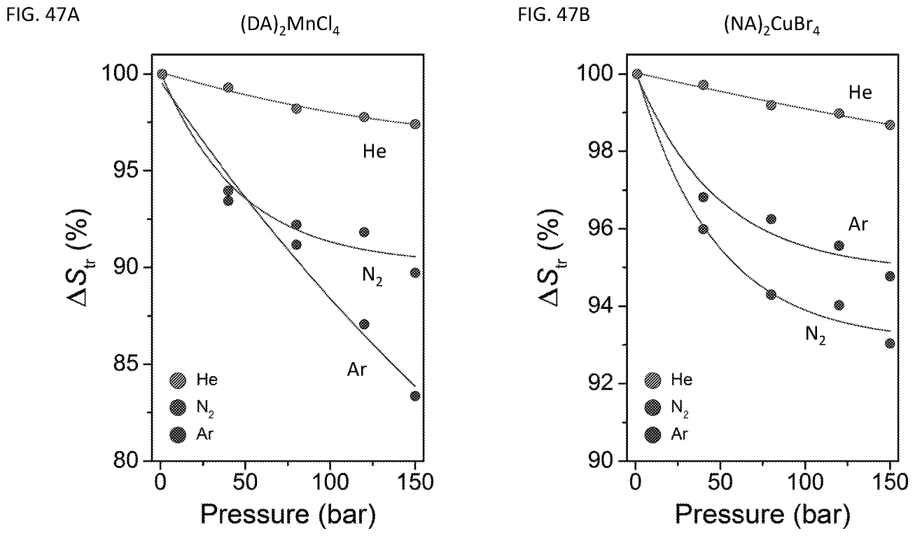

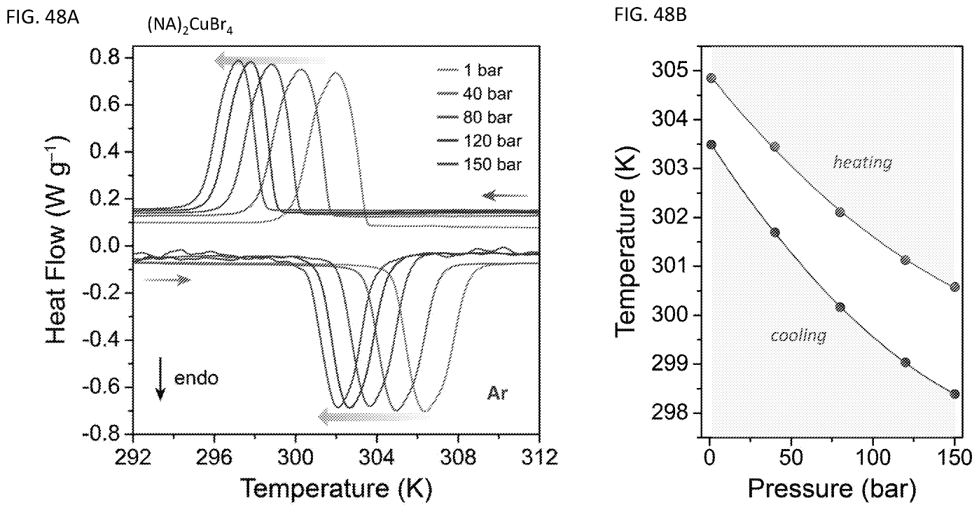

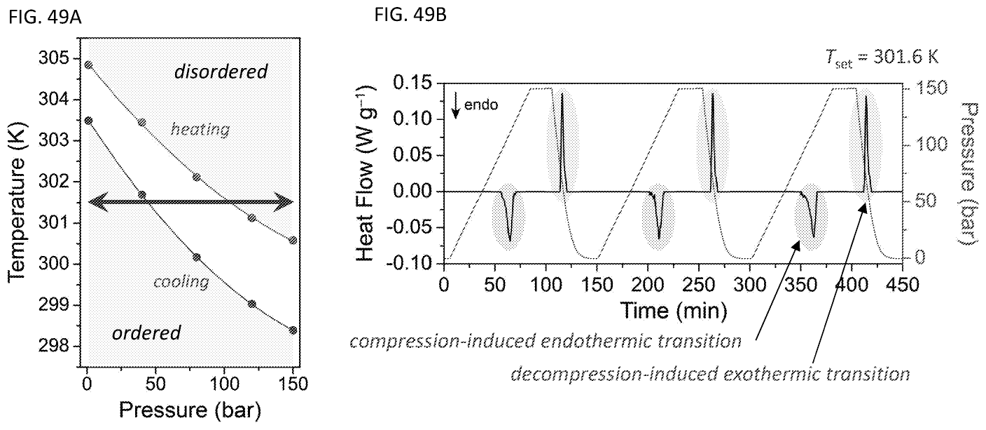

In our recent studies, we discovered that the pressure sensitivity (dT/dP) of chain-melting transitions in representative 2-D perovskites—(DA)₂MnCl₄ and (NA)₂CuBr₄—depends on the identity of pressure-transmitting medium (PTM). Specifically, as shown in FIGS. 46A and 46B, high-pressure differential scanning calorimetry (HP-DSC) experiments revealed that the use of polarizable gas (such as Ar) lowers the dT/dP values, from 22 K/kbar under He to 4 K/kbar under Ar for (DA)₂MnCl₄; from 27 K/kbar under He to −29 K/kbar under Ar for (NA)₂CuBr₄. We also note that the pressure-transmitting medium appears to influence only transition temperatures with minimal impact on transition entropy (FIGS. 47A and 47B). Most notably, under Ar environments, (NA)₂CuBr₄ displays inverse barocaloric effects, where the increase in pressure leads to a decrease in transition temperature (i.e., dT/dP<0), as indicated by heat flow traces and phase diagram obtained from Ar HP-DSC experiments (FIGS. 48A and 48B). To directly evaluate the magnitudes of the PTM-driven inverse barocaloric effects, we used quasi-isothermal HP-DSC experiments, where phase transitions are induced by pressure changes under isothermal conditions (see FIGS. 49A and 49B). This experiment demonstrated that the compression induces an endothermic transition from ordered (low-temperature) phase to disordered (high-temperature) phase, whereas the decompression induces an exothermic transition from disordered phase to ordered phase. Note that the sample temperature was held at 301.6 K because the (P, T) phase diagram shown in FIG. 49A indicates that the pressure swing of 150 bar is sufficient to induce full phase change at that set temperature. Although these phenomena were unexpected, and the microscopic origin of these phenomena remains to be investigated, we hypothesize that the inverse barocaloric effects in (NA)₂CuBr₄ originates from its large free volume that enables facile absorption of (or interactions with) the polarizable Ar gas molecules in disordered organic bilayers.

These results fully confirm the validity of this phenomenon and establish that (NA)₂CuBr₄—and other related barocaloric compounds (e.g., with long-chain hydrocarbons), e.g., those described herein, more broadly—displays highly reversible, giant inverse barocaloric effects under a pressure-transmitting medium that can interact with hydrocarbon chains (and the lattice).

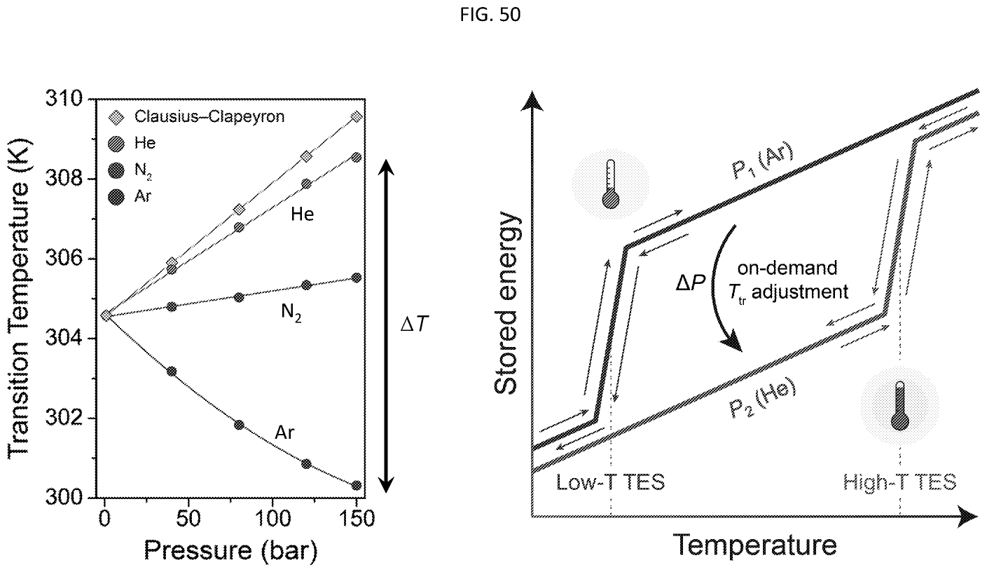

Most inverse barocaloric effects arise from a decrease in volume upon increase in a degree of freedom (during thermally-induced phase transitions). The mechanism described herein is unique for the following reasons. First, the material (i.e., the host lattice, e.g., a barocaloric material of the invention) still undergoes a volume expansion upon the thermally-induced order-to-disorder transition, and the inverse barocaloric effect is entirely driven by the permeation and absorption of the PTM into the lattice. Second, the magnitude of inverse barocaloric effects can be tuned via judicious selection of pressure medium. As shown in FIGS. 46A and 46B, the use of N₂ as PTM lowers the pressure sensitivity (dT/dP); but still the dT/dP is positive (i.e., normal barocaloric effects). We anticipate that the mixing of two different PTM (e.g., via gas exchange mechanisms) will allow us to continuously access a wide range of pressure sensitivity (from ˜33 K/kbar with non-interacting/non-permeating PTM to <−29 K/kbar with interacting/permeating PTM). Third, to the best of our knowledge, the phenomenon shown here is the first case, where a single material reversibly displays both normal and inverse barocaloric effects. Methods and systems of the invention can make use of these tunable and “on-demand” inverse barocaloric effects, which we expect will play a very important role in realizing practical barocaloric cooling devices, methods and systems. For instance, in response to compression, a normal barocaloric material will increase its temperature, whereas an inverse barocaloric material will decrease its temperature. By utilizing these two materials in series, it may be possible to increase the temperature span and facilitate heat transfer processes for barocaloric cooling cycle. Additionally, as described in FIG. 50, we expect that this PTM effect and inverse barocaloric effects can provide a new mechanism for tunable thermal energy storage (TES).

### Methods

Methods of the invention may include providing heat energy (e.g., from a room, an AC system, heat transfer medium, heat pump, heat sink, etc.) to a composition of the invention (e.g., a 2D perovskite). The heat energy may cause alkyl chains in the composition to undergo a phase transition (e.g., from an ordered to a disordered state, e.g., in a thermal energy storage system) or there may be no phase transition until pressure is applied (e.g., in a barocaloric cooling system). Methods may be for refrigeration or heating.

In a barocaloric cooling system or method, providing compression to the composition releases latent heat in the composition, which may be removed, e.g., via a heat sink, e.g., a high surface area, high conductivity medium in thermal contact with the composition which may be itself cooled by, e.g., a fan. Removal of the heat is performed while the composition is still compressed, and removal of the compression allows the composition to return to a disordered state, cooling the composition as the endothermic transition occurs. At this point the cycle may be repeated with input of new heat energy.

In a barocaloric thermal energy storage system heat energy is provided to a composition of the invention causing it to undergo a phase transition to a disordered state. The disordered state is then modified by the application of compression in order to change the temperature at which heat is released.

Methods of the invention may also include selecting or otherwise controlling the pressure transmitting medium to modulate the barocaloric cycle. For example, selecting a gas (e.g., a high polarizability gas) that sufficiently permeates and interacts with the composition at the microscopic level as the PTM to change the temperature of phase transitions in the barocaloric material, or to induce inverse barocaloric effects such as described herein. Methods may include modulating the barocaloric cycle by altering a ratio of polarizable and on-polarizable gases used as a mixed in a PTM. Methods may include selecting a gas as the PTM that does not interact, or minimally interacts, with the composition (e.g., He), e.g., to not induce changes in thermal properties, or to revert changes caused by an interacting gas.

### Systems and Additional Components

Systems of the invention may include components to provide compressive force to the composition, e.g., pumps, pistons, actuators (e.g., mechanical, hydraulic, or pneumatic, etc., actuators), presses (e.g., mechanical, hydraulic, or pneumatic, etc., presses), piezoelectric actuators, levers, etc. Systems may also include components to transfer or remove heat energy, e.g., pumps, heat sinks, thermoelectrics, fans, chiller pumps, etc. A system of the invention may also include a power source, e.g., to power the source of compressive force, the cooling or heat transfer components, etc. Systems of the invention may include a pressure transmitting medium (PTM), e.g., a gas. The PTM may be a non- or minimally-interacting gas (e.g., a low polarizability gas, e.g., He) or a polarizable gas (e.g., N₂, Ar, Kr, Xe, methane, ethane, propane, butane, sulfur hexafluoride, or carbon dioxide). Systems of the invention may include a pump for controlling a pressure transmitting medium (e.g., a mixture of gases), such as pumps, gas reservoirs (e.g., tanks, cylinders, etc.), pressure sensors, actuators, valves, etc. The PTM may not be a gas, for example, the PTM may be an oil, e.g., a fluorocarbon oil, silicone oil, etc.).

### EXAMPLES

The 2-D perovskite (DA)₂MnCl₄ (DA=decylammonium) was selected as a barocaloric material because of its large phase-transition entropy (ΔSₜᵣ=230 J kg⁻¹ K⁻¹) and enthalpy (ΔHₜᵣ=71 kJ kg⁻¹), near-ambient phase transition temperature (Tₜᵣ=310 K), and lightweight, nontoxic elemental composition. At ambient temperature and pressure, (DA)₂MnCl₄ adopts an ordered monoclinic structure (low-temperature, LT, phase) with bilayers of hydrocarbon chains—each of which contain a single gauche C—C bond (C2-C3) and seven trans C—C bonds—aligned parallel to one another and tilted 48.3(1)° with respect to the Mn—Cl plane (FIG. 5E, Table 10). Upon heating above 310 K, the compound undergoes a first-order phase transition to an expanded orthorhombic lattice (high-temperature, HT, phase) with dynamically disordered hydrocarbon chains that have liquid-like conformational degrees of freedom. The large increase in entropy during the transition is due to both flipping of the alkylammonium cations between two favorable orientations within the Mn—Cl pockets and internal rotations of C—C bonds that create dynamically disordered conformational defects within the hydrocarbon chains. Note that 90% of the overall molar entropy change results from conformational disorder, with approximately six C—C bonds freely converting between three rotameric states—gauche⁺, gauche⁻, and trans—in the HT phase.

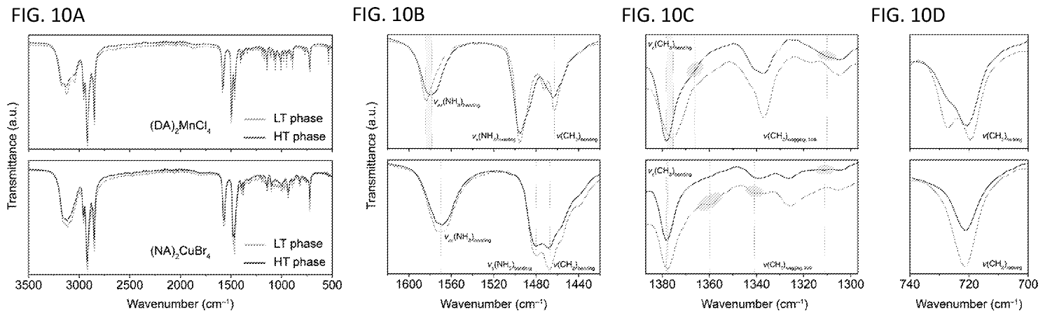

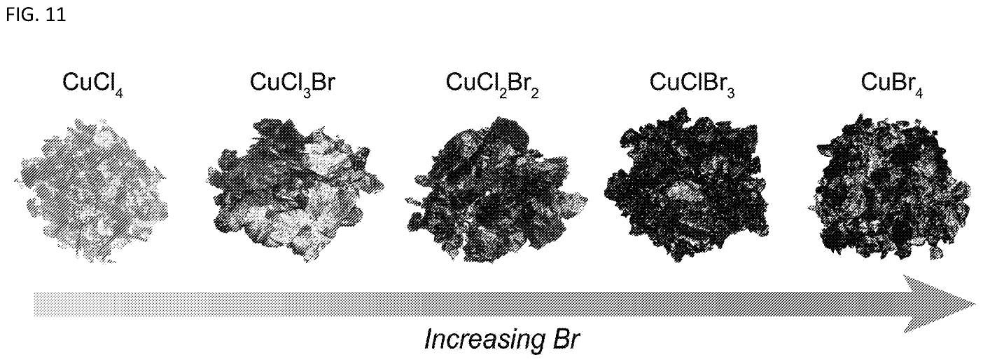

Differential scanning calorimetry (DSC) measurements (FIGS. 5A-5C) at ambient pressure show that the hydrocarbon chain-melting transition is sharp and fully reversible with a thermal hysteresis, ΔT_{hys}, of just 1.4 K at a scan rate of 2 K min⁻¹ (FIG. 5A). Variable temperature single crystal X-ray diffraction structures (e.g., FIGS. 5E-5H) at ambient pressure show that the phase transition is accompanied by an increase in interlayer distance of 2.115(2) Å as the alkylammonium cations tilt further away from the Mn—Cl plane to create additional space between disordered hydrocarbon chains in the HT phase (FIG. 5F, Table 10). This uniaxial expansion leads to an 8.0% increase in the volume of the compound (ΔVₜᵣ=65.1 cm³ kg⁻¹) during the phase transition (FIG. 10C). Based on the measured volume and entropy changes, the Clausius-Clapeyron equation, dTₜᵣ/dP=ΔVₜᵣ/ΔSₜᵣ, can be used to predict a barocaloric coefficient for (DA)₂MnCl₄ of 28.3 K kbar⁻¹, which would represent one of the highest values reported for a barocaloric material with a ΔSₜᵣ above 20 J kg⁻¹ K⁻¹ (Table 18).

To directly evaluate the pressure dependence of the phase transition temperature, isobaric DSC experiments were performed under applied hydrostatic pressures of up to 150 bar using He as the pressure-transmitting medium. The phase transition shifts to higher temperatures as the pressure is increased, with a measured dTₜᵣ/dP of 22.1±0.7 K kbar⁻¹ during heating and 20.6±0.8 K kbar⁻¹ during cooling (FIG. 6A). Importantly, the application of pressure does not lead to any significant changes to the phase transition width. Moreover, ΔSₜᵣ remains within 97% of its ambient pressure value at 150 bar and does not decrease upon repeated thermal cycling at 150 bar. Variable temperature and pressure powder X-ray diffraction (PXRD) experiments confirm that structural phase transitions with similar volume changes still occur at pressures up to at least 360 bar (FIG. 6G), and the dTₜᵣ/dP of 18.6±1.0 K kbar⁻¹ during cooling over this extended pressure range is in close agreement with that measured by HP-DSC (FIG. 6H). However, the dTₜᵣ/dP values determined by PXRD and HP-DSC are lower than those predicted using the Clausius-Clapeyron equation.

To investigate the origin of the different experimental and predicted barocaloric coefficients, we used helium pycnometry to directly measure the volume change of (DA)₂MnCl₄ during the phase transition (FIG. 5C). In particular, we expected that He might permeate into the disordered organic bilayer of the HT phase—owing to its increased free volume—while being excluded from the denser, crystalline organic bilayer of the LT phase. This would lead to a lower effective volume change during the phase transition since He permeation would reduce the amount of additional volume that was occupied by the expanded phase. Indeed, the volume change measured by pycnometry is 16 cm³ kg⁻¹ lower than that determined by crystallography, and this lower effective volume change yields a predicted dTₜᵣ/d P of 21.4±1.5 K kbar⁻¹ that matches the HP-DSC and PXRD values (Table 9). Although effects of the pressure-transmitting medium are not typically considered when evaluating barocaloric materials, this result provides a pathway to realizing a higher dTₜᵣ/dP by preventing the pressure-transmitting medium from entering the disordered phase through, for instance, encapsulation, the use of a larger fluid, or the application of mechanical pressure. Regardless, the dTₜᵣ/dP that can be achieved using He to transmit hydrostatic pressure is higher than many barocaloric materials, which, along with the large ΔSₜᵣ and small hysteresis, presents considerable advantages for barocaloric cooling.

Under the cyclic operating conditions of a barocaloric cooling system, the lowest possible operating pressure is set by the pressure that must be applied to induce a reversible entropy change, Pᵣₑᵥ, when cycling to and from ambient pressure. For a conventional barocaloric effect, Pᵣₑᵥ corresponds to the pressure at which the onset temperature of the exothermic phase transition is equal to the onset temperature of the endothermic phase transition at 1 bar. As such, Pᵣₑᵥ is proportional to the thermal hysteresis at 1 bar and inversely proportional to the barocaloric coefficient for the exothermic transition, with Pᵣₑᵥ=ΔT_{hys}/(dTₜᵣ/dP)_{cooling} (16). Owing to its low ΔT_{hys} and high dTₜᵣ/dP, (DA)₂MnCl₄ has a predicted Pᵣₑᵥ of just 66 bar.

This low Pᵣₑᵥ was further confirmed by calculating the isothermal entropy changes (ΔSᵢₜ). To do so, we first obtained isobaric entropy changes (ΔS_{ib}) associated with the chain-melting transition as a function of temperature and pressure by integrating the HP-DSC heat flow signal, Q, obtained at a scan rate of \dot{T} over the temperature range from Tᵢ to T_{f}:

$$\\ Δ {S}_{ib} ( P , T ) = {{\int }^{{T}_{f}}}_{{T}_{i}} \frac{1}{T} \frac{Q ( P , T )}{\dot{T}} dT$$

ΔSᵢₜ curves were then calculated as the difference between ΔS_{ib} at ambient pressure and ΔS_{ib} at elevated pressure, with ΔS_{ib} values obtained from heating data corresponding to the disordering transition induced by a decrease in pressure (ΔSᵢₜ>0) and ΔS_{ib} values from cooling data corresponding to the ordering transition induced by an increase in pressure (ΔSᵢₜ<0). Excitingly, these ΔSᵢₜ curves show that a non-zero reversible entropy change can be induced below 80 bar, and the full entropy of the phase transition can be induced irreversibly by applying a pressure of just 100 bar (FIG. 6B). Moreover, a reversible entropy change of 75 J kg⁻¹ K⁻¹ can be accessed at a driving pressure of 150 bar, and the full entropy of the chain-melting transition would become reversible at only 270 bar. To the best of our knowledge, inducing a reversible entropy change of more than 200 J kg⁻¹ K⁻¹ through a pressure change of less than 300 bar is unprecedented in barocaloric materials. By assuming an average specific heat capacity, cₚ, of 1550 J kg⁻¹ K⁻¹ for the LT and HT phases that does not vary substantially with pressure, the equation ΔT_{ad, max}=TΔSᵢₜ/Cₚ can be used to estimate a maximum adiabatic temperature change of 42 K, which ranks among the highest values yet reported for barocaloric materials (Table 18).

Although quasi-direct methods of calculating isothermal changes-as well as adiabatic ones-from isobaric experiments are commonly used to evaluate barocaloric materials due to the challenge of maintaining isothermality—or adiabaticity—during direct variable pressure measurements, we performed quasi-isothermal HP-DSC experiments to more directly evaluate Pᵣₑᵥ by measuring pressure, rather than thermal, hysteresis. Specifically, we measured heat flow signals while cycling the pressure between 1 bar and 150 bar at 311 K. Note that isothermality is maintained until the phase transition onset pressure, which allows us to accurately determine pressure hysteresis. By comparing the onset pressures for compression-induced exotherms and decompression-induced endotherms, we were able to directly measure a pressure hysteresis for (DA)₂MnCl₄ of 70 bar, which is in excellent agreement with the predicted value of 73 bar at 311 K (FIG. 5C). To the best of our knowledge, the Pᵣₑᵥ value of (DA)₂MnCl₄ is the lowest reported for a barocaloric material with ΔSₜᵣ>45 J kg⁻¹ K⁻¹ (FIG. 7B). Note that three-dimensional metal-dicyanamide perovskites, [(C₃H₇)₄N][M(dca)₃] (M=Mn and Cd) display low Pᵣₑᵥ of 40 bar, due to their high dTₜᵣ/dP (23 K kbar⁻¹ and 38 K kbar⁻¹ for Mn and Cd compounds, respectively) and low hysteresis (1 K) (Table 18). These compounds, however, have low ΔSₜᵣ and undergo transitions well above room temperature.

In an effort to target barocaloric materials with large reversible entropy changes at even lower pressures, we searched for a 2-D perovskite that undergoes a chain-melting transition with a thermal hysteresis of less than 1 K. Unlike (DA)₂MnCl₄, however, the total entropy of the chain-melting transition in most 2-D perovskites is partitioned across one or more minor—lower entropy—phase transitions in addition to the principal transition (FIG. 3B, Tables 2 to 4). The presence of multiple successive transitions at different temperatures, although not necessarily detrimental to barocaloric cooling performance, complicates the evaluation of barocaloric properties because each minor and major transition has an independent hysteresis loop with a different pressure dependence. With a lack of suitable existing compounds, we synthesized a new 2-D perovskite that features a sharp chain-melting transition near ambient temperature with ultralow hysteresis and a high sensitivity to pressure. In particular, we expected that altering the inorganic-organic interface of 2-D perovskites through the substitution of different metal cations and halide anions might provide a pathway to reducing the hysteresis associated with the confined phase transition and increasing its sensitivity to pressure. More specifically, we hypothesized that larger Br anions would increase the distance between ammonium headgroups of hydrocarbon chains to provide more free volume, reducing the activation energy barrier for nucleation of an ordered bilayer phase during cooling or compression and increasing the sensitivity of the phase transition temperature to pressure.

Since Br anions can be more readily accommodated within Cu, rather than Mn, 2-D perovskites, we targeted a (CₙH₂ₙ₊₁NH₃)₂CuBr₄ compound with alkylammonium cations of appropriate length to place the chain-melting temperature near ambient temperature. We found that the new 2-D perovskite (NA)₂CuBr₄ (NA=nonylammonium) undergoes a chain melting transition at 303 K with a high ΔSₜᵣ (78 J kg⁻¹ K⁻¹), hysteresis of only 0.4 K, and no minor phase transitions within at least 60 K of the principal transition (FIG. 5B). Variable-temperature PXRD experiments reveal that the phase transition involves a 4.0% increase in volume (ΔVₜᵣ=24.9 cm³ kg⁻¹), and crystal structures of the LT and HT phases show that the phase transition leads to an increase in interlayer distance of 1.630(2) Å, which is consistent with conformational disordering of the chains (FIG. 5H, Table 11). The crystallographic volume change yields a predicted barocaloric coefficient of 32 K kbar⁻¹ using the Clausius-Clapeyron equation, while the volume change determined by He pycnometry (ΔVₜᵣ=20.0 cm³ kg⁻¹)—which accounts for He permeation into the HT phase—yields a predicted barocaloric coefficient of 25.6 K kbar⁻¹ (FIG. 5D, Table 9).

Isobaric HP-DSC experiments confirmed that (NA)₂CuBr₄ features a high barocaloric coefficient with dTₜᵣ/dP of 26.9±0.4 K kbar⁻¹ and 26.5±0.5 K kbar⁻¹ during heating and cooling, respectively (FIG. 6D). In fact, these values represent one of the highest sets of barocaloric coefficients ever measured among barocaloric materials with ΔSₜᵣ>20 J K⁻¹ kg⁻¹. In addition, variable pressure PXRD experiments show that the structural transition persists to at least 300 bar with a similar volume change and average barocaloric coefficient (25.9±1.0 K kbar⁻¹) (FIGS. 61 and 6J). As a result of its high barocaloric coefficient and ultralow hysteresis, (NA)₂CuBr₄ has a record-low Pᵣₑᵥ of 16 bar, which is within the pressure range already accessed during commercial vapor-compression refrigeration cycles (Table 18). The low value of Pᵣₑᵥ was confirmed through quasi-isothermal pressure cycling experiments at 307 K, where we directly measured a pressure hysteresis of 25 bar (FIG. 6F). Moreover, a reversible entropy change of 68 J kg⁻¹ K⁻¹ (90% of ΔSₜᵣ at 1 bar) can be accessed at a driving pressure of just 150 bar (FIG. 6E). Based on an average heat capacity of 800 J kg⁻¹ K⁻¹, (NA)₂CuBr₄ is predicted to have a maximum adiabatic temperature change of 21 K.

To provide additional insight into the structural and chemical factors that influence barocaloric effects in 2-D perovskites, we used X-ray crystallography and IR spectroscopy to compare the nature of the chain-melting transition in (NA)₂CuBr₄ and (DA)₂MnCl₄. In particular, we hypothesized that the increased size of the halide pocket in (NA)₂CuBr₄ (30.5 Å²) relative to (DA)₂MnCl₄ (26.3 Å²)—coupled with weaker N—H . . . Br hydrogen bonds at the organic-inorganic interface—would lead to more conformational disorder in the LT phase of the Cu compound. This would reduce the entropy difference between the LT and HT phases and explain the 56% lower molar entropy change of (NA)₂CuBr₄, as well as the lower entropy changes generally observed across longer-chain (CₙH₂ₙ₊₁NH₃)₂CuBr₄ (n=11-16) compounds compared to (CₙH₂ₙ₊₁NH₃)₂MCl₄ (M=Mn, Cu, Cd) compounds of the same lengths (Tables 2 to 4).

As anticipated, the atoms in the NA chains of (NA)₂CuBr₄ have much larger atomic displacement parameters in the LT phase than those in the DA chains of the LT phase of (DA)₂MnCl₄ (FIGS. 5E and 5G). This is consistent with the smaller increase in chain flexibility and number of rotatable C—C bonds modelled for conversion to the HT phase of (NA)₂CuBr₄. Note that NA chains adopt two conformations—alternating between chains with a gauche C₂-C₃ bond and those with a gauche C₁-C₂ bond, each modeled with two-part disorder—in the LT crystal structure of (NA)₂CuBr₄, and average displacement parameters are similar for both chain conformations. The residual motion and configurational disorder in the LT phase of (NA)₂CuBr₄ is further corroborated by variable-temperature IR spectra, which show a band near 1360 cm⁻¹ assigned to CH₂ wagging from gt₂ₙ₊₁g′-type kinks that is present below the phase transition temperature for (NA)₂CuBr₄ but is only present above the phase transition temperature for (DA)₂MnCl₄. The IR spectra also suggest that the local environment around the chain ends (CH₃) and headgroups (NH₃⁺) is more similar in the LT and HT phases of (NA)₂CuBr₄ than in those of (DA)₂MnCl₄ (Tables 7 to 8).

Although conformational disorder in the LT phase leads to a decreased entropy change, it also likely contributes to the enhanced reversibility of the (NA)₂CuBr₄ chain-melting transition through two primary effects. First, the higher degree of disorder in the alkylammonium chains in the LT phase-along with the softer nature of Br anions—should make the (NA)₂CuBr₄ lattice more compressible than the (DA)₂MnCl₄ lattice. Since the barocaloric coefficient dTₜᵣ/dP of a solid tends to increase with increasing compressibility (43, 44), this would be expected to make the phase transition in (NA)₂CuBr₄ more sensitive to pressure. Indeed, dTₜᵣ/dP for the transition to the LT phase of (NA)₂CuBr₄ is 29% higher than for (DA)₂MnCl₄. Second, the presence of certain configurational degrees of freedom, such as gt₂ₙ₊₁g′ kinks, in both the LT and HT phases should render the two phases more compatible, lowering both isobaric and isothermal hysteresis. In any case, both compounds display, near room temperature, large and reversible barocaloric cooling, represented by their materials properties Tₜᵣ, ΔSₜᵣ, and Pᵣₑᵥ, and are highly competitive with other leading barocaloric materials (FIGS. 7A and 7B, Tables 18 to 19).

In addition to its influence on operating pressure, hysteresis, which leads to dissipative heat losses, adversely impacts the second-law efficiency and coefficient of performance (COP) of any caloric cooling cycle. The impact of hysteresis on efficiency can be quantified by calculating the idealized thermodynamic efficiency, η, of a caloric material—relative to the Carnot efficiency—using a simple material model that integrates the dissipative losses in a Carnot-like cycle:

$$\\ η = \frac{C O P}{C O {P}_{Carnot}} = \frac{1}{1 + 4 \frac{Δ {T}_{hys}}{Δ {T}_{ad , max}}}$$

Based on this model, caloric materials with ΔT_{hys}/ΔT_{ad,max} of less than 10% will have second-law efficiencies competitive with those of conventional vapor compression-based systems (˜85%). Excitingly, (DA)₂MnCl₄ and (NA)₂CuBr₄ display second-law efficiencies of 88 and 93%, while the values found in most barocaloric compounds range between 40 and 65% (Table 19), either because of large ΔT_{hys} or a low ΔSₜᵣ that leads to a small ΔT_{ad,max}. Additionally, both compounds display the largest values of barocaloric strength—the reversible isothermal entropy change ΔS_{it,rev} normalized by the driving pressure—than have been realized for barocaloric materials (Table 19).

These results highlight exciting opportunities to exploit the tunability of 2-D perovskites to independently manipulate phase-change hysteresis, entropy, and sensitivity to pressure for improved barocaloric performance. For instance, it should be possible to realize chain-melting transitions with even higher entropy changes through functionalization of the organic bilayers—such as by introducing aromatic groups or hydrogen bond donor-acceptor pairs—and even lower hysteresis through modification of the organic-inorganic interface—such as by incorporating mixtures of different halide anions or introducing defects—or through leveraging multicaloric effects. In addition, the anisotropic nature of the chain-melting transition in (DA)₂MnCl₄ and (NA)₂CuBr₄—wherein an increase in interlayer spacing along a single direction accounts for ˜80% of the volume change-suggests that uniaxial stress, which can be readily applied through mechanical actuation, may be able to drive large elastocaloric effects in 2-D perovskites.

The results and materials discussed herein were obtained according to the following methods.

### EXPERIMENTAL METHODS

All manipulations were conducted in air unless otherwise noted. Anhydrous diethyl ether was obtained from a Pure Process Technology anhydrous solvent system. Anhydrous methanol and ethanol were purchased from a commercial vendor and used as received. All other reagents were purchased from commercial vendors and used as received. Single crystal diffraction data was collected using a Bruker D8, SMART APEX II, or APEX DUO instrument. IR spectra were obtained on a Bruker ALPHA II Platinum ATR with a variable temperature stage. Thermogravimetric analysis (TGA) experiments were performed using a TA Instruments TGA550. Abbreviation used: DA=decylammonium, (C₁₀H₂₁NH₃)₂MnCl₄=(DA)₂MnCl₄, (NA)=nonylammonium, (C₉H₁₉NH₃)₂CuBr₄=(NA)₂CuBr₄.

### Synthesis of Two-Dimensional Perovskites

(DA)₂MnCl₄·: Decylamine (≥99.0%) and hydrochloric acid (HCl) solution (37 wt %) were purchased from Sigma Aldrich and used without further purification. C₁₀H₂₁NH₃Cl was first synthesized by adding HCl solution (550 μL, 6.6 mmol) into decylamine (1.1 mL, 5.5 mmol) in ca. 5 mL ethanol in a cold-water bath. After evaporating the solvent at reduced pressure, the resulting white powder of (DA)Cl was washed with diethyl ether and vacuum dried at room temperature for a day. Crystalline powders of (DA)₂MnCl₄ were prepared by the cooling of an ethanol solution containing a stoichiometric quantity of the manganese(II) chloride and (DA)Cl, as previously reported (e.g., in M. R. Ciajolo, et al., Comparative Studies of Layer Structures: The Crystal Structure of Bis(Monodecylammonium)tetrachloromanganate(II). Gazzetta Chimica Italiana. 106, 807 (1976), and H. Arend, et al., Layer perovskites of the (CₙH₂ₙ₊₁NH₃)₂MX₄ and NH₃(CH₂)ₘNH₃MX₄ families with M=Cd, Cu, Fe, Mn or Pd and X=Cl or Br: Importance, solubilities and simple growth techniques. J. Cryst. Growth. 43, 213-223 (1978)). (DA)Cl (96.9 mg, 0.5 mmol) was dissolved in 4.0 mL of ethanol. After several minutes of stirring, MnCl₂·4H₂O (49.5 mg, 0.25 mmol) was added to the solution, and the solution was heated to 65° C. After the cooling this solution to room temperature at a rate of 4 K h⁻¹, pale pink crystals were obtained. The crystals were filtered and washed with diethyl ether (5×10 mL) and held at reduced pressure for 6 h to afford 45.2 g (35.2% yield) of product. Crystals suitable for structure determination were obtained by slow cooling at a rate of 2 K h⁻¹.

(NA)₂CuBr₄: Nonylamine (≥99.5%) and hydrobromic acid (HBr) solution (48 wt %, 8.8 M) were purchased from Sigma Aldrich and used without further purification. C₉H₁₉NH₃Br was first synthesized by adding HBr solution (545 μL, 4.8 mmol) into nonylamine (733 μL, 4.0 mmol) in ca. 5 mL ethanol in a cold-water bath. The solvent was removed at reduced pressure to yield colorless powder of (NA)Br. The powder was washed with diethyl ether and vacuum dried at room temperature for a day. CuBr₂ (402 mg, 1.8 mmol) and C₉H₁₉NH₃Br (807 mg, 3.6 mol) were dissolved in 2 mL of ethanol. The solution was slowly cooled from 65° C. to room temperature at a rate of 4 K h⁻¹. The saturated solution was then stored at below ambient temperature (5° C.) for 1 hour. The resulting dark purple precipitate was filtered and washed with diethyl ether (5×10 mL). The dark purple crystalline powder was held at reduced pressure for 12 h to remove moisture. Crystals suitable for structure determination was obtained by slow evaporation of a 1-mL solution of (NA)₂CuBr₄ (202 mg, 0.3 mmol) in methanol. Anal. Calcd. for (C₉H₁₉NH₃)₂CuBr₄: C: 32.19%, H: 6.60%, N: 4.17%, Br: 47.58%, Found: C: 31.84%, H: 6.69%, N: 4.43%, Br: 47.76%.

### Differential Scanning Calorimetry (DSC)

DSC at ambient pressure: A Discovery 2500 DSC with an RCS 90 cooling system (TA Instruments) was used to measure the transition temperatures and gravimetric enthalpies for all compounds. The DSC baseline and cell thermal parameters were calibrated using sapphire discs. The temperature and cell constant were calibrated using an indium standard. All DSC samples were prepared in air using 3-15 mg of sample and were hermetically sealed in aluminum pans (purchased from TA instruments). The sample was scanned under a dynamic flow of N₂ (50 mL min⁻¹). An empty, hermetically sealed aluminum pan we used as a reference.

Determination of Tₜᵣ and gravimetric ΔHₜᵣ and ΔSₜᵣ: Transition temperatures, Tₜᵣ, and enthalpies of transition, ΔHₜᵣ, were determined using the TA Instrument TRIOS or Netzsch Proteus software. Peaks were selected for analysis by defining a temperature range containing the peak of interest. The lower bound and upper bounds of the temperature range were chosen to encompass the phase transition, which starts with a deviation from the baseline and ends with a return to baseline.

Prior to determination of Tₜᵣ or ΔHₜᵣ, a baseline, which models the heat flow in the absence of transition, must be generated to approximate the baseline in the transition region in the absence of a transition. A baseline is generated within the defined temperature range using various option that determine the slope of the lower and higher temperature limits and shape of the baseline. When possible, baselines were generated using mutual tangent slopes at both the upper and lower temperature limits with a sigmoidal baseline, which we found to produce most physically reasonable baselines.

The extrapolated onset temperature was reported as the transition temperature, as is standard in DSC data analysis, because the onset temperature—unlike the peak temperature—is relatively independent of experimental parameters like the heating rate or sample mass. The onset temperature is determined by identifying the region of the onset melting peak that has the highest slope, defining a tangent to that region, and then extending the tangent to the generated baseline. The intersection between the baseline and the tangent is the onset temperature. Endotherms were integrated between the upper and lower temperature limits with the baseline subtracted to provide ΔHₜᵣ, and ΔSₜᵣ was calculated through ΔSₜᵣ=ΔHₜᵣ/tₜᵣ. If physically reasonable limits were chosen, the onset transition temperatures and ΔHₜᵣ did not depend strongly on the choice of the temperature limits, and such variations were within the error of the measurements, which is estimated to be <0.5% for Tₜᵣ and <2% for ΔHₜᵣ. Note that volumetric ΔHₜᵣ were calculated from gravimetric quantities using crystallographic densities at ambient temperature.

DSC at applied hydrostatic pressure: High-pressure DSC measurements at the pressure range between 1 to 150 bar were carried out in a DSC 204 HP Phoenix® (Netzsch). The temperature and heat flow were calibrated at each pressure using an indium standard. The temperature and cell constant were calibrated at each pressure using an indium standard. Helium gas was used as a pressure-transmitting medium. All DSC samples were prepared in air using 3-10 mg of sample and were sealed in aluminum pans (purchased from Netzsch) with a pierced lid. An empty, aluminum pan with a pinhole was used as a reference. All measurements were carried out in a dynamic gas environment with a 50 ml min⁻¹ He. Otherwise noted, heating and cooling rates of 2 K min⁻¹ were used during isobaric measurements.

During the pressure cycling experiments, heat flow signals were measured over time under the repeated application and removal of a hydrostatic pressure of 150 bar at 311 K for (DA)₂MnCl₄ and 307 K for (NA)₂CuBr₄. The pressure linearly increased at a rate of 6 bar min⁻¹ and asymptotically decreased at an average rate of 13 bar min⁻¹. During the pressure change, quasi-isothermal conditions were maintained, where a small change in temperature (<1 K) induced by gas compression and decompression is compensated by external thermal control measures. The pressurization and depressurization processes are associated with average temperature fluctuations of 0.3 K and 0.7 K, respectively. To distinguish the heat flow signals associated with pressure-induced phase transitions of samples from those associated with compression and decompression of pressure transmitting medium (He gas), (C₁₂H₂₅NH₃)₂MnCl₄, prepared from the previously reported procedure (G. F. Needham, et al., J. Phys. Chem. 88, 674-680 (1984), was used as a blank sample because it undergoes transitions at temperatures (T_{tr, major}=331 K and T_{tr,minor}=335 K) well above the set temperatures for (DA)₂MnCl4 and (NA)₂CuBr₄. The heat flow signals measured from the blank during the pressure change at the set temperature were modeled as a baseline for the sample data. By subtracting the features in the heat flow associated with the gas compression and decompression from the sample data, we were able to determine the onset pressure associated with the pressure-induced transitions during compression and decompression processes. Because maintaining isothermal conditions becomes challenging once the phase transition is induced and both pressure and temperature change drive the endothermic and exothermic transitions to completion, we did not integrate the heat flow signals and focused only on using the information to determine the onset pressures for transitions.

On the barocaloric effects outside the transition: We note that the ΔSᵢₜ values obtained through our HP-DSC experiments do not include the contributions from the additional barocaloric effects (ΔS₊) that arise from each phase. This additional contribution can be estimated through ΔS₊=−[(∂V/∂T)ₚ₌₀]ΔP, where ΔP and (∂V/∂T)ₚ₌₀ denote a driving pressure and a thermal expansion at the ambient pressure, respectively. Note that the isothermal entropy contribution is derived from the Maxwell relation (∂V/∂T)ₚ=−(∂V/∂P)_{T} with an assumption that the volume expansion is independent of pressure. The ΔS₊ values are estimated to be ˜3 and ˜4 J kg⁻¹ K⁻¹ at the ordered and disordered phases, respectively, under 150 bar driving pressures. Although these values are small in comparison with ΔSₜᵣ, these contributions can be large at higher driving pressure, such as ˜# at 400 bar, because of large thermal expansion coefficients (˜10⁻⁴ K⁻¹)

### Helium Pycnometry

Sample density was determined using an InstruQuest Inc. μ-ThermoPyc variable temperature He pycnometer. In a typical measurement, ca. 150 mg of crystalline sample were transferred to the sample holder, and the sample mass obtained. The holder was then placed in the instrument test chamber and the headspace evacuated and refilled five times to obtain a pure He atmosphere. The sample was then cycled multiple times through the order-disorder transition with the chamber volume determined every 2-5° C. away from the transition and every 0.5-1.0° C. close to the transition. For each point, the temperature was fully equilibrated with a standard deviation of no more than 0.2° C. prior to volume measurement. At each temperature, the chamber volume was measured five times to obtain good statistics. Sample volume was then determined by subtracting the average observed chamber volume from the volume of the empty sample holder which had been measured previously. The sample mass was redetermined after the measurement and found to have decreased by no more than 0.5 mg, likely due to loss of adsorbed water. Uncertainties of the reported densities were determined by propagation of the standard deviations of the empty and filled chamber volumes and the sample mass.

### X-Ray Crystallography

X-ray diffraction analyses were performed on a single crystal coated with Paratone-N oil and mounted on a MiTeGen microloops, at different temperatures (100 to 335 K) controlled by an Oxford Cryostreams nitrogen flow apparatus. Crystals were mounted at 270 K, and 270 K data sets were collected. Crystals were then cooled to 100 K for 100 K data collection. After 100 K data sets, high-temperature data sets were collected, 330 K for (DA)₂MnCl₄ and 335 K for (NA)₂CuBr₄. The temperature was manipulated at a rate of 60 K h⁻¹. The intensities of the reflections were primarily collected by a Bruker D8 diffractometer with CMOS area detector (Mokα radiation, λ=0.71073 Å). The collection method involved 0.5° scans in ω at 23° in 2θ° with a detector distance at 9 cm for (DA)₂MnCl₄ and 8 cm for (NA)₂CuBr₄. Data integration down to 0.84 Å resolution was carried out using SAINT V8.37A with reflection spot size optimization (9). Most crystals were either single or merohedrally twinned and absorption corrections were made with the program SADABS. All single crystal structures were solved by the Intrinsic Phasing methods and refined by least-squares methods again F² using SHELXT-2014 and SHELXL-2018 with OLEX 2 interface. Thermal parameters were refined anisotropically for all non-hydrogen atoms. Hydrogen atoms were placed geometrically and refined using a riding model for all structures. Crystal data as well as details of data collection and refinement are summarized in Tables 15 and 16, and geometric parameters are shown in Tables 10 to 14.

Notes on data quality, twining, and disorders: The phase transition from low-temperature phase to high-temperature (HT) phase during heating often resulted in fracturing and twining of the crystals, giving rise to the decay of crystal quality. Due to the large thermal motions of the long alkyl ammonium cations, the geometric parameters calculated for the HT phase structures should be treated as estimates, as pointed out in the literature.

### Synchrotron In Situ Powder X-Ray Diffraction (PXRD) Studies

Powder X-ray diffraction data for (DA)₂MnCl₄ and (NA)₂CuBr₄ were collected on beamline 17-BM-B at the Advanced Photon Source (APS) at Argonne National Laboratory. All X-ray wavelengths were between 0.24 Å and 0.45 Å, and are specified for each experiment in the relevant figures and tables. For variable temperature and pressure experiments, approximately 10 mg of samples was loaded into a sapphire capillary (1.524 mm×1.07 mm×50.8 mm, Saint-Gobain Crystals). Each capillary was attached to a custom-designed flow cell equipped with a gas valve, which was mounted onto the goniometer head and connected to a syringe pump that the applied the hydrostatic pressure of Helium (80-360 bar). Sample temperature was controlled by an Oxford Cryostream (Oxford Cryostream 800+). Prior to measurement, samples were evacuated by flowing ambient pressure of He gas for 10 minutes and annealed by heating 20 K above Tₜᵣ and cooling back to ambient temperature, at a rate of 6 K min⁻¹ in the cryostream. The internal sample temperature was monitored via a K-type thermocouple (TC) that maintained the thermal contact with the powder sample within the capillary. Otherwise noted, the samples were heated and cooled by the cryostream at a rate of 6 K min⁻¹, which resulted in the rate of ca. 3 K min⁻¹ in the TC temperature due to the temperature gradient. Diffraction patterns were analyzed using the software TOPAS-R (Bruker AXS, version 3.0, 2005). Diffraction patterns at select temperature were indexed, and Le Bail refinements were performed to extract unit cell parameters.

### Conformational Disorder in Two-Dimensional Metal-Halide Perovskites

Conformational entropy: For an alkylammonium chain CₙH₂ₙ₊₁NH₃⁺, there are n-2 rotatable C—C bonds that can contribute to formation of different conformers. Note that the conformation of alkylammonium chains can be described through a sequence of dihedral angles often referred to as Hoffmann's notation, with the terms g⁺, g⁻, and t denoting dihedral angles of approximately +60 (gauche), −60 (gauche), and 180° (trans), respectively. In principle, each C—C bond should be able to equally access these three rotameric states that correspond to local energy minima, and the change in solid-state configurational entropy can be described as ΔS_{configuration}=R In W, where W is the ratio between the number of configurations in the disordered and ordered chains (That is, W=N_{disorder}/N_{order}). Thus, the entropy change associated with the conformational disordering of an alkylammonium chain can be estimated as R In 3ⁿ⁻².

However, depending on how chains are packed and how neighboring chains influence one another in each phase, the average number of “accessible” rotameric states for each C—C bond—defined as the chain flexibility number ϕ—can deviate from 3. Note that, in linear organic molecules, the flexibility number depends on the energetic difference between each conformer and a flexibility number of 2.85 has been used to predict the melting thermodynamics. In two-dimensional perovskites, a few structural features—steric restrictions (imposed by halide pockets and neighboring chains), correlations between torsions, and residual degrees of freedom present in C—C bonds in the ordered phase—can partially limit conformational degrees of freedom, reducing the number of C—C bonds associated with the chain melting processes by a restriction parameter β. The number of accessible conformations at the disordered phase can then be approximated as (ϕ)^{n-2-β}, which includes the conformers arising from kink {gtg′} formation and cooperative torsion along the chain axis. Here, we assume that the two parameters ϕ and β are independent. Note that the entropy change associated with a reorientational motion (flipping) of the entire alkylammonium chains between two energetically equivalent orientations within the metal-halide pocket can be accounted for by adding the term R In 2. This chain-flipping can occur as an isolated minor transition or be coupled to the major order-disorder transition. In addition to the flipping, the major conformation disordering can be often distributed across multiple, successive transitions, and the total number of structural transitions of a compound is here referred to as m. The contributions from both flipping (ΔS_{flipping}) and chain melting (ΔS_{melting}) to the total entropy change, ΔSₜₒₜₐₗ, are multiplied by 2, because 2 mole of CₙH₂ₙ₊₁NH₃⁺ chains are present in 1 mole of perovskite. The contribution from any change in entropy associated with the MX₆ octahedra is assumed to be negligible. Thus, the total difference in entropy associated m structural transitions in 2-D perovskites between low-temperature (LT) “ordered” and high-temperature (HT) “disordered” phases can be expressed through the following relationship:

ΔSₜₒₜₐₗ=Σᵢ₌₁^{m}ΔS_{tr,i}=ΔS_{flipping}+ΔS_{melting}=2RIn2+2RIn(ϕ)^{n-2-β} (1)

By fitting ΔSₜₒₜₐₗ as a function of chain length n, we were able to determine the flexibility number ϕ and the restriction parameter β for two-dimensional perovskite (CₙH₂ₙ₊₁NH₃)₂MX₄ (M=Mn, Cu, Cd; X=Cl, Br). ΔSₜₒₜₐₗ values are tabulated in Tables 2 to 4, and fitting parameters are summarized in Table 6. With the expectations that odd and even chain lengths should exhibit different thermodynamic trends, odd-numbered and even-numbered analogs were fit separately. We note that, somewhat surprisingly, the structural origins of odd-even effects of the phase-change thermodynamics in 2-D perovskites are not fully understood and require further investigations.

(DA)₂MnCl₄: From the fitting parameters (ϕ=3.0 and β=2.1, R²>0.99) for (CₙH₂ₙ₊₁NH₃)₂MnCl₄, (n=8-15), we can estimate that each decylammonium chain can access 3^{5.9} (˜650) distinct configurations as a result of conformational disorder at the HT phase. In addition, the total entropy change associated with the single-step transition at 310 K can be calculated as ΔS_{total,calc}=ΔS_{flipping}+ΔS_{melting}=2R In 2+2R In (3)^{5.9}=119 J K⁻¹ mol⁻¹, which agrees with the experimentally measured ΔS_{total,exp} of 118 J K⁻¹ mol⁻¹. This estimation is further supported by the single-crystal structure of (DA)₂MnCl₄ at the HT phase (330 K), where each alkylammonium chain is disordered over two energetically equivalent positions and has a conformation with six C—C dihedral angles of 150-166° that deviate from the ideal trans dihedral angle of 180° and two C—C dihedral angles (C1-C2, 174°; C3-C4, 180°) close to the trans angle. (Table 13). This deviation may indicate fast “trans-gauche” rotations around C—C bonds and/or cooperative torsion along the chain axis. It is worth emphasizing that the chain length dependence of ΔSₜₒₜₐₗ does not display a pronounced odd-even effect in 2-D Mn—Cl perovskites.

We also note that the conformational disordering is likely to be associated with the formation of one {gtg′} kink per chain (on average) near the chain ends and the most probable conformer is {t₄gtg′t}, as suggested by incoherent neutron scattering experiments and intramolecular energy calculations. At higher temperature, some chains could adopt energetically less stable forms, in which the kink defects are located near the polar head groups, such as {t₃gtg′t)} and {t₂gt₃g′t}. These proposed conformers are shown in the conceptual illustration in FIG. 3A. Note that the change in interlayer distance measured from X-ray diffraction experiments also indicates that each chain is likely to favor the formation of one kink on average. More specifically, when chains are positioned perpendicular to the metal-halide layers, the formation of a kink within a chain requires additional lateral space and reduces its projected chain length by 1.27 Å. Thus, the changes in interlayer distance, when combined with measurements on vibrational and dynamics of the chains, provide information about chain conformations.

The single-crystal structure at the HT phase also supports the proposed model that formation of kink is favored near the chain ends. In FIG. 5E-, equivalent isotropic displacement parameters (U_{equiv}) of N and C atoms in the decylammonium chain are shown for both LT and HT phases. Note that the atomic displacement parameters obtained from the crystal structure refinement process represent how the atoms deviate from their equilibrium positions and contain information about residual motion (such as rotations and vibration) and static, configurational disorder. The transition from LT phase to HT phase results in a large increase in U_{equiv} values, and the magnitude of the increase in U_{equiv} increases from the NH₃-polar head to the methyl end group along the chain. This result indicates that the chain melting transition gives rise to a large increase in dynamic disorder, and there is a gradient of disorder along the chain, which also supports that the kink formation is favored near the chain end. The gradient of dynamic disorder, which was also observed in the LT phase data as well, likely originates from the difference in intermolecular interactions between the polar chain head (charge-assisted hydrogen bonding) and the methyl end (weak van der Waals interactions).

(NA)₂CuBr₄: We note that the transition entropy of (NA)₂CuBr₄ is about 48% and 56% of ΔSₜₒₜₐₗ of (C₉)₂MnCl₄ and (C₉)₂CuCl₄, respectively. This trend is also observed in the previously reported thermal data of (Cₙ)₂CuBr₄ (n=11-16), which shows that the transition entropies of 2-D Cu—Br perovskites are only 40-60% of those reported in Cu—Cl and Mn—Cl analogs. Fitting ΔSₜₒₜₐₗ with chain length n results in ϕ=2.1 and 2.2 and β=5.1 and 5.5, for odd-numbered and even-numbered chains, respectively (Table 6). The lower value of ϕ and higher value of β, compared to those obtained from Mn—Cl and Cu—Cl series, indicate that the difference in solid-state conformational entropy between LT and HT phases is smaller in (Cn)₂CuBr₄, and the difference likely originates from the smaller difference in the flexibility of the chain and the number of newly rotating C—C bonds.

The LT phase crystal structure indicates that the smaller difference in solid-state entropy between LT and HT phase may result from the higher degree of disorder present in the nonylammonium chains at the LT phase. In the LT phase, each of the two chain conformations (chain A with C1-C2 gauche bond and chain B with C2-C3 gauche bond) is modeled with two-part disorder (Table 14). Note that the atomic positions in chain A and chain B were refined to 35/65% and 53/47% occupancies, respectively. The analysis on the chain conformations shows that (i) the chains are distorted near the methyl ends with C7-C8 dihedral angles of and +159°/−170° in chain A (Part 1/Part 2) and +164°/−164° in chain B and (ii) the chain A displays additional distortion in the C3-C4 bond (−159°/+169°). These results illustrate that configurational disorder is present in the LT phase of (NA)₂CuBr₄. In addition, the chains also display U_{equiv} values higher than those in decylammonium chain of (DA)₂MnCl₄ at the LT phase, whereas the U_{equiv} values at the HT phase were similar in both compounds (FIGS. 5E and 5G). In addition, there is a gradient of disorder along the nonylammonium chain, similar to that present in (DA)₂MnCl₄. As a result, the difference in U_{equiv} values between LT and HT phase is smaller in (NA)₂CuBr₄. These trends collectively demonstrate that the higher degree of disorder present in nonylammonium chains at the LT phase—which result from both configurational disorder and residual motion—give rise to the lower ΔSₜᵣ in (NA)₂CuBr₄ compared to those in M-Cl analogs. We also note that the existence of 2^{nd} order phase transition can be ruled out based on the previously reported heat capacity measurements on the 2-D Cu—Br perovskites (25).

Comparison with 2-D Cd—Cl and Cu—Cl perovskites. Although the chain length dependences of ΔSₜₒₜₐₗ in 2-D Cd—Cl and Cu—Cl perovskites exhibit similar trends to that observed in the Mn—Cl series, with ϕ˜3 and β˜2, the trends slightly deviate from linearity and/or display pronounced odd-even effects. Even with the same chain length, these analogs display different phase transition behaviors, depending on the identity of metals. For example, unlike (C₁₀)₂MnCl₄, both (C₁₀)₂CuCl₄ and (C₁₀)₂CdCl4 display two-step transitions, and the major transition in (C₁₀)₂CuCl₄ is followed by a minor transition, whereas the major transition in (C₁₀)₂CdCl4 is preceded by a minor transition. These compounds also display differences in chain conformations at room temperature: (C₁₀)₂MnCl₄ with B conformer (C2-C3 gauche), (C₁₀)₂CdCl₄ with both A conformer (C1-C2 gauche) and B conformer), and (C₁₀)₂CuCl₄ with A, B, and all trans {t₈} conformers. As expected, these changes translate to the most probable conformational disorder at the HT phase. Overall, these observations indicate that (i) the trends in thermodynamics of chain melting transitions are sensitive to chain packing and metals and (ii) the stepwise conformational disordering transitions observed in most compounds are complex. To fully describe the trends and the impact of the phase transition behaviors on barocaloric effects, detailed investigations into the structural changes and microscopic motions associated with each transition are required, both at ambient and applied pressures.

Comparison with melting of n-alkane: For the melting transition of n-alkane and solid-solid transitions of 2-D metal-halide perovskites and related systems, the relationships among transition entropy (ΔSₜᵣ), chain length (n), and temperature of transition (Tₜᵣ) have been investigated, where ΔSₜₒₜₐₗ normalized by chain length n is fit with Tₜᵣ. Although this approach, often referred to as SnT analysis, can provide insights into the trends in series of 2-D perovskites and related compounds, we did not include the temperature of transition as a fitting parameter because most 2-D perovskites undergo stepwise transitions and the nature of molecular motions associated with each transition is not fully understood. However, both approaches provide similar insights into the conformational degrees of freedom of alkylammonium chains of 2-D perovskites during the phase transition. For example, the SnT analysis indicates that the total transition entropy increases by 9.1 J K⁻¹ mol⁻¹ per carbon in (Cₙ)₂MnCl₄ (n=7, 11-17) and 13.5 J K⁻¹ mol⁻¹ per carbon in n-alkane. The chain length dependence of 9.1 J K⁻¹ mol⁻¹ in 2-D Mn—Cl perovskites can be translated to the flexibility number of 3, which agrees with our analysis. As previously pointed out, the ratio between the chain length dependences of ΔSₜₒₜₐₗ in (Cₙ)₂MnCl₄ and n-alkane is 2:3 and correlates to the ratio between their dimensionalities. This interesting relationship is supported by a theoretical prediction based on kink-block transitions, where the melting entropy of alkyl chains confined in two-dimensional layers was shown to exhibit similar chain length dependence of 9.1 J K⁻¹ mol⁻¹. We also note that this value is close to the pressure dependences of melting transitions of n-decane (21 K kbar⁻¹) and n-nonane (20 K kbar⁻¹) (29). (nonane, with a dTdP solid-solid transition also similar).

Notes on dynamics: In addition to the kink formation (gauche defect), the cooperative torsion along the chains is coupled to the overall molecular motion at the HT phase. According to incoherent neutron scattering experiments, molecular motion corresponding to kink formation and cooperative torsion occurs over 1-5 ps timescale. However, due to the relatively low Q-values explored in the measurements, overall molecular motions, cooperative torsions, and kink formations were not accurately distinguishable. We also note that further studies are needed to fully model the microscopic details of the chain melting processes, both for fast motions (e.g., kink motion within a chain) and slow processes (e.g., the formation of “clusters” with similar chain conformations), at ambient and applied pressures.

Differences in phase transition behaviors between (DA)₂MnCl₄ and (DA)₂CdCl₄: First of all, their transition behaviors are qualitatively similar, with the chains at HT phase adopting approximately one kink conformation per chain. Neutron scattering experiments indicates, however, that the diffusion of kink, which was previously observed in (C₁₀)₂CdCl₄ with ˜1000 ps time scale by proton NMR and ³⁵Cl and ¹⁴N quadrupole resonance spectroscopies, is unlikely to occur in (C10)₂MnCl₄, because no cooperative conformational interconversion within a chain with a time scale greater than 20 ps was observed. This indicates that, unlike in (C₁₀)₂CdCl₄, in (C₁₀)₂MnCl₄, some kink formations are energetically more dominant. For the diffusion of the kinks to occur, several conformers with similar energies need to be in equilibrium. From the spectroscopic studies, it was revealed that (DA)₂CdCl₄ does have multiple conformers with similar energy levels. We also note that vibrational studies revealed that {tgtttg′tt} is the most probable conformer in (C₁₀)₂CdCl₄, which is more flexible in the middle of the chain and does not have an end-gauche conformation.

### Infrared Spectroscopy Analysis

Note that the IR signals used for conformational analysis are summarized in Tables 7 and 8.

C—H stretching: In the compounds containing long alkyl chains, shifts in C—H stretching peaks to higher wavenumbers are correlated with an increase in the number of gauche C—C bonds, a change in chain packing, and presence of order-disorder transitions. For example, in the melting transitions of n-alkanes, v_{symmetric}(C—H) and v_{anti-symmetric}(C—H) shift from 2920 to 2928 cm⁻¹ and 2850 to 2856 cm⁻¹, respectively. 2-D perovskites also display similar trend, with the shifts Δv of 2-3 cm⁻¹. The temperature dependence of these peaks can be either abrupt or gradual depending on metal and chain packing. Overall, this feature provides indirect evidence on the changes in the disorder in 2-D perovskites.

CH₂ rocking and bending: For well-ordered chains in a monoclinic or orthorhombic lattice, CH₂ rocking and bending bands split into doublets near 720 and 1470 cm⁻¹, respectively, because of the factor group splitting that arises from directional intermolecular interactions between the chains. Generally, these features have been observed across materials with long hydrocarbon chains, including n-alkanes, layered silver thiolates, and 2-D metal-halide perovskites. The frequencies, shapes, and separations of these signals are correlated with orientations between neighboring chains and conformational disorder within the chain. In 2-D perovskites, the disappearance of the splitting can be used to determine if the chain undergoes conformational disordering, because it is correlated with the emergence of CH₂ wagging bands specific to defect conformations (e.g., gtg′ kink) and indicative of the re-orientational motion of whole chain. However, we note that understanding specific microscopic motions associated with these features requires further investigations.

CH₂ wagging: CH₂ wagging bands, which provide insights into vibrational modes localized on a few CH₂ units pinned in specific conformation sequences, are weakly coupled with the host lattice and independent of chain length. Thus, they provide characteristic signals of specific conformational defects. In 2-D perovskites, these modes are readily mixed with internal modes of chain end (CH₃) and head (NH₃⁺), and this coupling enhances the intensity of some CH₂ wagging modes. Through normal-mode calculations on (C₁₀)₂CdCl₄ and related compounds, the key peaks have been assigned. These signals appear at 1310 cm⁻¹ (in-plane, two CH₂ units, kink), a shoulder near 1370 cm⁻¹ (out-of-plane, two CH₂ units, kink), 1350 cm⁻¹ (gg conformation), 1340 cm⁻¹ (tg conformation near the chain end). The kink refers to gt₂ₙ₊₁g′-type conformational defects.

NH₃ and CH₃ bending: In 2-D M-Cl perovskites, the signal from NH₃ symmetric bending modes appears around 1490 cm⁻¹ at the LT phase and are often split into doublet due to the crystal field effect. The degree of the peak splitting highly depends on the identity of metals, with Mn—Cl and Cd—Cl perovskites displaying very small and often negligible splitting and Cu—Cl perovskites showing clear doublet (1492-1480 cm⁻¹). At the HT phase, the peak splitting disappears, often accompanied by noticeable broadening, and this indicates the reorientational motion of the polar head within the halide pocket. The NH₃ antisymmetric bending mode is associated with a strong singlet peak near 1580 cm⁻¹ and red-shifts by 6-8 cm⁻¹, as a compound undergoes a structural transition. This feature can be correlated with the decrease in the strength of N—H . . . Cl hydrogen bond. As these features are associated with the onset of chain melting transitions, vibrational spectra from NH₃ bending can provide insights into the motions of chains within the halide pocket. The CH₃ symmetrical bending mode, which appears near 1376 cm⁻¹ at the LT phase, blue-shifts (Δv˜3 cm⁻¹) as the chains undergo structural transitions. This feature is correlated with the change in inter-lamellar interactions within the organic bilayers.

Comparison of vibrational spectra related to chain conformations: IR spectra of (DA)₂MnCl₄ and (NA)₂CuBr₄ are summarized in FIGS. 10A-10D. In (DA)₂MnCl₄, CH₂ rocking and bending signals at ˜720 and ˜1470 cm⁻¹ are doublet at the LT phase, due to the factor group splitting that arises from directional, inter-chain interaction in the monoclinic unit cell (FIGS. 10B an 10D). The splitting disappears at the HT phase as a result of chain disordering. In (NA)₂CuBr₄, however, the factor group splitting is not observed, presumably because the chains are arranged in a triclinic unit cell (FIGS. 10A-10D). Both CH₂ rocking and bending bands appear at the frequencies similar to those of (DA)₂MnCl₄ and do not display a noticeable change in peak shape after transition. Thus, these signals do not provide useful information about the difference in the disordering processes of the alkylammonium chains.

For symmetric C—H stretching peaks, both compounds display blue-shifts (˜2 cm⁻¹) after the transitions, which supports that the phase transition introduces disorder in the alkylammonium chains (FIG. 10A). Symmetric CH₃ bending peaks can also provide insights into the difference in molecular motions of both compounds at each phase, as the layer-layer interactions between the chain ends within the organic bilayer are also correlated with the chain disorder. Although the peaks blue-shift in both compounds, the degree to which the peak shifts is smaller in (NA)₂CuBr₄ (Δv˜1 cm⁻¹) than in (DA)₂MnCl₄ (Δv˜4 cm⁻¹) (FIG. 10B). The peak shifting in (NA)₂CuBr₄ was even smaller than those measured in Cu—Cl analogs (Δv ˜2 cm⁻¹), as shown in Table 8. This result indicates the difference in the local environments around the methyl ends are smaller in (NA)₂CuBr₄ than in (DA)₂MnCl₄.

Both compounds show pronounced differences in the progression of CH₂ wagging bands, as shown in FIG. 10C. In (DA)₂MnCl₄, CH₂ wagging bands associated with {gt₂ₙ₊₁g′} kink formation emerge near 1310 and 1367 cm⁻¹ at the HT phase. The appearance of these peaks is also accompanied by broadening and decrease in intensity of a few other bands associated with other wagging/twisting motions in the range (1400-1300 cm⁻¹). In addition, no signal at 1350 cm⁻¹ was observed, which indicates that the highly distorted {gg} conformation is not formed at the HT phase. Note that these trends are similar to those observed in (C₁₄)₂MnCl₄. Interestingly, (NA)₂CuBr₄ displays a very different trend. At the LT phase, a shoulder peak at 1360 cm⁻¹ (near the CH₃ symmetric bending peak at 1378 cm⁻¹) is observed, which indicates that conformational disorder associated with a kink conformation is present even before the transition. At the HT phase, this shoulder peak disappears while another CH₂ wagging band, also associated with a kink formation, emerges at 1312 cm⁻¹. This result suggests that the compound, through the phase transition, undergoes a switching in most favorable conformers, each of which contains a kink. The signal associated with {gg} conformation (near 1350 cm⁻¹) is also not detected. In addition, a peak near 1340 cm⁻¹ at the LT phase disappears after the transition, which may indicate the existence of an end-gauche conformation before the transition. In addition, the phase transition in (NA)₂CuBr₄ does not seem to have much impact on the peak shape in the CH₂ wagging region (1400-1300 cm⁻¹). We also note that the possibility of the end-gauche conformer in (NA)₂CuBr₄ is supported by its smaller shift in symmetric CH₃ bending peaks and the higher frequency of the LT phase peak (1378 cm⁻¹) than vₛ(CH₃)_{bending} of (DA)₂MnCl₄ at 1375 cm⁻¹ at the LT phase.

Taken together, these results indicates that (i) the differences in conformations of and local environments around the chains in (NA)₂CuBr₄ is smaller than those in (DA)₂MnCl₄ and (ii) a noticeable degree of conformational disorder is present in (NA)₂CuBr₄ at the LT phase. These results, qualitatively, agree with our interpretations of single-crystal structures at the HT phase. Although the trends we observed from both compounds are consistent with those measured in other 2-D metal-halide perovskites as shown in Table 8, we note that accurate assignments and quantitative interpretation of IR spectra, particularly of CH₂ wagging bands, are challenging, because the signals are relatively weak, coupled with the internal modes of chain end and head, and sensitive to the positions and diffusion of the defects, with the relationships among these factors not well understood. For accurate assignments of chain conformations, particularly those for (NA)₂CuBr₄, further investigations using normal-mode calculations and other complementary spectroscopic techniques, such as Raman or sum frequency generation vibrational spectroscopy, will be required.

Chemical origin of the difference in the solid-state disorder between (NA)₂CuBr₄ and (DA)₂MnCl₄: The difference in the disorder between the two compounds may arise from the difference in the size of metal-halide pocket and the strength of N—H . . . X (X=Cl, Br) hydrogen-bond interactions within the pocket. In particular, comparisons of IR spectra associated with NH₃ bending modes provide useful insights into how the charge-assisted H-bond interactions differ between the two compounds. As shown in FIGS. 10A and 10B, the anti-symmetric NH₃ bending mode of (NA)₂CuBr₄ appears at 1570 cm⁻¹ which is 15 cm⁻¹ lower than that of (DA)₂MnCl₄ (1585 cm⁻¹), as well as lower than other M-Cl perovskite analogs (Cu—Cl, 1583 cm⁻¹; Cd—Cl, 1589 cm⁻¹). In (NA)₂CuBr₄, the position and shape of vₐₛ(NH₃)_{bending} peak does not change after the transition, whereas (DA)₂MnCl₄ undergoes both noticeable red-shifting (Δv˜−6 cm⁻¹) and peak broadening through the transition. This result suggests that the polar heads (NH₃⁺) in the decylammonium chains displays re-orientational motions accompanied by the weaking of hydrogen bonds, while the local environments around those in the nonylammonium chains remain nearly unchanged after the transition. We also note that the vₐₛ(NH₃)_{bending} peak of (NA)₂CuBr₄ displays a very small splitting at the LT phase, which may suggest that its A and B conformers experience slightly different hydrogen bonds at the Cu—Br pockets. Similar trends are observed in the NH₃ symmetric bending modes: the vₛ(NH₃)_{bending} peak of (NA)₂CuBr₄ was red-shifted by ˜16 cm⁻¹ compared to that of (DA)₂MnCl₄ and the transition-induced peak broadening is observed only in (DA)₂MnCl₄. Note that we do not discuss the peak splitting of vₛ(NH₃)_{bending} peaks, because (i) the degree to which the peak splits in (DA)₂MnCl₄ was very small and (ii) vₛ(NH₃)_{bending} peak of (NA)₂CuBr₄ overlaps with its CH₂ bending peak at 1470 cm⁻¹.

Collectively, these results illustrate that, for (NA)₂CuBr₄, the weaker N—H . . . Br hydrogen bonds, in combination with a larger area provided for each chain (from incorporation of larger Br ions and presence of Jahn-Teller distortion), result in the increased degrees of freedom of chains in (NA)₂CuBr₄ at the LT phase, which contributes to the difference in the chain disorder between LT and HT phase smaller than the difference in (DA)₂MnCl₄. We also hypothesize that the smaller thermal hysteresis observed in (NA)₂CuBr₄ may arise from this enhancement in the conformational degrees of freedom, as it may lower the energy barrier associated with the formation of nucleation sites (i.e., “clusters” with similar chain conformations). The structure-property relationship between the two compounds provides insights into how conformational disorder in the 2-D perovskites can be controlled through chemical manipulations of the organic-inorganic interfaces.

### Data tables

TABLE 2
Summary of previous reported chain melting transitions in representative
two-dimensional (CₙH₂ₙ₊₁NH₃)₂MnCl₄ perovskites.

| | | | ΔH | ΔH | ΔS | ΔS |
|---|---|---|---|---|---|---|
| Chemical | | Tₜᵣ^{c} | (kJ | (kJ | (J mol⁻¹ | (J kg⁻¹ |
| Formula^{a} | Type^{b} | (K) | mol⁻¹) | kg⁻¹) | K⁻¹) | K⁻¹) |
| (C₆)₂MnCl₄ | minor | 206 | 5 | 13 | 26 | 64 |
| | major | 291 | 10 | 25 | 37 | 93 |
| | total | | 15 | 38 | 63 | 157 |
| (C₇)₂MnCl₄ | major | 250 | 17 | 39 | 68 | 159 |
| | minor | 314 | 10 | 24 | 33 | 76 |
| | total | | 27 | 63 | 101 | 235 |
| (C₈)₂MnCl₄ | major | 274 | 19 | 42 | 70 | 153 |
| | minor | 302 | 4 | 10 | 14 | 32 |
| | total | | 24 | 52 | 84 | 185 |
| (C₉)₂MnCl₄ | major | 291 | 26 | 53 | 89 | 183 |
| | minor | 294 | 2 | 5 | 8 | 16 |
| | total | | 28 | 58 | 97 | 199 |
| | | 287^{e} | 31 | 63 | 107 | 220 |
| (C₁₀)₂MnCl₄ | | 308 | 35 | 68 | 113 | 221 |
| | | 310 | 37 | 72 | 118 | 230 |
| (C₁₁)₂MnCl₄ | major | 317 | 40 | 74 | 126 | 234 |
| | minor | 321 | 4 | 8 | 13 | 25 |
| | total | | 44 | 82 | 140 | 258 |
| | | 316^{e} | 46 | 86 | 147 | 271 |
| (C₁₂)₂MnCl₄ | major | 331 | 48 | 84 | 145 | 254 |
| | minor | 335 | 6 | 10 | 18 | 31 |
| | total | | 54 | 94 | 162 | 285 |
| (C₁₃)₂MnCl₄ | major | 331 | 52 | 87 | 158 | 264 |
| | minor | 343 | 8 | 13 | 22 | 37 |
| | total | | 60 | 100 | 180 | 301 |
| (C₁₄)₂MnCl₄ | major | 345 | 58 | 92 | 167 | 268 |
| | minor | 357 | 9 | 15 | 26 | 41 |
| | total | | 67 | 107 | 193 | 309 |
| (C₁₅)₂MnCl₄ | major | 340 | 63 | 96 | 184 | 282 |
| | minor | 362 | 10 | 16 | 28 | 44 |
| | total | | 73 | 112 | 213 | 325 |
| (C₁₆)₂MnCl₄ | major | 346 | 60 | 88 | 172 | 253 |
| | minor | 364 | 12 | 17 | 32 | 46 |
| | total | | 71 | 104 | 204 | 299 |

^{a}Cₙ = CₙH₂ₙ₊₁NH₃.
^{b}When a compound displays multiple transitions, the transition with the highest ΔS was labeled as a major transition.
^{c}The temperature of transition measured during the first heating scans are tabulated here.
^{d}This compound was synthesized and characterized for the completeness of the series.
^{e}Closely spaced transitions were not resolved.
^{f}These compounds was synthesized and characterized by DSC at a slow scan rate (0.5 K/min) to resolve major and minor transitions.
^{g}The difference between the previously reported value are within experimental uncertainties; however, the higher Tₜᵣ reported here may represent the higher purity of the sample, because some of the long-chain amines used in the previous literature have been shown to contain impurities, such as amines with different chain lengths (n ± 2), which typically give rise to lowering of Tₜᵣ (up to 4-5 K), ΔH and ΔS.

TABLE 3
Summary of previous reported chain melting transitions
in representative two-dimensional (CₙH₂ₙ₊₁NH₃)₂
CuX₄ perovskites (X = Cl, Br).

| | | | | | ΔS | ΔS |
|---|---|---|---|---|---|---|
| Chemical | | Tₜᵣ^{c} | ΔH | ΔH | (J mol⁻¹ | (J kg⁻¹ |
| Formula^{a} | Type^{b} | (K) | (kJ mol⁻¹) | (kJ kg⁻¹) | K⁻¹) | K⁻¹) |
| (C₈)₂CuCl₄ | major | 269 | 18 | 39 | 68 | 146 |
| | minor | 303 | 5 | 11 | 16 | 35 |
| | total | | 23 | 50 | 84 | 181 |
| (C₉)₂CuCl₄ | major | 294 | 23 | 46 | 78 | 158 |
| | minor | 303 | 5 | 9 | 15 | 31 |
| | total | total | 28 | 56 | 93 | 189 |
| (C₁₀)₂CuCl₄ | major | 309 | 35 | 67 | 113 | 216 |
| | minor | 312 | 5 | 9 | 15 | 28 |
| | total | | 39 | 75 | 128 | 244 |
| (C₁₁)₂CuCl₄ | major | 317^{e} | 36 | 65 | 112 | 204 |
| | minor | 327 | 6 | 11 | 18 | 33 |
| | total | | 42 | 76 | 130 | 237 |
| (C₁₂)₂CuCl₄ | major | 328 | 40 | 69 | 121 | 210 |
| | minor | 334 | 8 | 14 | 25 | 43 |
| | total | | 48 | 83 | 146 | 253 |
| (C₁₃)₂CuCl₄ | major | 333^{e} | 48 | 80 | 145 | 239 |
| | minor | 344 | 10 | 16 | 29 | 48 |
| | total | | 58 | 96 | 174 | 287 |
| (C₁₄)₂CuCl₄ | major | 334 | 50 | 61 | 150 | 237 |
| | minor | 356 | 11 | 83 | 31 | 48 |
| | total | | 61 | 144 | 181 | 285 |
| (C₁₅)₂CuCl₄ | major | 343 | 54 | 82 | 158 | 238 |
| | minor | 358 | 12 | 18 | 33 | 50 |
| | total | | 66 | 100 | 191 | 288 |
| (C₁₆)₂CuCl₄ | major | 345 | 36 | 52 | 103 | 150 |
| | minor | 354 | 8 | 12 | 23 | 33 |
| | minor | 360 | 14 | 20 | 39 | 56 |
| | total | | 58 | 83 | 165 | 239 |
| (C₉)₂CuBr₄ | | 303 | 16 | 24 | 52 | 78 |
| (C₁₁)₂CuBr₄ | major | 328 | 19 | 26 | 59 | 80 |
| | minor | 340 | 1 | 1 | 1 | 2 |
| | total | total | 20 | 27 | 60 | 82 |
| (C₁₂)₂CuBr₄ | | 337 | 22 | 29 | 64 | 85 |
| (C₁₃)₂CuBr₄ | | 344 | 30 | 38 | 86 | 109 |
| (C₁₄)₂CuBr₄ | | 348 | 28 | 34 | 80 | 98 |
| (C₁₅)₂CuBr₄ | | 354 | 39 | 46 | 110 | 131 |
| (C₁₆)₂CuBr₄ | major | 343 | 2 | 2 | 4 | 5 |
| | minor | 357 | 37 | 43 | 105 | 120 |
| | total | total | 39 | 45 | 109 | 125 |

^{a}Cₙ = CₙH₂ₙ₊₁NH₃.
^{b}When a compound displays multiple transitions, the transition with the highest ΔS was labeled as a major transition.
^{c}The temperature of transition measured during the first heating scans are tabulated here. These minor transitions were not resolved in the initial reports. Note that reported full DSC traces of (Cₙ)₂CuCl₄ (n = 2-14), are reported without integrated thermodynamic values.

TABLE 4
Summary of previous reported chain melting transitions
in representative two-dimensional (CₙH₂ₙ₊₁NH₃)₂CdCl₄ and (CₙH₂ₙ₊₁
NH₃)₂PbI₄ perovskites.

| | | | | | ΔS | ΔS |
|---|---|---|---|---|---|---|
| Chemical | | Tₜᵣ^{c} | ΔH | ΔH | (J mol⁻¹ | (J kg⁻¹ |
| Formula^{a} | Type^{b} | (K) | (kJ mol⁻¹) | (kJ kg⁻¹) | K⁻¹) | K⁻¹) |
| (C₇)₂CdCl₄ | major | 250 | 18 | 36 | 71 | 147 |
| | minor | 317 | 5 | 10 | 16 | 33 |
| | total | | 23 | 47 | 87 | 180 |
| (C₈)₂CdCl₄ | major | 269 | 15 | 28 | 54 | 105 |
| | minor | 308 | 5 | 10 | 17 | 32 |
| | total | | 20 | 38 | 71 | 138 |
| (C₁₀)₂CdCl₄ | minor | 308 | 8 | 14 | 25 | 44 |
| | major | 313 | 30 | 52 | 95 | 166 |
| | total | | 38 | 66 | 120 | 210 |
| (C₁₂)₂CdCl₄ | minor | 332 | 11 | 17 | 33 | 52 |
| | major | 334 | 44 | 69 | 130 | 208 |
| | total | | 54 | 87 | 163 | 260 |
| (C₁₄)₂CdCl₄ | major | 345 | 40 | 58 | 115 | 168 |
| | minor | 351 | 23 | 34 | 66 | 96 |
| | total | | 63 | 92 | 181 | 264 |
| (C₁₆)₂CdCl₄ | major | 345 | 40 | 55 | 117 | 158 |
| | minor | 352 | 8 | 10 | 22 | 30 |
| | minor | 356 | 32 | 43 | 88 | 120 |
| | | | 80 | 108 | 227 | 308 |
| (C₇)₂PbI₄ | minor | 271 | 7 | 7 | 25 | 26 |
| | major | 286 | 8 | 9 | 29 | 31 |
| | minor | 310 | 3 | 3 | 9 | 10 |
| | total | | 18 | 19 | 63 | 67 |
| (C₈)₂PbI₄ | minor | 252 | 15 | 15 | 58 | 59 |
| | major | 311 | 21 | 22 | 68 | 70 |
| | total | | 36 | 37 | 126 | 129 |
| (C₉)₂PbI₄ | minor | 252 | 7 | 7 | 28 | 28 |
| | major | 314 | 24 | 24 | 75 | 75 |
| | total | | 31 | 31 | 104 | 103 |
| (C₁₀)₂PbI₄ | minor | 259 | 10 | 10 | 39 | 38 |
| | minor | 284 | 8 | 8 | 30 | 29 |
| | major | 337 | 32 | 31 | 96 | 93 |
| | total | | 51 | 49 | 165 | 160 |
| (C₁₂)₂PbI₄ | minor | 315 | 11 | 10 | 35 | 32 |
| | major | 350 | 44 | 41 | 126 | 116 |
| | total | | 55 | 51 | 161 | 148 |
| (C₁₄)₂PbI₄ | minor | 329 | 11 | 9 | 32 | 28 |
| | major | 360 | 55 | 48 | 152 | 133 |
| | total | | 65 | 57 | 184 | 161 |
| (C₁₆)₂PbI₄ | minor | 340 | 12 | 10 | 34 | 28 |
| | major | 369 | 63 | 52 | 170 | 142 |
| | total | | 74 | 62 | 204 | 170 |

^{a}Cₙ = CₙH₂ₙ₊₁NH₃.
^{b}When a compound displays multiple transitions, the transition with the highest ΔS was labeled as a major transition.
^{c}The temperature of transition measured during the first heating scans are tabulated here. Note that 2-D Pb-I perovskites have partially interdigitated organic bilayers.

TABLE 5
Estimated barocaloric coefficients of representative two-
dimensional M—Cl perovskites (M = Mn and Cu). Note
that chain-melting phase transitions of these compounds
are also expected to be sensitive to pressure, with minor
transitions particularly more sensitive than major transitions.

| | | | | Estimated | ΔSₜᵣ | |
|---|---|---|---|---|---|---|
| Chemical | | Tₜᵣ | Δd | ΔVₜᵣ^{b} | (J K⁻¹ | dTₜᵣ/dP^{c} |
| Formula^{a} | Type | (K) | (Å) | (cm³ kg⁻¹) | kg⁻¹) | (K kbar⁻¹) |
| (C₁₂)₂MnCl₄ | major | 331 | 29.7 to | 61.7 | 254 | 24.3 |
| | | | 31.9; 2.2 | | | |
| | | | (7.4%) | | | |
| | minor | 335 | 31.9 to | 8.4 | 31 | 27.1 |
| | | | 32.2; 0.3 | | | |
| | | | (0.9%) | | | |
| (C₁₄)₂MnCl₄ | major | 345 | 33.3 to | 56.1 | 268 | 20.9 |
| | | | 35.5; 2.2 | | | |
| | | | (6.6%) | | | |
| | minor | 357 | 35.5 to | 23.0 | 41 | 56.0 |
| | | | 36.4; 0.9 | | | |
| | | | (2.5%) | | | |
| (C₁₀)₂CuCl₄ | major | 309 | 25.2 to | 57.1 | 216 | 26.4 |
| | | | 27.0; | | | |
| | | | 1.8 (7.1%) | | | |
| | minor | 312 | 27.0 to | 19.0 | 28 | 68.0 |
| | | | 27.6; | | | |
| | | | 0.6 (2.2%) | | | |
| (C₁₂)₂CuCl₄ | major | 328 | 28.8 to | 43.0 | 210 | 20.5 |
| | | | 30.3; 1.5 | | | |
| | | | (5.2%) | | | |
| | minor | 334 | 30.3 to | 22.9 | 43 | 53.3 |
| | | | 31.1; 0.8 | | | |
| | | | (2.6%) | | | |
| (C₁₄)₂CuCl₄ | major | 334 | 32.9 to | 41.8 | 237 | 17.6 |
| | | | 34.5; 1.6 | | | |
| | | | (4.9%) | | | |
| | minor | 356 | 34.5 to | 26.1 | 48 | 54.4 |
| | | | 35.5; 1.0 | | | |
| | | | (2.9%) | | | |

^{a}Cₙ = CₙH₂ₙ₊₁NH₃.
^{b}Note that the specific volume of each phase was estimated using the relationship, V = dA_{c} × M_{w}/N_{A}, where A_{c} is the area of metal-halide sheet per chain, d is the interlayer distance, M_{w} is the molecular weight, and N_{A} is Avogadro's number. We used this estimation because the unit cell parameters of intermediate phases were often not available. The reported mean values of A_{c} are 26.5 Å² and 27.5 Å² for Mn—Cl and Cu—Cl perovskites, respectively. Note that this approach is likely to overestimate the volume change.
^{c}Barocaloric coefficients were calculated through the Clausius-Clapeyron equation (dTₜᵣ/dP = ΔVₜᵣ/ΔSₜᵣ).

TABLE 6
The linear relationship between chain length and total change in
entropy (ΔSₜₒₜₐₗ) in chain-melting transition of 2-D perovskites.

| | | | | | | Restriction | Area per |
|---|---|---|---|---|---|---|---|
| | Chain | | y- | | Flexibility | parameter | chain |
| Compound^{a} | length | Slope | intercept | R² | number Φ | β | (Å²) |
| (Cₙ)₂MnCl₄ | odd | 19.4 | −75.3 | 0.995 | 3.2 | 2.5 | 26.5 |
| | even | 18.5 | −63.7 | 0.994 | 3.0 | 2.1 | |
| (Cₙ)₂CdCl₄ | even | 18.7 | −71.4 | 0.976 | 3.1 | 2.4 | 27.7 |
| (Cₙ)₂CuCl₄ | odd | 16.9 | −55.8 | 0.960 | 2.8 | 2.0 | 27.5 |
| | even | 15.5 | −35.2 | 0.965 | 2.5 | 1.0 | |
| (Cₙ)₂CuBr₄ | odd | 12.5 | −77.2 | 0.999 | 2.1 | 5.1 | 30.3 |
| | even | 11.3 | −73.2 | 0.946 | 2.0 | 5.5 | |

^{a}Cₙ = CₙH₂ₙ₊₁NH₃.

TABLE 7
Summary of characteristic infrared signals of (DA)₂MnCl₄ and (NA)₂CuBr₄.

| | Mode^{a} | (DA)₂MnCl₄ | (DA)₂MnCl₄ | (NA)₂CuBr₄ | (NA)₂CuBr₄ |
|---|---|---|---|---|---|
| Structure | (cm⁻¹) | LT band (cm⁻¹) | HT band (cm⁻¹) | LT band (cm⁻¹) | HT band (cm⁻¹) |
| Alkyl chain | vₐₛ(C—H) | 2919 | 2919 | 2919 | 2920 |
| (CₙH₂ₙ₊₁NH₃⁺) | vₛ(C—H) | 2850 | 2852 | 2851 | 2853 |
| | v(CH₂)_{rocking} | 727-720 | 721 | 722 | 722 |
| | | doublet | singlet | singlet | singlet |
| | v(CH₂)_{bending} | 1472-1463 | 1464 | 1467 | 1468 |
| | | doublet | singlet | shoulders | shoulders |
| | | | | (~1440, ~1455) | (~1440, ~1455) |
| | v(CH₂)_{wagging} | | band progression | 1306 | 1306 |
| | two units, | | ~1310 | | (broadening) |
| | in-plane | | (shoulder, contribute | | 1312 |
| | ~1310 (kink) | | to broadening) | | (new peak) |
| | v(CH₂)_{wagging} | | band progression | 1360 | not detected |
| | two units, | | ~1367 (shoulder) | | |
| | out-of-plane | | | | |
| | ~1360 (kink) | | | | |
| | v(CH₂)_{wagging} | not detected | not detected | not detected | not detected |
| | single unit | | | | |
| | ~1350 (gg) | | | | |
| | (CH₂)_{wagging} | not detected | not detected | 1340 | not detected |
| | single unit | | | | |
| | ~1340 (tg, | | | | |
| | chain end) | | | | |
| | vₛ(CH₃)_{bending} | 1375 | 1379 | 1378 | 1379 |
| | ~1376 | | | | |
| Polar head | vₛ(NH₃)_{bending} | 1496 | 1496 | 1480 | 1480 |
| (CₙH₂ₙ₊₁NH₃⁺) | | narrow | broad | | |
| | vₐₛ(NH₃)_{bending} | 1585 | 1579 | 1569-1572 | 1569 |
| | | narrow | broad | doublet | singlet |

^{a}vₛ and vₐₛ refer to symmetric and anti-symmetric modes, respectively.
^{b}previous reports on Cd—Cl and Mn—Cl analogs revealed that the peak near 1337 cm⁻¹ does not depend on chain conformation (20, 37).

TABLE 8
Summary of characteristic infrared signals of representative 2-D perovskites.

| | Mode^{a} | | | | | |
|---|---|---|---|---|---|---|
| Structure | (cm⁻¹) | (C₁₀)₂CdCl₄ | (C₉)₂CuCl₄ | (C₁₀)₂CuCl₄ | (C₁₂)₂CuCl₄ | (C₁₄)₂MnCl₄ |
| Alkyl chain | vₐₛ(C—H) | not | not | not | 2919 → | not |
| (CₙH₂ₙ₊₁NH₃⁺) | ~2920 | discussed | discussed | discussed | 2923 | discussed |
| | | | | | increase | |
| | vₛ(C—H) | 2851 → | 2852 → | not | 2850 → | not |
| | ~2850 | 2853 | 2854 | discussed | 2852 | discussed |
| | | increase | increase | | increase | |
| | v(CH₂)_{rocking} | 728-720 | 727-722 | 730-725 | 730-725 | 729-719 |
| | | doublet to | doublet to | doublet to | doublet to | doublet to |
| | | singlet | singlet | singlet | singlet | singlet |
| | v(CH₂)_{bending} | ~1460 | 1471-1466 | 1472-1467 | 1472-1467 | 1475-1463 |
| | | doublet to | doublet to | doublet to | doublet to | doublet to |
| | | singlet | singlet | singlet | singlet | singlet |
| | v(CH₂)_{wagging} | 1306 | 1310 | 1308 | 1310 | 1305 |
| | two units, in- | broad | broad | broad | broad | broad |
| | plane | | | | | |
| | ~1310 (kink^{b}) | | | | | |
| | v(CH₂)_{wagging} | not | 1364 | | 1367 | 1367 |
| | two units, | detected | shoulder | | shoulder | shoulder |
| | out-of-plane | | | | | |
| | ~1360 (kink^{b}) | | | | | |
| | v(CH₂)_{wagging} | not | not | not | not | not |
| | single unit | detected | detected | detected | detected | detected |
| | ~1350 (gg) | | | | | |
| | (CH₂)_{wagging} | not | 1340 | 1341 | 1342 | not |
| | single unit | detected | | | | detected |
| | ~1340 (tg, | | | | | |
| | chain end) | | | | | |
| | vₛ(CH₃)_{bending} | 1376^{a} | not | 1376 → | 1377 → | 1375 → |
| | ~1376 | | discussed | 1378 | 1379 | 1379 |
| | | | | increase | increase | increase |
| Polar head | vₛ(NH₃)_{bending} | ~1488 | 1491-1479 | 1492-1480 | 1492-1480 | ~1496 |
| (CₙH₂ₙ₊₁NH₃_{⁺}) | | doublet to | doublet to | doublet to | doublet to | doublet to |
| | | singlet^{c} | singlet | singlet | singlet | singlet^{c} |
| | vₐₛ(NH₃)_{bending} | 1589^{d} | 1583→ | 1583→ | 1584→ | not |
| | | | 1575 | 1576 | 1575 | discussed |
| | | | decrease | decrease | decrease | |
| Ref. | | (20, 30) | (34) | (19) | (33) | (37) |

^{a}vₛ and vₐₛ refer to symmetric and anti-symmetric modes, respectively.
^{b}kink generally refers to gt₂ₙ₊₁9′-type conformational defects.
^{c}the splitting in Mn—Cl and Mn—Cl analogs tends to be small and often not discernible.
^{d}temperature-dependent band progression is not discussed

TABLE 9
Comparison of predicted and experimentally determined barocaloric coefficients (dTₜᵣ/dP).
Note that the barocaloric coefficient were estimated using the Clausius-Clapeyron equation
dTₜᵣ/dP = ΔVₜᵣ/ΔSₜᵣ, with ΔVₜᵣ determined through powder X-ray diffraction or
Helium pycnometry, and ΔSₜᵣ measured at ambient pressure. ΔSₜᵣ values for (DA)₂MnCl₄
and (NA)₂CuBr₄ were 230 and 78 J K⁻¹ kg⁻¹, respectively.

| | PXRD^{a} | | He pycnometry | | | | | HP-DSC | |
|---|---|---|---|---|---|---|---|---|---|
| | ΔV | | | ΔV | | dTₜᵣ/dP | | dTₜᵣ/dP | |
| | (cm³ | dTₜᵣ/dP | | (cm³ kg⁻¹) | | (K kbar⁻¹) | | (K kbar⁻¹) | |
| Compound | kg⁻¹) | (K kbar⁻¹) | | heating | cooling | heating | cooling | heating | cooling |
| (DA)₂MnCl₄ | 65.07 | 28.3 | linear | 54.7 | 53.06 | 23.8 | 23.1 | 22.1 ± 0.7 | 20.6 ± 0.8 |
| | (7.95%) | | fit | (6.60%) | (6.38%) | | | | |
| | | | nearest | 49.3 ± 3.4 | 47.5 ± 5.8 | 21.4 ± 1.5 | 20.7 ± 2.5 | | |
| | | | points | (5.9%) | (5.7%) | | | | |
| (NA)₂CuBr₄ | 24.93 | 32 | linear | 19.32 | 19.25 | 24.8 | 24.7 | 26.9 ± 0.4 | 26.5 ± 0.5 |
| | (3.95%) | | fit | (3.1%) | (3.1%) | | | | |
| | | | nearest | 20.0 ± 1.9 | 19.7 ± 2.4 | 25.6 ± 2.4 | 25.3 ± 3.0 | | |
| | | | points | (3.2%) | (3.1%) | | | | |

^{a}PXRD data was obtained during cooling and the volume change was obtained through linear fit away from the transition.

TABLE 10
Selected geometric parameters of
(DA)₂MnCl₄ at 100 K, 270 K, and 330 K.

| Temperature (K) | 100 K | | 270 K | | 330 K | |
|---|---|---|---|---|---|---|
| Space Group | P1̅ | | C2/m | | Cccm | |
| V (Å³) | 672.07 | (14) | 1396.0 | (2) | 3069.5 | (9) |
| Nearest Mn—Mn (Å) | 5.0548 | (6) | 5.1252 | (3) | 5.1619 | (3) |
| Nearest N—N (Å) | 5.0548 | (6) | 4.9369 | (3) | 5.0726 | (3) |
| Interlayer distance | 26.419 | (3) | 26.685 | (2) | 28.800 | (1) |
| (Å) | | | | | | |
| Distance between N | 2.325 | (4) | 2.305 | (1) | 2.262 | (1) |
| atoms and [MnCl₄]²⁻ (Å)^{a} | | | | | | |
| Chain tilt angle relative to | 42.8 (10) | | 41.7 | (1) | 25.8 | (1) |
| [MnCl₄]²⁻ sheet normal | [42.8 (10)] | | | | | |
| (°) | | | | | | |
| best line (C1 to C10) | | | | | | |
| [Disordered position] | | | | | | |
| Chain tilt angle relative to | 65.1 (10) | | 70.9 | (1) | 69.5 | (1) |
| [MnCl₄]²⁻ sheet normal | [65.4 (10)] | | | | | |
| (°) | | | | | | |
| mean plane (C2 to C10) | | | | | | |
| [Disordered position] | | | | | | |
| Cross-sectional area per | 25.439 | (6) | 26.157 | (4) | 26.635 | (8) |
| chain (Å²)^{b} | | | | | | |
| Cross-sectional area per | 25.551 | (4) | 26.268 | (2) | 26.645 | (2) |
| chain (Å²)^{c} | | | | | | |

^{a}calculated as the distance between mean plane of four N atoms and mean plane of [MnCl₄]²⁻ layer
^{b}calculated from V/(Zd), where V is the unit cell volume, Z is the number of molecules in the unit cell, and d is the interlayer distance.
^{c}estimated by d², where d is the nearest metal-metal distance.

TABLE 11
Selected geometric parameters of
(NA)₂CuBr₄ at 100 K, 270 K, and 335 K.

| Temperature (K) | 100 K | | 270 K | | 335 K | |
|---|---|---|---|---|---|---|
| Space Group | P1̅ | | P1̅ | | Cmca | |
| V (Å³) | 1330.3 | (2) | 1382.70 | (7) | 2966.0 | (3) |
| Nearest Cu—Cu (Å) | 5.4102 | (4) | 5.5173 | (1) | 5.4927 | (3) |
| Nearest N—N (Å) | 5.117 | (7) | 5.1667 | (1) | 5.4927 | (3) |
| Interlayer distance | 23.054 | (2) | 23.254 | (1) | 24.578 | (2) |
| (Å) | | | | | | |
| Distance between N | 2.201 | (4) | 2.150 | (1) | 2.138 | (1) |
| atoms and [MnCl₄]²⁻ (Å)^{a} | | | | | | |
| Chain tilt angle relative to | 46.9 | (2) | 46.67 (1) | | 35.4 | (1) |
| [CuBr₄]²⁻ sheet normal | | | [43.37 (1)] | | | |
| (°) | | | | | | |
| best line (C1 to C9) | | | | | | |
| [Disordered position] | | | | | | |
| Chain tilt angle relative to | 48.7 | (2) | 48.49 (1) | | | |
| [CuBr₄]²⁻ sheet normal | | | [47.00 | (1)] | | |
| (°) | | | | | | |
| best line (C11 to C19) | | | | | | |
| [Disordered position] | | | | | | |
| Chain tilt angle relative to | 58.9 | (3) | 64.60 (1) | | 68.4 | (1) |
| [CuBr₄]²⁻ sheet normal | | | [72.16 (1)] | | | |
| (°) | | | | | | |
| mean plane (C2 to C9) | | | | | | |
| [Disordered position] | | | | | | |
| Chain tilt angle relative to | 54.8 | (3) | 52.36 (1) | | | |
| [CuBr₄]²⁻ sheet normal | | | [54.72 (1)] | | | |
| (°) | | | | | | |
| mean plane (C12 to C19) | | | | | | |
| [Disordered position] | | | | | | |
| Cross-sectional area per | 28.852 | (5) | 29.730 | (2) | 30.169 | (4) |
| chain (Å²)^{b} | | | | | | |
| Cross-sectional area per | 29.270 | (3) | 30.440 | (7) | 30.169 | (2) |
| chain (Å²)^{c} | | | | | | |

^{a}calculated as the distance between mean plane of four N atoms and mean plane of [MnCl₄]²⁻ layer
^{b}calculated from V/(Zd), where V is the unit cell volume, Z is the number of molecules in the unit cell, and d is the interlayer distance.
^{c}calculated by d₁ × d₂, where di and d2 are metal-metal distances.

TABLE 12
Donor-acceptor (N•••Cl) distances and bond angles
for LT and HT phases in (DA)₂MnCl₄ and (NA)₂CuBr₄.

| | | | | | | | Tilt of |
|---|---|---|---|---|---|---|---|
| | | | | | | | NH₃ |
| | | | H•••A | D•••A | ∠(DHA) | | group |
| Compound | T (K) | N—H•••X | (Å) | (Å) | (°) | Occupancy | (°)^{a} |
| (DA)₂MnCl₄ | 270 | Equatorial | 2.4213(34) | 3.291(37) | 166.12(44) | | 61.4 |
| | | Axial 1 | 2.4668(14) | 3.3310(75) | 164.12(42) | | (16) |
| | | Axial 2 | 2.3544(15) | 3.226(66) | 165.2(26) | | |
| | 330 | Equatorial | 2.6051(17) | 3.384(20) | 146.53(74) | | 67.66 |
| | | Axial 1 | 2.7753(4) | 3.622(29) | 158.8(17) | | (92) |
| | | Axial 2 | 2.7215(22) | 3.3332(84) | 127.02(52) | | |
| (NA)₂CuBr₄ | 270 | Equatorial | 2.7385(5) | 3.5262(45) | 148.22(31) | Chain A | 50.682 |
| | | Axial 1 | 3.0803(7) | 3.9226(54) | 158.73(35) | Part 1 (0.35) | (60) |
| | | Axial 2 | 2.7546(6) | 3.4350(48) | 134.20(31) | | |
| | | Equatorial | 2.5836(6) | 3.431(5) | 159.52(31) | Chain A | 57.37 |
| | | Axial 1 | 2.6022(6) | 3.4350(48) | 156.18(31) | Part 2 (0.65) | (39) |
| | | Axial 2 | 2.9046(7) | 3.7516(55) | 159.55(35) | | |
| | | Equatorial | 2.6289(6) | 3.4424(55) | 152.25(31) | Chain B | 54.76 |
| | | Axial 1 | 2.5648(6) | 3.3696(49) | 150.76(31) | Part 1 (0.53) | (41) |
| | | Axial 2 | 2.7882(7) | 3.6367(55) | 159.92(35) | | |
| | | Equatorial | 2.6929(6) | 3.5496(50) | 161.86(31) | Chain B | 81.09 |
| | | Axial 1 | 2.7716(6) | 3.3696(49) | 125.71(31) | Part 2 (0.47) | (88) |
| | | Axial 2 | 3.3243(7) | 4.0423(54) | 139.38(35) | | |
| | 335 | Equatorial | 2.6419(12) | 3.506(21) | 162.449(3) | | 70.7 |
| | | Axial 1 | 3.1158(5) | 3.9023(25) | 147.952(4) | | (15) |
| | | Axial 2 | 3.1905(10) | 3.8858(25) | 136.106(1) | | |

^{a}the tilt angle is defined as the angle between a line connecting the atoms N and C and a plane through the metal atoms of the inorganic layers.

TABLE 13
Tabulation of dihedral angle φ of alkylammonium chains at 100K, 270K, and 330K for (DA)₂MnCl₄.

| | Atoms | φ (°) | Occupancy | Atoms | φ (°) | Occupancy |
|---|---|---|---|---|---|---|
| T (K) | Part 1^{a} | Part 1 | Part 1 | Part 2^{a} | Part 2 | Part 2 |
| 100 | N1-C1-C2-C3 | 179.6 (10) | 0.504(13) | N1A-C1A-C2A-C3A | 178.2 (10) | 0.496(13) |
| | C1-C2-C3-C4 | −68 (2) | 0.504(13) | C1A-C2A-C3A-C4A | 70 (2) | 0.496(13) |
| | C2-C3-C4-C5 | −178.2 (13) | 0.504(13) | C2A-C3A-C4A-C5A | 177.5 (14) | 0.496(13) |
| | C3-C4-C5-C6 | 178.0 (14) | 0.504(13) | C3A-C4A-C5A-C6A | −178.4 (15) | 0.496(13) |
| | C4-C5-C6-C7 | −179.7 (15) | 0.504(13) | C4A-C5A-C6A-C7A | −179.3 (16) | 0.496(13) |
| | C5-C6-C7-C8 | 178.6 (14) | 0.504(13) | C5A-C6A-C7A-C8A | −179.3 (15) | 0.496(13) |
| | C6-C7-C8-C9 | 178.4 (15) | 0.504(13) | C6A-C7A-C8A-C9A | 177.7 (16) | 0.496(13) |
| | C7-C8-C9-C10 | 175.9 (17) | 0.504(13) | C7A-C8A-C9A-C10A | 179.3 (17) | 0.496(13) |
| 270 | N1-C1-C2-C3 | 174 (3) | | | | |
| | C1-C2-C3-C4 | −65 (5) | | | | |
| | C2-C3-C4-C5 | 179 (3) | | | | |
| | C3-C4-C5-C6 | 177 (4) | | | | |
| | C4-C5-C6-C7 | −175 (5) | | | | |
| | C5-C6-C7-C8 | 180 (5) | | | | |
| | C6-C7-C8-C9 | −174 (5) | | | | |
| | C7-C8-C9-C10 | −169 (8) | | | | |
| 330 | N1-C1-C2-C3 | 174 (3) | | | | |
| | C1-C2-C3-C4 | −166 (3) | | | | |
| | C2-C3-C4-C5 | −180 (3) | | | | |
| | C3-C4-C5-C6 | 163 (4) | | | | |
| | C4-C5-C6-C7 | 154 (4) | | | | |
| | C5-C6-C7-C8 | 160 (4) | | | | |
| | C6-C7-C8-C9 | 165 (5) | | | | |
| | C7-C8-C9-C10 | −151 (5) | | | | |

^{a}Part 1 and part 2 refer to the sets of disordered positions of the alkylammonium chains conformer B (C2-C3 gauche bond)

TABLE 14
Tabulation of dihedral angle φ of alkylammonium chains at 100K, 270K, and 335K for (NA)₂CuBr₄.

| | Atoms | φ (°) | Occupancy | Atoms | φ (°) | Occupancy |
|---|---|---|---|---|---|---|
| T (K) | Part 1^{a} | Part 1 | Part 1 | Part 2^{a} | Part 2 | Part 2 |
| 100 | N1-C1-C2-C3 | −175.0 (6) | | | | |
| | C1-C2-C3-C4 | −65.7 (9) | | | | |
| | C2-C3-C4-C5 | −177.4 (7) | | | | |
| | C3-C4-C5-C6 | 178.8 (7) | | | | |
| | C4-C5-C6-C7 | 179.9 (6) | | | | |
| | C5-C6-C7-C8 | 179.0 (7) | | | | |
| | C6-C7-C8-C9 | 177.9 (7) | | | | |
| | N2-C11-C12-C13 | 70.0 (7) | | | | |
| | C11-C12-C13-C14 | −177.6 (6) | | | | |
| | C12-C13-C14-C15 | 174.1 (6) | | | | |
| | C13-C14-C15-C16 | 178.6 (6) | | | | |
| | C14-C15-C16-C17 | −179.5 (6) | | | | |
| | C15-C16-C17-C18 | −175.1 (6) | | | | |
| | C16-C17-C18-C19 | −177.3 (7) | | | | |
| 270 | N1-C1-C2-C3 | −173.0 (17) | 0.528(9) | N1A-C1A-C2A-C3A | 172 (2) | 0.472(9) |
| | C1-C2-C3-C4 | −39 (3) | 0.528(9) | C1A-C2A-C3A-C4A | −178 (3) | 0.472(9) |
| | C2-C3-C4-C5 | −176 (3) | 0.528(9) | C2A-C3A-C4A-C5A | 174 (3) | 0.472(9) |
| | C3-C4-C5-C6 | 178 (3) | 0.528(9) | C3A-C4A-C5A-C6A | 180 (4) | 0.472(9) |
| | C4-C5-C6-C7 | 178 (4) | 0.528(9) | C4A-C5A-C6A-C7A | 173 (4) | 0.472(9) |
| | C5-C6-C7-C8 | 168 (4) | 0.528(9) | C5A-C6A-C7A-C8A | −174 (4) | 0.472(9) |
| | C6-C7-C8-C9 | 164 (4) | 0.528(9) | C6A-C7A-C8A-C9A | −164 (4) | 0.472(9) |
| | N2-C11-C12-C13 | −67 (5) | 0.350(15) | N2A-C11A-C12A-C13A | 77 (2) | 0.650(15) |
| | C11-C12-C13-C14 | 173 (4) | 0.350(15) | C11A-C12A-C13A-C14A | −176.9 (19) | 0.650(15) |
| | C12-C13-C14-C15 | −159 (5) | 0.350(15) | C12A-C13A-C14A-C15A | 169 (2) | 0.650(15) |
| | C13-C14-C15-C16 | −175 (5) | 0.350(15) | C13A-C14A-C15A-C16A | −180 (2) | 0.650(15) |
| | C14-C15-C16-C17 | −173 (6) | 0.350(15) | C14A-C15A-C16A-C17A | 175 (3) | 0.650(15) |
| | C15-C16-C17-C18 | −177 (6) | 0.350(15) | C15A-C16A-C17A-C18A | 176 (3) | 0.650(15) |
| | C16-C17-C18-C19 | 159 (6) | 0.350(15) | C16A-C17A-C18A-C19A | −170 (3) | 0.650(15) |
| 335 | N1-C1-C2-C3 | 156 (3) | | | | |
| | C1-C2-C3-C4 | 44 (7) | | | | |
| | C2-C3-C4-C5 | 156 (4) | | | | |
| | C3-C4-C5-C6 | −128 (8) | | | | |
| | C4-C5-C6-C7 | 160 (7) | | | | |
| | C5-C6-C7-C8 | 174 (6) | | | | |
| | C6-C7-C8-C9 | −142 (10) | | | | |

^{a}Part 1 and part 2 refer to the sets of disordered positions of the alkylammonium chains conformer conformers A (C1-C2 gauche) and B (C2-C3 gauche bond)

TABLE 15
Crystallographic data for (DA)₂MnCl₄ collected at 100 K, 270 K, and 330 K.

| | (DA)₂MnCl₄ | | (DA)₂MnCl₄ | | (DA)₂MnCl₄ | |
|---|---|---|---|---|---|---|
| Formula | C₂₀H₄₈Cl₄MnN₂ | | C₂₀H₄₈Cl₄MnN₂ | | C₂₀H₄₈Cl₄MnN₂ | |
| Temperature (K) | 100 | (2) | 270 | (2) | 330 | (2) |
| Crystal System | Triclinic | | Monoclinic | | Orthorhombic | |
| Space Group | P1̅ | | C2/m | | Cccm | |
| a (Å) | 5.0548 | (6) | 7.1857 | (6) | 7.3264 | (5) |
| b (Å) | 5.0626 | (6) | 7.3100 | (6) | 57.601 | (15) |
| c (Å) | 26.419 | (3) | 26.685 | (2) | 7.2735 | (6) |
| α (°) | 95.516 | (4) | 90 | | 90 | |
| β (°) | 92.683 | (4) | 95.150 | (1) | 90 | |
| γ (°) | 90.904 | (4) | 90 | | 90 | |
| V (Å³) | 672.07 | (14) | 1396.0 | (2) | 3069.5 | (9) |

| Z | 1 | 2 | 4 |
|---|---|---|---|
| Radiation, λ (Å) | MoKα, 0.71073 | MoKα, 0.71073 | MoKα, 0.71073 |
| μ (mm⁻¹) | 0.90 | 0.86 | 0.79 |
| Crystal Size (mm) | 0.18 × 0.12 × 0.08 | 0.18 × 0.12 × 0.08 | 0.18 × 0.12 × 0.08 |
| Max. and min. | 0.767 and 0.635 | 0.767 and 0.492 | 0.801 and 0.478 |
| transmission | | | |
| Completeness to 2Θ | 98.6% | 98.5% | 97.1% |
| | (2Θ = 25.123°) | (2Θ = 25.191°) | (2Θ = 25.607°) |
| No. of measured, | 8898, 2353, 1872 | 11186, 1345, 1062 | 18017, 1533, 928 |
| independent and | | | |
| observed [I > 2σ(I)] | | | |
| reflections | | | |
| Rᵢₙₜ | 0.055 | 0.061 | 0.100 |
| (sin □/λ)ₘₐₓ (Å⁻¹) | 0.597 | 0.599 | 0.608 |
| Data/Restraints/Parameters | 2353/134/238 | 1345/0/124 | 1533/170/116 |
| Goodness of Fit on F² | 1.10 | 1.05 | 1.08 |
| R^{a}, wR₂^{b} | 0.066, 0.171 | 0.061, 0.181 | 0.097, 0.259 |
| [I > 2σ(I)] | | | |
| Largest Diff. Peak | 1.32 and −0.65 | 1.64 and −0.77 | 0.78 and −0.41 |
| and Hole (e Å⁻³) | | | |

^{a}R₁ = Σ||Fₒ| − |F_{c}||/Σ|Fₒ|.
^{b}wR₂ = {Σ[w(Fₒ² − F_{c}²)²]/Σ[w(Fₒ²)²]}^{1/2}.

TABLE 16
Crystallographic data for (NA)₂CuBr₄ collected at 100 K, 270 K, and 335 K.

| | (NA)₂CuBr₄ | | (NA)₂CuBr₄ | | (NA)₂CuBr₄ | |
|---|---|---|---|---|---|---|
| Formula | C₁₈H₄₄Br₄CuN₂ | | C₁₈H₄₄Br₄CuN₂ | | C₁₈H₄₄Br₄CuN₂ | |
| Temperature (K) | 100 | (2) | 270 | (2) | 335 | (2) |
| Crystal System | Triclinic | | Triclinic | | Orthorhombic | |
| Space Group | P1̅ | | P1̅ | | Cmca | |
| a (Å) | 7.4107 | (7) | 7.6549 | (2) | 49.155 | (3) |
| b (Å) | 7.9092 | (8) | 7.9674 | (2) | 7.7844 | (5) |
| c (Å) | 23.054 | (2) | 23.2541 | (7) | 7.7512 | (5) |
| α (°) | 82.835 | (2) | 80.4332 | (9) | 90 | |
| β (°) | 82.901 | (3) | 81.4664 | (9) | 90 | |
| γ (°) | 89.808 | (2) | 89.8532 | (8) | 90 | |
| V (Å³) | 1330.3 | (2) | 1382.70 | (7) | 2966.0 | (3) |

| Z | 2 | 2 | 4 |
|---|---|---|---|
| Radiation, λ (Å) | MoKα, 0.71073 | MoKα, 0.71073 | MoKα, 0.71073 |
| μ (mm⁻¹) | 6.83 | 6.58 | 6.13 |
| Crystal Size (mm) | 0.24 × 0.12 × 0.06 | 0.24 × 0.12 × 0.06 | 0.24 × 0.12 × 0.06 |
| Max. and min. | 0.646 and 0.428 | 0.801 and 0.658 | 0.694 and 0.290 |
| transmission | | | |
| Completeness to 2Θ | 99.3% | 99.4% | 96.8% |
| | (2Θ = 25.092°) | (2Θ = 25.026°) | (2Θ = 25.004°) |
| No. of measured, | | | |
| independent and | 20830, 4693, 3569 | 15136, 4857, 3313 | 17040, 1291, 790 |
| observed [I > 2σ(I)] | | | |
| reflections | | | |
| Rᵢₙₜ | 0.065 | 0.042 | 0.112 |
| (sin q/λ)ₘₐₓ (Å⁻¹) | 0.597 | 0.595 | 0.595 |
| Data/Restraints/Parameters | 4693/0/233 | 4857/988/399 | 1291/151/103 |
| Goodness of Fit on F² | 1.05 | 1.03 | 1.09 |
| R^{a}, wR₂^{b} | 0.040, 0.104 | 0.042, 0.099 | 0.104, 0.261 |
| [I > 2σ(I)] | | | |
| Largest Diff. Peak | 1.21 and −0.78 | 0.58 and −0.61 | 0.46 and −0.47 |
| and Hole (e Å⁻³) | | | |

^{a}R₁ = Σ||Fₒ| − |F_{c}||/Σ|Fₒ|.
^{b}wR₂ = {Σ[w(Fₒ² − F_{c}²)²]/Σ[w(Fₒ²)²]}^{1/2}.

TABLE 17
Comparison of crystallographic unit cell data for ordered phase of (DA)₂MnCl₄.

| | (DA)₂MnCl₄ | | (DA)₂MnCl₄ | | (DA)₂MnCl₄ | |
|---|---|---|---|---|---|---|
| | experimental | | experimental | | reported (1) | |
| Formula | C₂₀H₄₈Cl₄MnN₂ | | C₂₀H₄₈Cl₄MnN₂ | | C₂₀H₄₈Cl₄MnN₂ | |
| Temperature (K) | 100 | (2) | 270 | (2) | 298 | (3) |
| Crystal system | Triclinic | | Monoclinic | | Monoclinic | |
| Space Group | P1̅ | | C2/m | | P2₁/a | |
| a (Å) | 5.0548 | (6) | 7.1857 | (6) | 7.213 | (8) |
| b (Å) | 5.0626 | (6) | 7.3100 | (6) | 7.337 | (2) |
| c (Å) | 26.419 | (3) | 26.685 | (2) | 26.747 | (21) |
| α (°) | 95.516 | (4) | 90 | | 90 | |
| β (°) | 92.683 | (4) | 95.150 | (1) | 94.64 | (5) |
| γ (°) | 90.904 | (4) | 90 | | 90 | |
| V (Å³) | 672.07 | (14) | 1396.0 | (2) | 1411 | (2) |

| Z | 1 | 2 | 2 |
|---|---|---|---|
| Radiation, λ (Å) | MoKα, 0.71073 | MoKα, 0.71073 | CuKα, 1.5418 |
| No. of measured, | 8898, 2353, 1872 | 11186, 1345, 1062 | No. of independent |
| independent and | | | reflections 1393 |
| observed [I > 2σ(I)] | | | |
| reflections | | | |
| Rᵢₙₜ | 0.055 | 0.061 | 0.086 (for 1204 |
| | | | reflections) |
| (sin q/λ)ₘₐₓ (Å⁻¹) | 0.597 | 0.599 | |
| R[F² > 2σ(F²)], | 0.066, 0.171, 1.10 | 0.061, 0.181, 1.05 | |
| wR(F²), S | | | |
| Data/Restraints/Parameters | 2353/134/238 | 1345/0/124 | |
| Largest Diff. Peak | 1.32 and −0.65 | 1.64 and −0.77 | |
| and Hole (e Å⁻³) | | | |

TABLE 18
Phase-change properties and barocaloric effects of representative barocaloric materials.

| | | | ΔSₜᵣ^{c} | dTₜᵣ/dP | dTₜᵣ/dP | | | ΔS_{it, rev}^{f} | | |
|---|---|---|---|---|---|---|---|---|---|---|
| | Chemical | | (J kg⁻¹ | heating | cooling | ΔT_{hys}^{d} | Pᵣₑᵥ^{e} | (J kg⁻¹ | ΔP^{f} | |
| Type | Formula^{a} | Tₜᵣ^{b} | K⁻¹) | (K kbar⁻¹) | (K kbar⁻¹) | (K) | (bar) | K⁻¹) | (bar) | Ref |
| 2-D perovskite | (DA)₂MnCl₄ | 310 | 230 | 22.1 | 20.6 | 1.4 | 66 | 75 | 150 | This |
| | (NA)₂CuBr₄ | 303 | 78 | 26.9 | 26.5 | 0.4 | 16 | 68 | 150 | work |
| 3-D hybrid | [(CH₃)₄N][Mn(N₃)₃] | 305 | 80 | 12^{g} | | 7 | 583 | 70 | 900 | (59) |
| perovskite | [TPrA][Mn(dca)₃] | 330 | 43 | 23.1^{g} | | 0.9 | 39 | 31 | 70 | (60) |
| | [TPrA][Cd(dca)₃] | 386 | 16 | 38.2^{g} | | 1.4 | 37 | 11.5 | 70 | (61) |
| Organic | (CH₃)₂C(CH₂OH)₂ | 314 | 389 | 11.3 | 9.3 | 14 | 1505 | 445 | 2500 | (62, 63) |
| plastic | (CH₃)C(CH₂OH)₃ | 354 | 485 | 7.9 | 9.4 | 3.7 | 394 | 490 | 2400 | (64) |
| crystal | (CH₃)₃C(CH₂OH) | 232 | 204 | 22 | 11.9 | 20.3 | 1706 | 290 | 2600 | (64) |
| | C₆₀ | 259 | 27 | 16.7 | 17.2 | 3 | 174 | 32 (42) | 1000 (4100) | (65) |
| Inorganic | (NH₄)₂SO₄ | 222 | 65 | −5.7 | −4.5 | 1 | 175 | 60 | 1000 | (66) |
| | AgI | 420 | 64 | −14 | −12.8 | 25 | 1786 | 60 | 1000 | (67) |
| | Fe₄₉Rh₅₁ | 310 | 13 | 5.4 | 6.4 | 10 | 1563 | 13 | 2500 | (68) |
| | Ni_{0.85}Fe_{0.15}S^{h} | 303 | 53 | −7.5 | | 11.5 | 1533 | | | (69) |
| Spin | Fe₃(bntrz)₆(tcnset)₆ | 318 | 80 | 25 | 25 | 2 | 80 | 80 (120) | 550 (2600) | (70) |
| crossover | [FeL₂](BF₄)₂^{h, i} | 262 | 86 | 10^{j} | 10^{j} | 4 | 400 | | | (71) |
| complex | | | | | | | | | | |

^{a}DA = decylammonium; NA = nonylammonium; TPrA = tetrapropylammonium; bntrz = 4-(benzyl)-1,2,4-triazole; tcnset = 1,1,3,3-tetracyano-2-thioethylepropenide; L = 2,6-di(pyrazol-1-yl)pyridine.
^{b}Transition temperatures measured during heating are tabulated here.
^{c}Entropy of transition ΔSₜᵣ measured at ambient pressure are tabulated here.
^{d}ΔT_{hys} refers to the difference between T_{tr, heating} and T_{tr, cooling} at ambient pressure.
^{e}Pᵣₑᵥ is calculated through Pᵣₑᵥ = ΔT_{hys}/|dTₜᵣ/dP|, and dTₜᵣ/dP values for exothermic and endothermic transitions are used for conventional and inverse barocaloric materials, respectively. Note that inverse barocaloric materials refer to the compounds with dTₜᵣ/dP < 0.
^{f}The reversible isothermal entropy changes, ΔS_{it, rev}, at the driving pressure ΔP are tabulated here. Note that these values were derived from quasi-direct measurements. At high pressure (typically above 1 kbar), the additional entropy change outside of the transition, ΔS₊, plays a role and contributes to ΔSᵢₜ values, leading to ΔSᵢₜ to higher than ΔSₜᵣ. The ΔSᵢₜ measured at higher pressures are shown in the parentheses.
^{g}dTₜᵣ/dP values were averaged from heating and cooling data.
^{h}Only irreversible ΔSᵢₜ value is reported (Ni_{0.85}Fe_{0.15}S, 53 J kg⁻¹ K⁻¹ at 1000 bar; [FeL₂](BF₄)₂, 68 J kg⁻¹ K⁻¹ at 430 bar).
ⁱan irreversible phase transitions occur at the pressure above 4900 bar.
^{j}dTₜᵣ/dP values obtained at a pressure range < 2 kbar values are shown. Note that (dTₜᵣ/dP)_{heating} and (dTₜᵣ/dP)_{cooling} were obtained from calorimetry and SQUID magnetometry, respectively.

TABLE 19
Barocaloric effects and predicted thermodynamic
efficiencies for selected barocaloric materials including
two of the invention ((DA)₂MnCl₄ and (NA)₂CuBr₄).

| | | ΔS_{it, rev}/ΔP^{c} | | ΔT_{hys}/ | |
|---|---|---|---|---|---|
| Chemical | | (J kg⁻¹ K⁻¹ | ΔT_{ad, max}^{d} | ΔT_{ad, max}^{e} | η^{f} |
| Formula^{a} | Tₜᵣ^{b} | kbar⁻¹) | (K) | (%) | (%) |
| (DA)₂MnCl₄ | 310 | 500 | 42^{g} | 3.3 | 89 |
| (NA)₂CuBr₄ | 303 | 453 | 21^{h} | 1.9 | 93 |
| [TPrA][Mn(dca)₃] | 330 | 436 | 5ⁱ | 15.8 | 61 |
| (CH₃)₂C(CH₂OH)₂ | 314 | 178 | 45^{j} | 31.1 | 45 |
| C₆₀ | 259 | 32 | 20^{k} | 15.0 | 63 |
| (NH₄)₂SO₄ | 222 | 60 | 8^{l} | 12.5 | 67 |
| Ni_{0.85}Fe_{0.15}S^{h} | 303 | | 30^{m} | 38.3 | 40 |
| Fe₃(bntrz)₆(tcnset)₆ | 318 | 145 | 35ⁿ | 5.7 | 81 |

^{a}DA = decylammonium; NA = nonylammonium; TPrA = tetrapropylammonium; bntrz = 4-(benzyl)-1,2,4-triazole; tcnset = 1,1,3,3-tetracyano-2-thioethylepropenide.
^{b}Transition temperatures measured during heating are tabulated here.
^{c}The reversible isothermal entropy change ΔS_{it, rev} normalized by the driving pressure, often referred to as barocaloric strength, are tabulated here. Note that the barocaloric strength values were maximized by choosing the smallest ΔP values that can capture the full entropy of the transition.
^{d}Maximum adiabatic temperature changes tabulated here were predicted by indirect methods, with ΔT_{ad, max} = TΔSᵢₜ/cₚ, or, by quasi-direct methods.
^{e}ΔT_{hys} values measured at ambient pressure were used.
^{f}The second-law efficiency η, which corresponds to coefficient of performance (COP) of material with hysteresis normalized by COP of Carnot cycle, is estimated using the equation
$η = COP / {COP}_{Carnot} = \frac{1}{1 + 4 \frac{Δ {T}_{hys}}{Δ {T}_{ad , max}}} .$ Note that this relation is derived from a phenomenological model that integrates the dissipative losses due to hysteresis in a Carnot-like cycle and provides insights into how thermal hysteresis of a material reduces the efficiency.
^{g}Estimated through the indirect method, with ΔS_{it, rev} of 210 J kg⁻¹ K⁻¹ predicted to occur at the driving pressure of 270 bar with T = 312 K and cₚ = 1550 J kg⁻¹ K⁻¹.
^{h}Estimated through the indirect method, with ΔS_{it, rev} of 68 J kg⁻¹ K⁻¹ from the pressure change of 150 bar with T = 306 K and cₚ = 800 J kg⁻¹ K⁻¹.
ⁱEstimated through the indirect method, with ΔS_{it, rev} of 68 J kg⁻¹ K⁻¹ from the pressure change of 150 bar with T = 332 K and cₚ = 2450 J kg⁻¹ K⁻¹.
^{j}Estimated to be around 50 K and later confirmed through quasi-direct measurement to be 45 K at the driving pressure of 5700 bar.
^{k}quasi-direct measurements at the driving pressure of 5.9 kbar.
^{l}Estimated through the indirect method, with ΔS_{it, rev} of 60 J kg⁻¹ K⁻¹ predicted to occur at the driving pressure of 1000 bar with cₚ = 1700 J kg⁻¹ K⁻¹.
^{m}Estimated through the indirect method, with the irreversible ΔSᵢₜ value of 53 J kg⁻¹ K⁻¹ at the driving pressure of 1000 bar.
^{m}Quasi-direct measurements at the driving pressure of 2600 bar.

TABLE 20
Phase-change properties and barocaloric effects
of compounds with long-chain hydrocarbons.

| | | | ΔSₜᵣ^{c} | d Tₜᵣ/d P^{d} | Δ T_{hys}^{d} |
|---|---|---|---|---|---|
| Type | Chemical Formula^{a} | Tₜᵣ^{b} | (J kg⁻¹ K⁻¹) | (K kbar⁻¹) | (K) |
| 2-D perovskite | (OA)₂MnCl₄^{e} | 274 | 153 | 13 | 3.0 |
| | | major | | | |
| | (OA)₂MnCl₄^{e} | 303 | 33 | 40 | 0.7 |
| | | minor | | | |
| | (NA)₂MnCl₄^{e} | 291 | 183 | 20 | 2.2 |
| | | major | | | |
| Di-n-alkyl ammonium | (n-C₆H₁₃)₂NH₂Br | 292 | 291 | 34^{e} | 4.7 |
| salt^{f} | (n-C₈H₁₇)₂NH₂Cl | 294 | 343 | | |
| | (n-C₈H₁₇)₂NH₂Br | 302 | 247 | | |
| | (n-C₁₂H₂₅)₂NH₂Cl | 338 | 379 | | |
| | (n-C₁₂H₂₅)₂NH₂Br | 345 | 327 | | |
| | (n-C₁₈H₃₇)₂NH₂Cl | 366 | 411 | | |
| | (n-C₁₈H₃₇)₂NH₂Br | 370 | 379 | | |
| Intercalation compound | FeOCl•C₁₄H₂₉NH₂^{g} | 323 | 93 | | |
| (first-row transition | Ni(CN)₂•C₁₂H₂₅NH₂^{h} | 327 | 214 | | |
| metal) | | major | | | |
| | Ni(CN)₂•C₁₂H₂₅NH₂^{h} | 363 | 41 | | |
| | | minor | | | |
| intercalated between | (C₁₈H₃₇)₃NH⁺ⁱ | 324 | 83 | | |
| montmorillonite | Self-Assembled | | | | |
| (smectite) | Monolayer | | | | |
| | (C₁₈H₃₇)₄N⁺ⁱ | 310 | 63 | | |
| | Self-Assembled | | | | |
| | Monolayer | | | | |
| Layered metallo- | Mg(O₃PC₂₂H₄₅)^{j} | 290- | 682 | | |
| alkylphosphonate | | 330 | | | |

^{a}DA = decylammonium; NA = nonylammonium.
^{b}Transition temperatures measured during heating are tabulated here.
^{c}Entropy of transition ΔSₜᵣ measured at ambient pressure are tabulated here. The literature values are associated with large uncertainty.
^{d}Barocaloric coefficients tabulated here were measured through high-pressure differential calorimetry under Helium gas environment. Thermal hysteresis were measured at ambient pressure.
^{e}Phase-change properties, including the change in volume, was previously reported.
^{f}Compounds listed here are predicted to display large barocaloric effects, due to high ΔSₜᵣ and large volume change (ΔVₜᵣ)of ~7%.
^{g}Predicted to display large inverse barocaloric effects due to ΔVₜᵣ, = −7%.
^{h}Predicted to display large barocaloric effects due to large ΔVₜᵣ ~11%. However, the reversibility of the major transition requires further investigation.
ⁱPredicted to display large barocaloric effects due to large ΔVₜᵣ of 2% and 5% for (C₁₈H₃₇)₃NH⁺ and (C₁₈H₃₇)₄N⁺, respectively.
^{j}Four successive transitions occur at a temperature range between 290 K and 330 K, with a major transition around 305 K. The total entropy change is tabulated here. To probe reversibility and volume change, further investigations are required.

TABLE 21
Summary of both gravimetric and molar thermodynamic properties
of the phase transition in mixed-halide 2-D perovskites.
The transition temperatures listed are heating values
for the principal transition, and the molar values were
calculated based on the intended halide ratios.

| | | | | ΔS | ΔS |
|---|---|---|---|---|---|
| Chemical | Tₜᵣ | ΔH | ΔH | (J mol⁻¹ | (J kg⁻¹ |
| Formula | (K) | (kJ mol⁻¹) | (kJ kg⁻¹) | K⁻¹) | K⁻¹) |
| (NA)₂CuCl₄ | 295 | 26.8 | 51.7 | 86.1 | 174.3 |
| (NA)₂CuCl₃Br | 290 | 24 | 44.6 | 82.3 | 153.0 |
| (NA)₂CuCl₂Br₂ | 286 | 17.9 | 30.8 | 62.5 | 107.2 |
| (NA)₂CuClBr₃ | 292 | 16.4 | 26.1 | 56.1 | 89.5 |
| (NA)₂CuBr₄ | 302 | 15 | 22.3 | 49.6 | 73.9 |
| (DA)₂CuCl₄ | 309 | 34.5 | 66.2 | 111.4 | 213.5 |
| (DA)₂CuCl₃Br | 308 | 25.6 | 45.3 | 83.3 | 147.1 |
| (DA)₂CuCl₂Br₂ | 307 | 25.2 | 41.2 | 82.0 | 134.2 |
| (DA)₂CuClBr₃ | 309 | 19.8 | 30.2 | 64.2 | 97.9 |
| (DA)₂CuBr₄ | 317 | 18.1 | 25.9 | 57.3 | 81.9 |

TABLE 22
Summary of both gravimetric and molar thermodynamic properties
of the phase transition in mixed-cation 2-D perovskites.

| | | ΔH | ΔH | ΔS | ΔS |
|---|---|---|---|---|---|
| | Tₜᵣ | (kJ | (kJ | (J mol⁻¹ | (J kg⁻¹ |
| Chemical Formula | (K) | mol⁻¹) | kg⁻¹) | K⁻¹) | K⁻¹) |
| (NA)₂CuCl₄ | 295 | 26.8 | 54.3 | 86.1 | 174.3 |
| [(NA)_{0.75}(DA)_{0.25}]₂CuCl₄ | 290 | 19.0 | 44.6 | 65.5 | 130.7 |
| [(NA)_{0.5}(DA)_{0.5}]₂CuCl₄ | 291 | 22.0 | 43.2 | 75.6 | 148.9 |
| [(NA)_{0.25}(DA)_{0.75}]₂CuCl₄ | 297 | 21.7 | 26.1 | 72.9 | 141.6 |
| (DA)₂CuCl₄ | 309 | 34.5 | 22.3 | 111.4 | 213.5 |
| [(NA)_{0.5}(UA)_{0.5}]₂CuCl₄ | 282 | 20.6 | 39.4 | 72.8 | 139.4 |

TABLE 23
Summary of thermodynamic properties of the phase
transition in compositionally engineered 2-D perovskites
with promising barocaloric potentials.

| | | ΔH | ΔH | ΔS | ΔS |
|---|---|---|---|---|---|
| | Tₜᵣ | (kJ | (kJ | (J mol⁻¹ | (J kg⁻¹ |
| Chemical Formula | (K) | mol⁻¹) | kg⁻¹) | K⁻¹) | K⁻¹) |
| (DA)₂CuCl₃Br | 308 | 25.6 | 45.3 | 83.3 | 147.1 |
| (DA)₂CuCl₂Br₂ | 307 | 25.2 | 41.2 | 82.0 | 134.2 |
| (NA_{0.5}DA_{0.5})CuCl₄ | 291 | 22.0 | 43.2 | 75.6 | 148.9 |
| (NA_{0.5}UA_{0.5})₂CuCl₄ | 282 | 20.6 | 39.4 | 72.8 | 139.4 |
| (NA_{0.5}DA_{0.5})₂CuCl₂Br₂ | 285 | 18.2 | 31.45 | 64.0 | 111.5 |

TABLE 24
Summary of thermodynamic trends for the five compounds identified
as promising barocaloric materials. ΔT_{hys} is defined here
as the difference between the peak transition temperature upon
heating and the peak transition temperature upon cooling.

| | Tₜᵣ | ΔH | ΔS | dT/dP | P_{rev, ad} | ΔT_{hys} |
|---|---|---|---|---|---|---|
| Chemical Formula | (K) | (kJ kg⁻¹) | (J kg⁻¹ K⁻¹) | (K kbar⁻¹) | (bar) | (K) |
| (DA)₂CuCl₃Br | 308 | 45.3 | 147.1 | 24.3 | 151 | 2.3 |
| (DA)₂CuCl₂Br₂ | 307 | 41.2 | 134.2 | 25.8 | 129 | 2.3 |
| (NA_{0.5}DA_{0.5})₂CuCl₄ | 291 | 43.2 | 148.9 | 22.4 | 166 | 1.6 |
| (NA_{0.5}UA_{0.5})₂CuCl₄ | 282 | 39.4 | 139.4 | 25.1 | 176 | 2.2 |
| (NA_{0.5}DA_{0.5})₂CuCl₂Br₂ | 285 | 31.45 | 111.5 | 24.1 | 161 | 2.6 |

TABLE 25
Summary of barocaloric properties for mixed halide and mixed cation perovskites. Here, ΔP is defined
as the operating pressure required to achieve the maximum adiabatic temperature change, and is calculated as Δ
T_{ad, max}/(dT/d P). Note that cₚ for the low temperature phase is used for the calculations.

| | Tₜᵣ | ΔS | cₚ | dT/dP | P_{rev, ad} | ΔT_{bys} | ΔT_{ad, max} | ΔP |
|---|---|---|---|---|---|---|---|---|
| Chemical Formula | (K) | (J kg⁻¹ K⁻¹) | (J g⁻¹ K⁻¹) | (K kbar⁻¹) | (bar) | (K) | (K) | (bar) |
| (DA)₂CuCl₃Br | 308 | 147 | 1.50 | 24.3 | 151 | 2.3 | 27 | 1103 |
| (DA)₂CuCl₂Br₂ | 307 | 134 | 1.41 | 25.8 | 129 | 2.3 | 26 | 989 |
| (NA_{0.5}DA_{0.5})₂CuCl₄ | 291 | 149 | 1.50 | 22.4 | 166 | 1.6 | 26 | 1163 |
| (NA_{0.5}UA_{0.5})₂CuCl₄ | 282 | 139 | 1.51 | 25.1 | 176 | 2.2 | 23 | 915 |
| (NA_{0.5}DA_{0.5})₂CuCl₂Br₂ | 285 | 112 | 1.34 | 24.1 | 161 | 2.6 | 21 | 866 |

TABLE 26
Phase-change properties of newly synthesized symmetric dialkylammonium salts.
Preliminary characterizations through high-pressure differential scanning calorimetry
(HP-DSC) in our lab or the Clausius-Clapeyron equation (dTₜᵣ/dP = ΔVₜᵣ/ΔSₜᵣ) indicate
that these candidate compounds are all expected to display high pressure sensitivity
(dTₜᵣ/dP) between 10-30K kbar⁻¹. Note that the temperature of transition measured
during the first heating scans are tabulated here.

| | | | ΔSₜᵣ | | ΔSₜᵣ | |
|---|---|---|---|---|---|---|
| | Tₜᵣ | | (J kg⁻¹ | | (J mol⁻¹ | dTₜᵣ/dP |
| | (° C.) | | K⁻¹) | ΔSₜᵣ | K⁻¹) | (K kbar⁻¹) |
| | major | ΔT_{hys} | major | (J L⁻¹ | per chain | major |
| Candidates | (minor) | (° C.) | (minor) | K⁻¹) | (total) | (minor) |
| (C₁₂H₂₅)(CH₃)NH₂Br | 69.6 | 1.4 | 300 | 351 | 84 | 23.1 |
| | | | | | | 22.2 |
| (C₁₂H₂₅)(CH₃)NH₂Cl | 61.1 (58.6); | 2.5 | 350 | 363 | 83 | 23.1 (36) |
| | 58.6 (48.1) | (10.5) | (14) | (15) | | 24.5 (42.7) |

| | | | ΔS | |
|---|---|---|---|---|
| | Tₜᵣ | ΔH | (J K⁻¹ | dT/dP |
| Chemical Formula | (K) | (kJ kg⁻¹) | kg⁻¹) | (K kbar⁻¹) |
| (C₆H₁₃)₂NH₂Cl | 277 | 59.5 | 215 | — |
| (C₈H₁₇)₂NH₂Cl | 292 | 105.5 | 361 | |
| (C₁₀H₂₁)₂NH₂Cl | 321 | 125.0 | 390 | |
| (C₆H₁₃)₂NH₂Br | 295 | 88.3 | 308 | 22-27 |
| (C₈H₁₇)₂NH₂Br | 301 | 80.1 | 266 | |
| (C₁₀H₂₁)₂NH₂Br | 377 | 103.7 | 317 | |
| (C₆H₃₃)₂NH₂I | 284 | 56.6 | 199 | 16-25 |
| (C₈H₁₇)₂NH₂I | 285 | 47.7 | 167 | |
| (C₁₀H₂₁)₂NH₂I | 317 | 81.0 | 256 | |

Table 27. Phase-change properties of newly synthesized asymmetric dialkylammonium salts. Preliminary characterizations through high-pressure differential scanning calorimetry (HP-DSC) indicate that these candidate compounds all display high pressure sensitivity (dTₜᵣ/dP) between 20-30 K kbar⁻¹.

TABLE 28
Unit cell parameters for (CₙH₂ₙ₊₁)₂NH₂Br (n = 6, 8, 10)
from single-crystal X-ray diffraction at ambient pressure.

| | | | | | | | | | Space |
|---|---|---|---|---|---|---|---|---|---|
| Compound | T (K) | a (Å) | b (Å) | c (Å) | α (°) | β (°) | γ (°) | V (Å³) | Group |
| (C₆H₁₃)₂NH₂Br | 100 | 26.267(2) | 5.3453(3) | 10.7542(6) | 90 | 98.299(1) | 90 | 1494.14 | C2/c |
| (C₈H₁₇)₂NH₂Br | 100 | 5.372(2) | 33.568(1) | 5.279(2) | 90 | 90 | 90 | 951.895 | P2₁2₁2 |
| (C₁₀H₂₁)₂NH₂Br | 100 | 5.3475(4) | 39.886(3) | 5.2758(4) | 90 | 90 | 90 | 1125.28 | P2₁2₁2 |

TABLE 29
Unit cell parameters for (C₁₂H₂₅)N(CH₃)H₂X (X = Cl, Br)
from single-crystal X-ray diffraction at ambient pressure at 100K.

| | | | | | | | | Space |
|---|---|---|---|---|---|---|---|---|
| Compound | a (Å) | b (Å) | c (Å) | α (°) | β (°) | γ (°) | V (Å³) | Group |
| (C₁₂H₂₅)(CH₃)NH₂Cl | 4.8858(3) | 5.2510(2) | 29.585(1) | 93.453(4) | 94.633(4) | 90.810(4) | 755.012 | P1̅ |
| (C₁₂H₂₅)(CH₃)NH₂Br | 5.3299(4) | 5.3390(4) | 28.010(2) | 90 | 90.581(3) | 90 | 797.021 | P2₁ |

Other embodiments are in the claims.

What is claimed is:

1. A method of heating or cooling employing a barocaloric cycle comprising:
a) providing heat energy to a composition comprising an organic layer comprising optionally substituted C_{>3} alkyl chains, wherein the organic layer is in a disordered state and wherein the organic layer is between first and second inorganic layers or comprises a head group capable of hydrogen bonding, halogen bonding, and/or electrostatic interaction with a counterion;
b) applying compression to the composition to induce the organic layer to undergo an exothermic phase transition to an ordered state, releasing latent heat;
c) removing the latent heat while the composition is compressed; and
d) removing the compression to allow the composition to revert to the disordered state.

2. The method of claim 1, wherein the composition comprises first and second inorganic layers separated by the organic layer.

3. The method of claim 1, wherein the compression is hydrostatic or mechanical and/or the latent heat is removed by a heat sink.

4. The method of claim 1 or claim 2, wherein the organic layer comprises a C_{>3} alkyl ammonium species.

5. The method of claim 4, wherein the C_{>3} alkyl ammonium species is selected from:

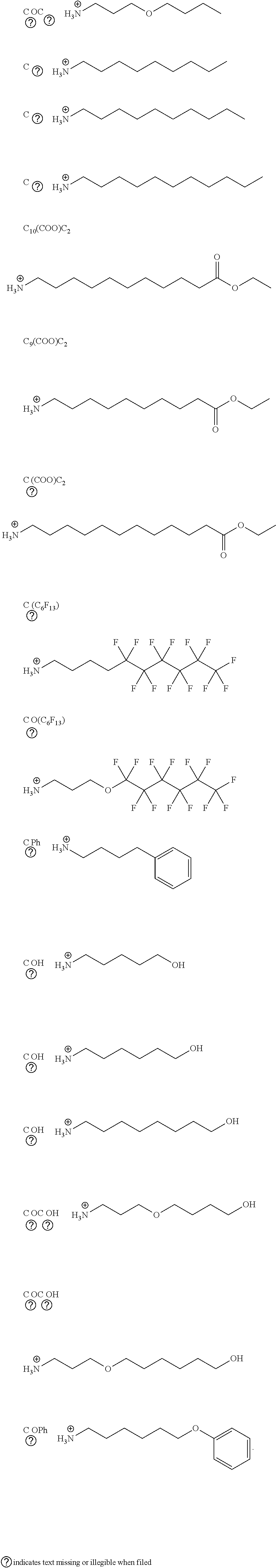

6. The method of any one of claims 1-5, wherein the organic layer is an organic bilayer.

7. The method of any one of claims 1-6, wherein the organic layer comprises a compound of formula (CₙH₂ₙ₊₁)(CₘH₂ₘ₊₁)NH₂X, wherein n is 1-3 or 4-36 and m is 4-36; and wherein X is a monoanionic species.

8. The method of claim 6, wherein the monoanionic species is a halide and/or wherein n=m and n=4-36 or wherein n=1-3 and m=4-36.

9. The method of any one of claims 1-6, wherein the composition is a 2D perovskite.

10. The method of claim 7, wherein the 2D perovskite comprises a transition metal halide.

11. The method of claim 10, wherein the 2D perovskite comprises Ti, V, Cr, Mn, Fe, Co, Ni, Cu, Zn, Nb, Mo, Rh, Pd, Cd, Re, Pt, or Hg.

12. The method of claim 11, wherein the 2D perovskite comprises a tetrahedral or octahedral transition metal complex.

13. The method of claim 12, wherein the halide of the transition metal halide is F, Cl, Br, or I.

14. The method of claim 13, wherein the transition metal halide comprises a monovalent metal cation and a trivalent metal cation.

15. The method of any one of claims 9-14, wherein the 2D perovskite is of formula [(R¹)ₓ(R²)₁₋ₓ]₂MX_{y}X′_{4-y}, wherein R¹ and R² are independently optionally substituted alkylammonium species, wherein X and X′ are different halides, wherein x is between 0-1, wherein y is 0-4, wherein M is a transition metal; and wherein if y=0 or 4, R¹≠R² and x≠0 or 1.

16. The method of any one of claims 1-15, wherein the organic layer comprises two different molecular structures.

17. The method of any one of claims 1-6, wherein the first and second inorganic layers comprise a silicate.

18. The method of any one of claims 1-6, wherein the composition comprises a metal alkyl phosphonate salt.

19. The method of any one of claims 1-6, wherein the composition comprises a compound of the following table:

| Type | Chemical Formula |
|---|---|
| 2-D perovskite | (OA)₂MnCl₄ |
| | (OA)₂MnCl₄ |
| | (NA)₂MnCl₄ |
| | (NA)₂CuCl₄ |
| | (DA)₂CuCl₄ |
| Mixed 2D perovskites | (NA)₂CuCl₃Br |
| | (NA)₂CuCl₂Br₂ |
| | (NA)₂CuClBr₃ |
| | (DA)₂CuCl₃Br |
| | (DA)₂CuCl₂Br₂ |
| | (DA)₂CuClBr₃ |
| | [(NA)_{0.75}(DA)_{0.25}]₂CuCl₄ |
| | [(NA)_{0.5}(DA)_{0.5}]₂CuCl₄ |
| | [(NA)_{0.25}(DA)_{0.75}]₂CuCl₄ |
| | [(NA)_{0.25}(UA)_{0.75}]₂CuCl₄ |
| | [(NA)_{0.5}(UA)_{0.5}]₂CuCl₄ |
| | [(NA)_{0.5}(DA)_{0.5}]₂CuCl₂Br₂ |
| Di-n-alkyl ammonium salt | (n-C₆H₁₃)₂NH₂Br |
| | (n-C₈H₁₇)₂NH₂Cl |
| | (n-C₆H₁₃)₂NH₂Cl |
| | (n-C₆H₁₃)₂NH₂I |
| | (n-C₈H₁₇)₂NH₂Br |
| | (n-C₁₂H₂₅)₂NH₂Cl |
| | (n-C₈H₁₇)₂NH₂I |
| | (n-C₁₀H₂₁)₂NH₂Cl |
| | (n-C₁₀H₂₁)₂NH₂Br |
| | (n-C₁₀H₂₁)₂NH₂I |
| | (n-C₁₂H₂₅)₂NH₂Br |
| | (n-C₁₈H₃₇)₂NH₂Cl |
| | (n-C₁₈H₃₇)₂NH₂Br |
| | (n-C₁₂H₂₅)(CH₃)NH₂Br |
| | (n-C₁₂H₂₅)(CH₃)NH₂Cl |
| Intercalation compound | FeOCl•C₁₄H₂₉NH₂^{g} |
| (first-row transition metal) | Ni(CN)₂•C₁₂H₂₅NH₂^{h} |
| | Ni(CN)₂•C₁₂H₂₅NH₂^{h} |
| intercalated between | (C₁₈H₃₇)₃NH⁺ |
| montmorillonite (smectite) | Self-Assembled Monolayer |
| | (C₁₈H₃₇)₄N⁺ |
| | Self-Assembled Monolayer |
| Layered metallo- | Mg(O₃PC₂₂H₄₅) |
| alkylphosphonate | |

20. The method of any one of claims 1-19, wherein the compression results from a pressure change of less than 500 bar.

21. The method of claim 20, wherein the compression results in a reversible entropy change of more than 200 J kg⁻¹ K⁻¹.

22. The method claim 1, wherein the compression is provided using a pressure transmitting medium (PTM).

23. The method of claim 22, further comprising providing a gas to the PTM that induces a change in a thermal property of the composition.

24. The method of claim 23, wherein the change in thermal property is a lowering of a phase transition temperature and/or a barocaloric effect inversion.

25. The method of claim 24, further comprising removing the gas from the PTM.

26. The method of any one of claims 23-25, wherein the gas is an inert gas that permeates into a free volume of the organic layer.

27. The method of claim 26, wherein the permeated gas interacts with the composition.

28. The method of claim 27, wherein permeation and interaction of the gas with the composition together induce a lowering of a phase transition and/or a barocaloric effect inversion.

29. The method of any one of claims 23-28, wherein the gas is nitrogen, argon, krypton, xenon, methane, ethane, propane, butane, sulfur hexafluoride, or carbon dioxide.

30. A method of storing thermal energy comprising:
a) providing a composition comprising an organic layer comprising optionally substituted C_{>3} alkyl chains at a first temperature and a first pressure, wherein the composition is in an ordered state and wherein the organic layer is between first and second inorganic layers or comprises a head group capable of hydrogen bonding, halogen bonding, and/or electrostatic interaction with a counterion; and
b) reducing compression on the composition to a second pressure to induce a phase transition in the composition to a disordered state, thereby storing energy.

31. The method of claim 30, further comprising increasing compression on the composition to apply a third pressure to revert the composition to an ordered state and release heat energy.

32. The method of claim 30 or 31, wherein the composition comprises first and second inorganic layers separated by the organic layer.

33. The method of any one of claims 30-32, wherein the compression is hydrostatic or mechanical.

34. The method of any one of claims 30-33, wherein the organic layer comprises a C_{>3} alkyl ammonium species.

35. The method of claim 34, wherein the C_{>3} alkyl ammonium species is selected from:

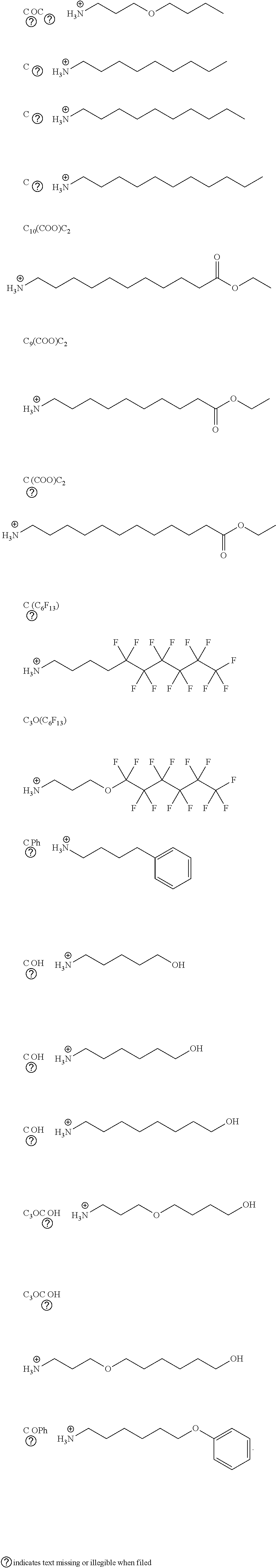

36. The method of any one of claims 30-35, wherein the organic layer is an organic bilayer.

37. The method of any one of claims 30-36, wherein the organic layer comprises a compound of formula (CₙH₂ₙ₊₁)(CₘH₂ₘ₊₁)NH₂X, wherein n is 1-3 or 4-36 and m is 4-36; and wherein X is a monoanionic species.

38. The method of claim 37, wherein the monoanionic species is a halide and/or wherein n=m and n=4-36 or wherein n=1-3 and m=4-36.

39. The method of any one of claims 30-36, wherein the composition is a 2D perovskite.

40. The method of claim 39, wherein the 2D perovskite comprises a transition metal halide.

41. The method of claim 39, wherein the 2D perovskite comprises Ti, V, Cr, Mn, Fe, Co, Ni, Cu, Zn, Nb, Mo, Rh, Pd, Cd, Re, Pt, or Hg.

42. The method of claim 39, wherein the 2D perovskite comprises a tetrahedral or octahedral transition metal complex.

43. The method of claim 40, wherein the halide of the transition metal halide is F, Cl, Br, or I.

44. The method of claim 40, wherein the transition metal halide comprises a monovalent metal cation and a trivalent metal cation.

45. The method of any one of claims 40-44, wherein the 2D perovskite is of formula [(R¹)ₓ(R²)₁₋ₓ]₂MX_{y}X′_{4-y}, wherein R¹ and R² are independently optionally substituted alkylammonium species, wherein X and X′ are different halides, wherein x is between 0-1, wherein y is 0-4, wherein M is a transition metal; and wherein if y=0 or 4, R¹≠R² and x≠0 or 1.

46. The method of any one of claims 30-45, wherein the organic layer comprises two different molecular structures.

47. The method of any one of claims 30-36, wherein the inorganic layer comprises a silicate.

48. The method of any one of claims 30-36, wherein the composition comprises a metal alkyl phosphonate salt.

49. The method of any one of claims 30-36, wherein the composition comprises a compound of the following table:

| Type | Chemical Formula |
|---|---|
| 2-D perovskite | (OA)₂MnCl₄ |
| | (OA)₂MnCl₄ |
| | (NA)₂MnCl₄ |
| | (NA)₂CuCl₄ |
| | (DA)₂CuCl₄ |
| Mixed 2D perovskites | (NA)₂CuCl₃Br |
| | (NA)₂CuCl₂Br₂ |
| | (NA)₂CuClBr₃ |
| | (DA)₂CuCl₃Br |
| | (DA)₂CuCl₂Br₂ |
| | (DA)₂CuClBr₃ |
| | [(NA)_{0.75}(DA)_{0.25}]₂CuCl₄ |
| | [(NA)_{0.5}(DA)_{0.5}]₂CuCl₄ |
| | [(NA)_{0.25}(DA)_{0.75}]₂CuCl₄ |
| | [(NA)_{0.25}(UA)_{0.75}]₂CuCl₄ |
| | [(NA)_{0.5}(UA)_{0.5}]₂CuCl₄ |
| | [(NA)_{0.5}(DA)_{0.5}]₂CuCl₂Br₂ |
| Di-n-alkyl ammonium | (n-C₆H₁₃)₂NH₂Br |
| salt | (n-C₆H₁₃)₂NH₂Cl |
| | (n-C₆H₁₃)₂NH₂I |
| | (n-C₈H₁₇)₂NH₂Cl |
| | (n-C₈H₁₇)₂NH₂Br |
| | (n-C₈H₁₇)₂NH₂I |
| | (n-C₁₀H₂₁)₂NH₂Cl |
| | (n-C₁₀H₂₁)₂NH₂Br |
| | (n-C₁₀H₂₁)₂NH₂I |
| | (n-C₁₂H₂₅)₂NH₂Cl |
| | (n-C₁₂H₂₅)₂NH₂Br |
| | (n-C₁₈H₃₇)₂NH₂Cl |
| | (n-C₁₈H₃₇)₂NH₂Br |
| | (n-C₁₂H₂₅)(CH₃)NH₂Br |
| | (n-C₁₂H₂₅)(CH₃)NH₂Cl |
| Intercalation compound | FeOCl•C₁₄H₂₉NH₂ |
| (first-row transition metal) | Ni(CN)₂•C₁₂H₂₅NH₂ |
| | Ni(CN)₂•C₁₂H₂₅NH₂ |
| intercalated between | (C₁₈H₃₇)₃NH⁺ |
| montmorillonite (smectite) | Self-Assembled |
| | Monolayer |
| | (C₁₈H₃₇)₄N⁺ |
| | Self-Assembled |
| | Monolayer |
| Layered metallo- | Mg(O₃PC₂₂H₄₅) |
| alkylphosphonate | |

50. The method claim 30, wherein the compression is provided using a pressure transmitting medium (PTM) and further comprising providing a gas to the PTM that induces a change in a thermal property of the composition.

51. The method of claim 50, wherein the gas is an inert gas that permeates into a free volume of the organic layer.

52. The method of claim 51, wherein the permeated gas interacts with the composition.

53. The method of claim 52, wherein permeation into and interaction of the gas with the composition together induce a lowering of a phase transition and/or a barocaloric effect inversion.

54. The method of any one of claims 50-53, wherein the gas is nitrogen, argon, krypton, xenon, methane, ethane, propane, butane, sulfur hexafluoride, or carbon dioxide.

55. The method of any one of claims 50-54, wherein the change in thermal property is a lowering of a phase transition temperature and/or a barocaloric effect inversion.

56. A 2D perovskite composition comprising:
a) first and second layers of a transition metal halide; and
b) an organic layer comprising a C_{>3} alkyl ammonium species selected from:

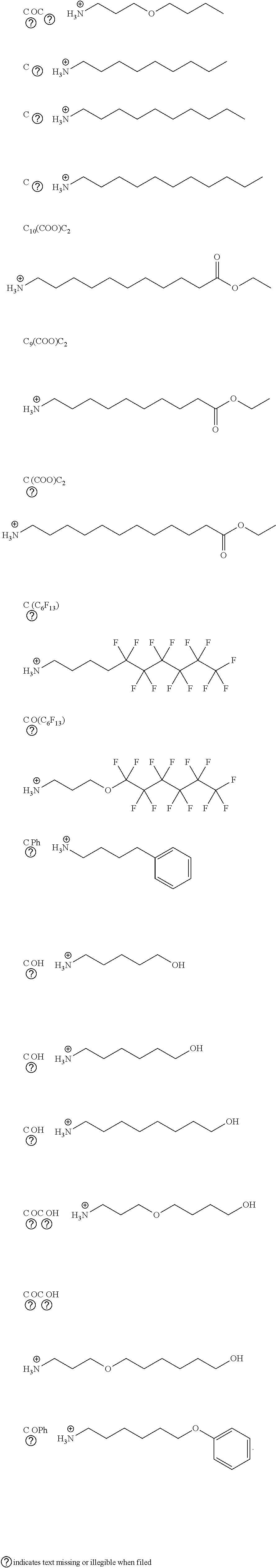

57. A 2D perovskite composition having formula [(R¹)ₓ(R²)₁₋ₓ]₂MX_{y}X′_{4-y}, wherein R¹ and R² are independently optionally substituted alkylammonium species, wherein X and X′ are different halides, wherein x is between 0-1, wherein y is 0-4, wherein M is a transition metal; and wherein if y=0 or 4, R¹≠R² and x≠0 or 1.

58. The 2D perovskite composition of claim 57, wherein R¹ and R² are independently alkylammonium species of formula CₙH₂ₙ₊₁NH₃⁺, wherein n>3.

59. The 2D perovskite composition of claim 57, wherein the composition has a formula selected from:
(NA)₂CuCl₃Br; (NA)₂CuCl₂Br₂; (NA)₂CuClBr₃; (DA)₂CuCl₃Br; (DA)₂CuCl₂Br₂; (DA)₂CuClBr₃;
[(NA)_{0.75}(DA)_{0.25}]₂CuCl₄; [(NA)_{0.5}(DA)_{0.5}]2CuCl₄; [(NA)_{0.25}(DA)_{0.75}]2CuCl₄; [(NA)_{0.25}(UA)_{0.75}]₂CuCl₄;
[(NA)_{0.5}(UA)_{0.5}]₂CuCl₄; or [(NA)_{0.5}(DA)_{0.5}]₂CuCl₂Br₂; wherein DA=decylammonium, NA=nonylammonium, and UA=undecylammonium.

60. The 2D perovskite composition of claim 57, wherein R¹ and R² are independently alkylammonium species selected from:

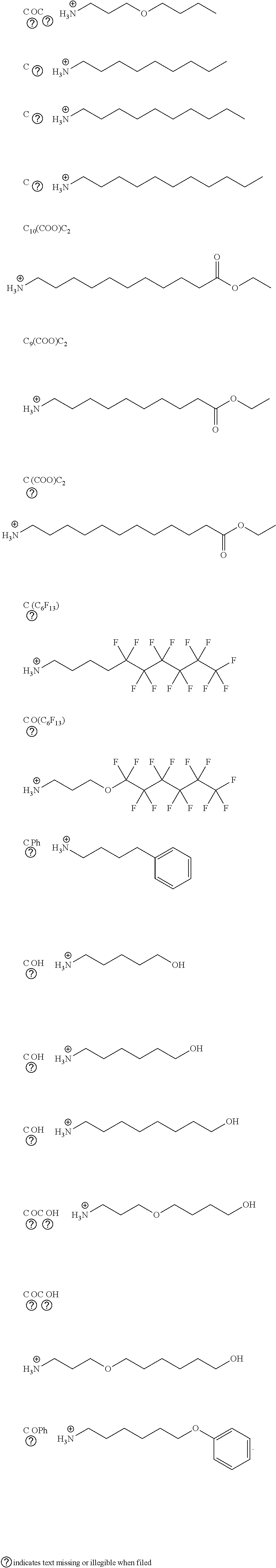

61. The 2D perovskite composition of any one of claims 57-60, wherein X is Cl and X′ is Br.

62. A barocaloric system comprising:
a) a composition comprising an organic layer comprising optionally substituted C_{>3} alkyl chains, wherein the organic layer is between first and second inorganic layers or comprises a head group capable of hydrogen bonding, halogen bonding, and/or electrostatic interaction with a counterion; and
b) a source of compression.

63. The system of claim 62, further comprising first and second inorganic layers separated by the organic bilayer.

64. The system of claim 62, wherein the organic layer includes a compound of formula (CₙH₂ₙ₊₁)(CₘH₂ₘ₊₁)NH₂X, wherein n is 1-3 or 4-36 and m=4-36; and wherein X is a monoanionic species.

65. The system of claim 64, wherein the monoanionic species is a halide and/or wherein n=m and n=4-36 or wherein n=1-3 and m=4-36.

66. The system of any one of claims 62-65, wherein the source of compression is hydrostatic or mechanical.

67. The system of any one of claims 62-66, further comprising a heat sink.

68. A barocaloric system comprising:
a) a composition comprising an organic layer comprising optionally substituted C_{>3} alkyl chains;
b) a pressure transmitting medium comprising one or more gases, wherein at least one gas of the one or more gases induces a change in a thermal property of the organic layer; and
c) a source of compression.

69. The system of claim 68, wherein the at least one gas is an inert gas that is able to permeate a free volume of the organic layer.

70. The system of claim 69, wherein the permeated gas interacts with the composition.

71. The system of claim 70, wherein an extent of permeation and interaction of the at least one gas with the composition together induce a lowering of a phase transition and/or a barocaloric effect inversion.

72. The system of any one of claims 68-71, wherein the change in thermal property is a lowering of a phase transition temperature and/or a barocaloric effect inversion.

73. The system of any one of claims 68-72, further comprising a pump for controlling the amount of the at least one gas.

74. The system of any one of claims 68-73, further comprising a heat sink.

75. The system of claim 72, wherein the change in thermal property comprises the barocaloric effect inversion, further comprising a second organic layer that does not undergo the barocaloric effect inversion.

76. The system of any one of claims 68-75, wherein the at least one gas is nitrogen, argon, krypton, xenon, methane, ethane, propane, butane, sulfur hexafluoride, or carbon dioxide.
